(19) United States
(12) Patent Application Publication
(10) Pub. No.: US 2025/0136942 A1
(43) Pub. Date: May 1, 2025

(54) METHODS TO ACCELERATE STEM CELL DIFFERENTIATION TO CARDIOMYOCYTES

(71) Applicant: UNIVERSITY OF SOUTHERN CALIFORNIA, Los Angeles, CA (US)

(72) Inventors: Zhipeng LU, Los Angeles, CA (US); Minjie ZHANG, Los Angeles, CA (US); Kongpan LI, Los Angeles, CA (US); Jianhui BAI, Los Angeles, CA (US)

(21) Appl. No.: 18/932,104

(22) Filed: Oct. 30, 2024

Related U.S. Application Data
(60) Provisional application No. 63/594,213, filed on Oct. 30, 2023.

(51) Int. Cl. C12N 5/077; A61K 35/34; C12N 9/22; C12N 15/113

(52) U.S. Cl. CPC C12N 5/0657; A61K 35/34; C12N 9/22; C12N 15/113; C12N 2310/11; C12N 2310/141; C12N 2310/20; C12N 2506/02; C12N 2506/45

(57) ABSTRACT

The disclosure provides methods of promoting stem cell differentiation into mesoderm and endoderm cell lineages comprising, for example, contacting a stem cell with an agent that reduces or eliminates gene activity of one or more endogenous small nuclear ribonucleic acid (snoRNA) molecules, wherein the snoRNA molecules comprise SNORD97 and/or SNORD133. Also provided are isolated cardiomyocytes and compositions of the same, wherein the cardiomyocytes are produced using a method described herein.

### RELATED APPLICATIONS

This application claims priority under 35 U.S.C. § 119(e) to U.S. Provisional Patent Application No. 63/594,213 filed Oct. 30, 2023, which is incorporated herein by reference.

### STATEMENT REGARDING FEDERAL FUNDING

This invention was made with government support under grant nos. R00HG009662, R01HG012928, and R35GM143068, awarded by the (NIH) National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Oct. 30, 2024, is named 530041US1 and is 101,843 bytes in size.

### BACKGROUND OF THE INVENTION

Codon and amino acid usage is non-random in mRNAs across three domains of life. The tRNA pool is dynamically regulated in development and in response to the environment via tissue-specific expression, chemical modification, splicing and charging (aminoacylation). Biased usage of codons and amino acids in mRNAs, and corresponding changes in the tRNA pool create regulatory mechanisms for translation speed, protein function, and gene expression programs in development. Disrupted balance underlies various human diseases. For example, methionine (Met) is highly enriched in proteins involved in basic cellular processes, such as translation, splicing and mitochondrial respiration. The ability of Met to scavenge reactive oxygen species (ROS) by reversible oxidation and repair is essential in protecting important proteins with long half-lives and/or close to ROS source. Both nuclear and mitochondrial genome encoded mitochondrial proteins are enriched in Met, reflecting a convergent evolution in the usage of codons and amino acids. However, factors that regulate global tRNA supply and mRNA codon demand remain largely unknown.

Small nucleolar (sno)RNAs are a large family of noncoding (nc)RNAs in eukaryotes and archaea that often use antisense guide sequences to recognize RNA targets. The human genome encodes ˜2000 snoRNAs, many of which are differentially expressed in cell types and development. Most snoRNAs are classified into two types, where C/D snoRNAs guide the 2′-O-methyltransferase (MTase) Fibrillarin (FBL) to catalyze 2′-O-methylation (Nm), and H/ACA snoRNAs guide the pseudouridine synthase Dyskerin (DKC1) to catalyze pseudouridylation (Ψ). Evidence for additional snoRNA targets, such as mRNAs, tRNAs, and other ncRNAs, remain limited and sometimes controversial. The vast majority of snoRNAs have no known targets and are called orphans. Genetic studies have linked snoRNAs to many physiological and pathological conditions, such as the neurodevelopmental disorder—Prader Willi Syndrome, metabolic disorders, viral infections, and cancer, however, our limited knowledge of snoRNA targets made it difficult to study their functions.

### SUMMARY OF THE INVENTION

The dynamic equilibrium between tRNA supply and codon usage demand is a fundamental mechanism in gene expression, yet the regulators and consequences remain poorly understood. On the other hand, the targets and functions for the vast majority of the large family of snoRNAs (>2000 in human) remain unknown. In this study, we used multiple approaches to discover a large snoRNA interactome, including nearly all nuclear-encoded tRNAs. These interactions control tRNA modifications, stability, and levels, and affect dichotomous codon-biased gene expression programs in proliferation vs. development in human HEK293 cells, and a mouse embryonic stem cell differentiation model. Together, our work revealed a snoRNA-controlled cellular translation economy: specific snoRNAs regulate target tRNA “supply”, which influences the corresponding mRNA codon usage “demand”.

Accordingly, the disclosure provides for methods of promoting stem cell differentiation into mesoderm and endoderm cell lineages comprising contacting a stem cell with an agent that reduces or eliminates gene activity of one or more endogenous small nuclear ribonucleic acid (snoRNA) molecules, wherein the snoRNA molecules comprise SNORD97 and/or SNORD133.

In other embodiments, the disclosure provides for an isolated cardiac cell differentiated from stem cells, wherein endogenous SNORD97 and/or SNORD133 gene activity of the isolated cardiac cell has been eliminated or reduced compared to a reference cell.

In other embodiments, a composition comprising in vitro differentiated stem cells, wherein the in vitro differentiated stem cells are genetically modified to lack, or have decreased or disrupted expression and/or activity of, one or more small nuclear ribonucleic acid (snoRNA) molecules, wherein the snoRNA comprises SNORD97 and SNORD133.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-C. D97/D133 snoRNAs target an extensive set of tRNAs. (A) D97/D133 and eMet-CAU snoRNA-tRNA sub-networks (B). (C) dRMS analysis of D97/D133 target tRNAs in siCtrl, siFBL and D97/D133 double KO HEK293 cells. RMscore: riboMeth-seq score. P values: unpaired two-sided t-tests. n.s.: not significant. n=4 samples for each group.

FIG. 2A-H. D97/D133 snoRNAs balance the tRNA pool for efficient translation. (A) Proliferation of WT and single and double KO cells (two clones each). P values from two-sided unpaired t-tests. P values for D97 KO vs. WT: 0.30 and 0.38 (day 4), 0.0024 and 0.0084 (day 5). P values for D133 KO vs. WT: 0.0005 and 0.0004 (day 4), 3.2E-05 and 3.6E-06 (day 5). P values for D97/D133 double KO vs. WT: 0.0001 and 5.8E-05 (day 4), 2.4E-06 and 2.5E-06 (day 5). (B) KO cell lines were rescued by snoRNA overexpression (OE). P values <0.01 are indicated in the figure (asterisks *, for all clones), based on two-sided unpaired t-tests. (C-D) Nascent proteins were labeled using HPG, detected using Alexa-Fluor-647 azide (C), quantified and normalized against total protein on the gel (D). (E) All 432 nuclear tRNA genes were grouped by anticodons and then log 2 transformed ratios between KO and WT were normalized so that median=1 for each KO/WT comparison. Error bars: standard deviations. (F) Rescue of cell growth defects by overexpressing eMet-CAU or 7 tRNAs in WT and KO cell lines. (G) Bicistronic luciferase reporter plasmids. 6×Met-ATG oligo was inserted after the start codon of Fluc. (H) WT and KO HEK293 cells were transfected with plasmids in panel G. The y axis of normalized Fluc/Rluc ratio corresponding control transfected control plasmid in the same cell line. All data are representative of at least three independent experiments. p values were shown for each KO cell line relative to WT.

FIG. 3A-D. D97/D133 govern Met-biased gene expression programs. (A) For nuclear-encoded mRNAs in RNA-seq (upper) or ribo-seq (lower) from WT and D97/D133 double KO HEK293 cells, ratios of expression levels were ranked. Usage of 66 codons—64 plus iMet and Sec—were calculated for the top 10% (up-regulated) and bottom 10% (down-regulated) mRNAs and weighted by expression levels. Asterisk (*): stop codons. M and m: eMet and iMet. U_uga: Sec. (B) Same as panel A, except that amino acid usage was calculated. n=23 for 20 amino acids plus iMet, stop, and Sec. (C) Changes in codon and anticodon usage between D97/D133 double KO and WT. RNA-seq codon: usage was calculated for 66 codons in mRNAs from KO and WT. Ribo-seq codon: same as RNA-seq codon, except that ribo-seq was used. RNA-seq tRNA anticodon freq: tRNA levels were measured in the RNA-seq. Left: RNA-seq vs. ribo-seq codon usage. The inset x-axis ratio=1.276 indicates ratio of average NN[GC] vs. average NN[AU] in RNA-seq. The inset y-axis ratio=1.098 indicates the ratio of average NN[GC] vs. average NN[AU] in ribo-seq. p values after the ratios: two-sided unpaired t-tests between the two codon groups. Middle panel: RNA-seq mRNA codon freq. vs. RNA-seq tRNA codon frequency. Codons recognized by the same tRNA anticodons were merged. Right panel: same as the middle panel, except that ribo-seq mRNA codon frequency is the y-axis. (D) Same as panel C, except that the amino acid usage was plotted.

FIG. 4A-D. Transcriptome and translatome codon usage depends on snoRNA dose. (A) Ratios of mRNA levels in KO vs. WT for RNA-seq and ribo-seq. mRNAs plotted: n=1682. Linear regression results on the right. (B) Nuclear tRNA anticodon groups (n=48), mRNA codon (n=66), and mRNA amino acid (n=23) usage changes upon D97/D133 single and double KO. Panels 1-2: abundance of tRNA anticodons in KO vs. WT (WT set to 1). Panels 3-6: mRNA codon and amino acid usage on the transcriptome level. Panels 7-10: mRNA codon and corresponding amino acid usage on the translatome level. (C) Standard deviations (sd) of anticodon, codon and amino acid usage plots in panel B. (D) Model for the effects of snoRNA-guided modifications on the tRNA pool, mRNA codon usage, translation, and cellular states. Single and double KO affect the transcriptome and translatome to different degrees.

FIG. 5A-O. Mouse D97 and D133 regulate stem cell differentiation. (A) Diagram of mES differentiation into embryoid bodies that contain three germ layers. (B-D) Expression of the snoRNAs, markers for pluripotency and germ layers, and cardiomyocyte mRNA Myh6 from EBs were measured by qRT-PCR. P values: unpaired two-sided t-tests. (E) Proliferation of WT and D133 KO cells (clones #4 and #5). (F-H) WT and D133 KO mES cells (3 clones) were differentiated into CMs. Beating patches of CM cells were counted every 3 days. p values are from two-sided t-test, between each KO cell line and WT. For (E), (G), and (H), circle is WT, square is D133-KO #4, triangle is D133-KO #5, and diamond is D133-KO #20. (I) Expression of pluripotency and CM mRNAs in WT and D133 KO mES cells, measured by qRT-PCR. (J-K) Pluripotency factor protein levels in WT and D133 KO mES cells, measured by western blots. (L-G) Expression dynamics of pluripotency factors (L), cardiac chamber morphogenesis factors (M), and mitochondrial RNAs (N-O), for the two genotypes across ES, EB, and CM. Stage specific expression of mitochondrial RNAs are summarized in violin and box plots (N-O).

FIG. 6A-C. Characterization of the D97/D133 sub-network. (A-B) R-scape and CaCofold analysis of the interactions between eMet-CAU/Leu-CAA tRNAs and their guide RNAs across eukaryotes and archaea. Alignments are numbered 1-45 (20 nt tRNA, 5 nt N and 20 nt guide). (B) shows the statistics for all base pairs proposed by R-scape. Boxes and asterisks indicate significant covariation above evolutionary background. (C) Construction of D97/D133 KO HEK293 cells. EIF4G2 hosts only D97. LARP4 hosts only D133. Open arrows represent snoRNAs. Open rectangles represent neighbor exons in host genes. The forward (F) and reverse (R) primers are used for PCR validation. The genomic sequences targeted by sgRNAs are shown in sense orientation. The PAM (or complementary) sequences are in bold italics. PCR of genomic DNA from snoRNA KO HEK293 cells were visualized on 2% agarose gels. The double KO was made from D97 KO. taaaaagacgcgttattaagagg (SEQ ID NO: 26), gggagtatagagtattagaagcg (SEQ ID NO: 27), ataattagaagaatcgaatctgg (SEQ ID NO: 28), gggagtctagagtattagaatgg (SEQ ID NO: 29).

FIG. 7A-D. D97/D133 loss induces tRNA fragmentation. (A) For each experimental condition, reads mapped to each gene from the two replicates of small RNA-seq data were plotted. Pearson's correlation coefficients were calculated. D97 KO r=0.996; D133 KO r=0.9987; D97/D133 KO r=0.9984. (B) Changes of 15-50 nt RNA levels upon snoRNA KO. Y-axis is the ratios of RNAs RPM values (reads per million) in KO vs. WT. Each value was added 50 to avoid the division-by-zero problem and reduce variation. (C)-(D) Met tRFs from HEK293 cells. Individual tRFs were extracted and plotted as rectangles. Each rectangle is one group of identical reads defined by the start and end, where height is the number of reads. Arrow: start the 3′ tRNA half. Bars in the mutation track are modified positions that induce RT stops and mutations.

FIG. 8A-H. Characterization of D97/D133 KO HEK293 cells. (A-B) Pearson correlation for 48 nuclear-encoded tRNA types between HEK293 D97/D133 single and double KO clones in total RNA-seq and Ribo-seq. r is Pearson correlation coefficient. n is the number of genes/RNAs detected at >=10 reads. (C) Confirmation of Met-CAU expression reduction in D97/D133 double KO cell lines, using qRT-PCR, normalized to U6 snRNA. (D)-(E) HEK293 cells were infected with pLV-EF1a-control (hashed circle), or pLV-EF1a expressing eMet-CAU (open square), a mixture of pLV-EF1a expressing 7 tRNAs: eMet-CAU, Arg-CCU, Gly-UCC, Ile-UAU, Lys-CUU, Sec-UCA, Trp-CCA (open triangle), or untreated (open circle). After selection with puromycin for 3 days, when WT un-infected cells have died, RNA expression (qRT-PCR) and cell growth were measured for 4 days. (F) For nuclear-encoded mRNAs in RNA-seq and ribo-seq from WT and D97 and D133 single KO HEK293 cells, ratios of expression levels were ranked. Then the usage of all 66 codons (standard 64 plus initiator Met and selenocysteine) were calculated for the top 10% (most up-regulated) and bottom 10% (most down-regulated) mRNAs and weighted by expression levels. The ratios were plotted in ranked order. A/U-ending codonsG/C ending codons. Asterisk (*): 3 stop codons. M and m: elongator and initiator methionine. U_uga: selenocysteine. This analysis shows that the elongator Met-AUG codon is only reduced on the translatome level, but not the transcriptome level. (G) Same as panel g. except that amino acid usage was calculated for RNA-seq and ribo-seq data. (H) Changes in codon usage frequencies were calculated and ratios KO/WT were plotted for RNA-seq and ribo-seq. The linear regression properties are at the bottom: equation, correlation coefficient r and statistical significance p. NN[GC]: G/C ending codons. NN[AU]: A/U ending codons. The inset ratios indicate ratios of average NN[GC] vs. average NN[AU] in RNA-seq and ribo-seq data. p values after the ratios are two-sided unpaired t-tests between the two codon groups.

FIG. 9A-C. Example GO analysis of codon usage. (A-B) Example gene ontology (GO) terms overrepresented in high and low Met_AUG codons, from the analysis of APPRIS collection of principal transcripts, among GO terms in c5.all.v2023.1.Hs.symbols.gmt. BP: biological processes. CC: cellular components. (N)ES: (normalized) enrichment score. nom p: nominal p values. (C) Gene specific codon usage (GSCU) values for Met AUG codon in the 5 complexes of oxidative phosphorylation, only showing nuclear-encoded mRNAs. Principal isoforms of human mRNAs in the APPRIS collection were used for calculation. Numbers in parentheses are the genes included in each complex. Not all components are enriched in Met.

FIG. 10A-D. Characterization of D133 KO mESC. (A) Genomic DNA PCR validation of the three clones of D133 KO mES cell lines. (B) Bright field view of WT and D133 KO TC1 mES and EBs at 4× and 20× magnifications. (C) Expression of Myh6 mRNA during the course od mES differentiation into CMs, plotted as Ct numbers relative to Atcb (upper panel), and in D133 KO #5 vs. WT cell line. p values are from two-sided t-tests. (D) Expression dynamics of snoRNAs and nuclear-encoded cytoplasmic tRNAs are measured in the RNA-seq data and shown in violin plus box plots. For each RNA, the WT mES value was set to 1. P values are from Wilcoxon signed rank test. (E) tRNA levels between D133 KO clone #5 and WT mES cells at different stages of differentiation. All nuclear-encoded tRNA genes were grouped by anticodons and then log 2 transformed ratios between KO and WT were normalized so that median=1 for each KO/WT comparison and then plotted. The primary mapped location on a tRNA gene was counted for all multi-mapped reads.

FIG. 11A-C. Differential gene expression analysis in D133 KO mES cells. (A) Expression differences between D133 KO and WT, and dynamics across ES, EB, and CM, for one-carbon metabolism mRNAs. Violin and box plots of the log transformed KO/WT ratios are summarized in the inset panel, showing the increased one-carbon metabolism activity in mES cells after D133 KO, which then returned to the same level as WT at the CM stage. (B) The one-carbon cycles redrawn from Ducker and Rabinowitz et al. 2017, where upregulated enzymes are highlighted. (C) Example downregulated GOBP term postsynaptic density assembly. Three biological replicates were used to report the standard deviations (error bars). P values indicate unpaired t-tests between each KO and WT mES cell line, unless otherwise noted.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001, or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units is also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2, 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps.

As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

“Embryonic stem cells”: abbreviated as ‘ES cells’ or ESC (or if of human origin ‘hES cells’ or ‘hESCs’) refers to stem cells that are derived from the inner cell mass of a blastocyst. The skilled person understands how to obtain such embryonic stem cells, for example as described by Chung (Chung et al., (2008) Stem Cell Lines, Vol 2(2): 113-117), which employs a technique that does not cause the destruction of the donor embryo(s). Various ESC lines are listed in the NIH Human Embryonic Stem Cell Registry. Pluripotent embryonic stem cells can be distinguished from other types of cells by the use of markers or lineage-specific markers including, but not limited to, Oct-4, Nanog, GCTM-2, SSEA3, and SSEA4.

“Induced pluripotent stem cell” or “iPSC”: These terms refer to pluripotent stem cells that are derived from a cell that is not a pluripotent stem cell (i.e., from a cell that is differentiated relative to a pluripotent stem cell). Induced pluripotent stem cell can be derived from multiple different cell types, including terminally differentiated cells. Induced pluripotent stem cell generally have an hESC cell-like morphology, growing as flat colonies with large nucleo-cytoplasmic ratios, defined borders and prominent nuclei. In addition, induced pluripotent stem cells may express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing induced pluripotent stem cells may be found in, for example, U.S. Patent Publication Nos. 2009/0047263, 2009/0068742, 2009/0191159, 2009/0227032, 2009/0246875, and 2009/0304646. To generate induced pluripotent stem cells, somatic cells may be provided with reprogramming factors (e.g. Oct4, SOX2. KLF4, MYC, Nanog, Lin28, etc.) known in the art to reprogram the somatic cells to become pluripotent stem cells (see, for example, Takahashi et. al, Cell. 2007 Nov. 30; 131(5):861-72; Takahashi et. al, Nat Protoc. 2007; 2(12):3081-9; Yu et. al, Science. 2007 Dec. 21:318(5858):1917-20. Epub 2007 Nov. 20).

“Pluripotency”: This term is generally understood by the skilled person and refers to an attribute of a (stem) cell that has the potential to differentiate into all cells constituting one or more tissues or organs, for example, any of the three germ layers: endoderm (e.g. interior stomach lining, gastrointestinal tract, the lungs), mesoderm (e.g. heart, muscle, bone, blood, urogenital tract), or ectoderm (e.g. epidermal tissues and nervous system).

“Pluripotent stem cell” or “PSC”: This is a stem cell capable of producing all cell types of the organism and can produce cells of the germ layers, e.g. endoderm, mesoderm, and ectoderm, of a mammal and encompasses at least pluripotent embryonic stem cells and induced pluripotent stem cells. Pluripotent stem cells can be obtained in different ways. Pluripotent embryonic stem cells may, for example, be obtained from the inner cell mass of an embryo. Induced pluripotent stem cells (iPSCs) may be derived from somatic cells. Pluripotent stem cells may also be in the form of an established cell line.

As used herein, the term “mesoderm” refers to one of the three germ layers that appears during early embryogenesis and which gives rise to various specialized cell types including blood cells of the circulatory system, muscles, the heart, the dermis, skeleton, and other supportive and connective tissues.

The terms “mesodermal cell lineage” and “mesodermal cell types” are used interchangeably herein and refer to mesodermally derived cells that have terminally differentiated and are readily identifiable as such. Such differentiated mesodermal cell types may or may not be proliferative. As described herein, mesodermal cell types include those adult cell types and cells of adult tissues derived from mesoderm that are well-known to the ordinary skilled artisan, including but not limited to, e.g., cardiac progenitor cells, endothelial progenitor cells, cardiomyocytes, skeletal muscle cells, smooth muscle cells, kidney cells, endothelial cells, skin cells, adrenal cortex cells, bone cells, white blood cells, and microglial cells; or are of an ectodermal lineage selected from: are neural stem cells, neurons, astrocytes, oligodendrocytes, or glial cells.

“Cardiomyocytes” or “cardiac myocytes”: This refers to any cardiomyocyte lineage cells and can be taken to apply to cells at any stage of cardiomyocyte ontogeny, unless otherwise specified. For example, cardiomyocytes may include both cardiomyocyte precursor or progenitor cells (i.e., cells that are capable, without dedifferentiation or reprogramming, of giving rise to progeny that include cardiomyocytes, e.g., immature cardiomyocytes or fetal cardiomyocytes) and mature cardiomyocytes (adult-like cardiomyocytes). Cardiomyocytes include atrial type cardiomyocytes, ventricular type cardiomyocytes, and nodal type cardiomyocytes and/or conducting system cardiomyocytes (see e.g., Maltsev et al, Mech Dev. 1993 November; 44(1):41-50 or Cardiac Regeneration using Stem Cells (10 Apr. 2013); Keiichi Fukuda, Shinsuke Yuasa CRC Press. ISBN 9781466578401). The cardiomyocyte progenitors, like the mature cardiomyocytes, may express markers typical of the cardiomyocyte lineage, including, without limitation, alpha actinin, cardiac troponin I (cTnl), cardiac troponin T (cTnT), sarcomeric myosin heavy chain (MHC), GATA-4, Nkx2.5, N-cadherin, 01-adrenoceptor (01-AR), ANF, the MEF-2 family of transcription factors, creatine kinase MB (CK-MB), myoglobin, or atrial natriuretic factor (ANF).

The terms “endodermal cell lineage” and “endodermal cell types” are used interchangeably herein and refer to endodermally derived cells that have terminally differentiated and are readily identifiable as such. Such differentiated endodermal cell types may or may not be proliferative. As described herein, mesodermal cell types include those adult cell types and cells of adult tissues derived from endoderm that are well-known to the ordinary skilled artisan, including but not limited to, e.g., exocrine epithelial cells, barrier cells, and hormone secreting cells. The exocrine epithelial cells include the Brunner's gland cell in duodenum, goblet cells in respiratory tract and digestive tract, pit cells, chief cells, parietal cells in stomach, pancreatic acinar cells, Paneth cell in small intestine, lung type II alveolar cells and rod cells in lung. Barrier cells include type I lung cells, gallbladder epithelial cells, centroacinar cells, intercalated duct cells and intestinal brush margin cells. There are four types of hormone secreting cells: intestinal endocrine cells, thyroid cells, parathyroid cells and islet cells. Intestinal endocrine cells include K cells, L cells, I cells, G cells, enterochromaffin cells, enterochromaffin-like cells, N cells, S cells, D cells and Mo cells. Thyroid cells include thyroid epithelial cells and parafollicular cells. Parathyroid cells include parathyroid main cells and eosinophils. Islet cells include Alpha cells, Beta cells, Delta cells, Epsilon cells and pp cells. Cells derived from ectoderm mainly include exocrine epithelial cells, hormone secreting cells, epithelial cells, nervous system cells and so on. Among them, exocrine cells include salivary gland mucus cells, salivary gland serous cells, Von Ebner's gland cell in tongue, mammary gland cells, lacrimal gland cells, earwax gland cells in ear, exocrine sweat gland dark cells, exocrine sweat gland bright cells, apocrine sweat gland cells, Gland of Moll cell in eyelid, adipose gland cells and Brunner's gland cell in duodenum. Hormone secreting cells include corticotropin cells, gonadotropin cells, prolactin cells, melanotropin cells, growth hormone cells and thyroid stimulating cells in the anterior and middle pituitary gland, large cell neurosecretory cells, small cell neurosecretory cells, chromaffin cells. Epithelial cells include keratinocytes, epidermal basal cells, melanocytes, medullary hair cells, cortical hair axons cells, epidermal hair axons cells, Huxley layer hair root sheath cells, outer root sheath hair cells, surface epithelial cells, basal cells, intercalated duct cells, striated tube cells, lactiferous duct cells and ameloblasts. Cells in the nervous system are divided into five categories: sensor cells, autonomic nerve cells, sensory organs and peripheral neuron supporting cells, central nervous system neurons and glial cells, lens cells. Sensor cells include cortical auditory inner hair cells, cortical auditory outer hair cells, basal cells of olfactory epithelium, cold sensitive primary sensory neurons, heat-sensitive primary sensory neurons, epidermal Merkel cells, olfactory receptor neurons, pain-sensitive primary sensory neurons, proprioceptive primary sensory neurons, tactile-sensitive primary sensory neurons, carotid somatic cytochemical receptor ball cells, outer hair cells of ear vestibular system, inner hair cells of ear vestibular system, taste receptor cells of taste buds, retinal photoreceptor cells, wherein retinal photoreceptor cells can be subdivided into photoreceptor rod cells, photoreceptor blue sensitive cone cells, photoreceptor green sensitive cone cells and photoreceptor red sensitive cone cells. Autonomic nerve cells include cholinergic nerve cells, adrenergic nerve cells and polypeptide nerve cells. Sertoli cells of sensory organs and peripheral neurons include intracortical column cells, extracortical column cells, intracortical finger cells, extracortical finger cells, cortical marginal cells, cortical Hensen cells, vestibular Sertoli cells, taste bud Sertoli cells, olfactory epithelial Sertoli cells, Schwann cells, satellite glial cells and intestinal glial cells. Neurons and glial cells in the central nervous system include neuronal cells, astrocytes, oligodendrocytes, ependymal cells, pituitary cells, wherein neurons cells can be divided into interneurons and principal cells, interneurons include basket cells, wheel cells, stellate cells, golgi cells, granulosa cells, Lugaro cells, unipolar brush cells, Martinotti cells, chandelier cells, Cajal-Retzius cells, Double-bouquet cells, glial cells, retinal horizontal cells, amacrine cells, spinal cord interneurons and Renshaw cells; principal cells include principal axis neurons, fork neurons, pyramidal cells, stellate cells, boundary cells, hairy cells, Purkinje cells and medium-sized spiny neurons, wherein pyramidal cells include position cells, location cells, velocity cells, direction identification cells and giant pyramidal cells. Lens cells include lens epithelial cells and lens fiber cells.

The cells derived from mesoderm mainly include metabolic and storage cells, secretory cells, barrier cells, extracellular stromal cells, contractile cells, blood and immune system cells, germ cells, trophoblast cells and interstitial tissue cells. Metabolic and storage cells include white adipocytes, brown adipocytes and liver adipocytes. Secretory cells include three types of adrenal cortex cells including adrenal cortical zona globularis cells producing mineralocorticoids, adrenal cortical fascicular cells producing glucocorticoids and adrenal cortical reticular zone cells producing androgen, ovarian follicular intimal cells, granular lutein cells, luteal cells, testicular interstitial cells, seminal vesicle cells, prostate cells, bulbar gland cells, pasteurian gland cells, urethral or periurethral gland cells, endometrial cells, paraglomerular cells, renal dense plaque cells, renal peripolar cells, renal mesangial cells. Barrier cells can be divided into three types: podocytes, proximal tubule brush margin cells, Henry's ring thin segment cells, renal distal tubule cells, main cells and intercalary cells in renal collecting duct cells, and transitional epithelial cells in urinary system; ductal cells, efferent ductal cells, epididymal principal cells and epididymal basal cells in reproductive system; endothelial cells in the circulatory system. Extracellular stromal cells include ear vestibular semicircular canal epithelial cells, cortical interdentate epithelial cells, loose connective tissue fibroblasts, corneal fibroblasts, tendon fibroblasts, bone marrow reticular fibroblasts, other non-epithelial fibroblasts, pericytes, such as hepatic stellate cells, intervertebral disc nucleus pulposus cells, hyaline chondrocytes, fibrochondrocytes, elastic chondrocytes, osteoblasts, bone progenitor cells, vitreous clear cells, extraaural lymphatic space stellate cells, pancreatic stellate cells. Contractile cells include six types of skeletal muscle cells including red skeletal muscle cells, white skeletal muscle cells, intermediate skeletal muscle cells, muscular spindle nucleus bag cells, muscular spindle nucleus chain cells and muscular satellite cells, three kinds of cardiomyocytes including myocardial cells, sinoatrial node cells and Purkinje fiber cells, smooth muscle cells, iris myoepithelial cells and exocrine gland myoepithelial cells. Blood and immune system cells includes red blood cells, megakaryocytes, platelets, monocytes, macrophages in connective tissue, langerhans cells in the epidermis, osteoclasts, dendritic cells, microglia, neutrophils, eosinophils, basophils, hybridoma cells, mast cells, helper T cells, suppressor T cells, cytotoxic T cells, natural killer T cells, B cells, natural killer cells, reticular cells, stem cells and progenitor cells of blood and immune system. Germ cells include oogonia/oocytes, spermatocytes, spermatogonia, spermatocytes and sperm. Trophoblasts include granulosa cells in ovary, Sertoli cells in testis and epithelial reticular cells. Interstitial tissue cells include interstitial tissue kidney cells.

“Differentiating” and “differentiation”: these terms, in the context of living cells, relate to progression of a cell further down the developmental pathway. A “differentiated cell” is a cell that has progressed further down the developmental pathway than the cell it is being compared with; differentiation is the process of progression. Human pluripotent stem cells can differentiate into lineage-restricted progenitor cells (cells that, like a stem cell, have a tendency to differentiate into a specific type of cell, but are already more differentiated than a stem cell and are pushed to eventually differentiate into its end-stage cell; e.g. endoderm, mesoderm and ectoderm), which in turn can differentiate into further restricted cells (e.g., cardiomyocyte progenitors, neuronal cell progenitors), which can differentiate into terminally differentiated cells (e.g., cardiomyocytes (e.g. atrial and ventricular cardiomyocytes) or neurons). Differentiation is controlled by the interaction of a cell's genes with the physical and chemical conditions outside the cell, usually through signaling pathways involving proteins embedded in the cell surface. In the present invention, “differentiation” is the biological process whereby an unspecialized human pluripotent stem cell (population) acquires the features of a specialized cell such as a cardiomyocyte, particularly cardiomyocytes having an atrial phenotype, under controlled conditions in in vitro culture. The human pluripotent stem cells may be exposed to the culture media compositions and methods of the invention so as to promote differentiation of the human pluripotent stem cells into cardiomyocytes, particularly cardiomyocytes having an atrial phenotype. Cardiac differentiation in general can be detected by the use of markers selected from, but not limited to, alpha actinin, NKX2-5, GATA4, myosin heavy chain, myosin light chain, troponin, and tropomyosin (Burridge et al., (2012) Stem Cell, Vol. 10(1):16-28; U.S. Patent Pub. No. 2013/0029368). Within the context of the current invention, human pluripotent stem cell population are differentiated toward cardiomyocytes, preferably cardiomyocytes having or displaying an atrial phenotype, for example as witnessed by the presence of COUP-TFI and/or II in or on the cell or up-regulation of COUP-TFI and/or II levels in pluripotent stem cell-derived cardiomyocytes obtained by the methods of the present invention.

“Undifferentiated”: A stem cell that has not developed a characteristic of a more specialized cell is an undifferentiated cell. As will be recognized by one of skill in the art, the terms “undifferentiated” and “differentiated” are relative with respect to each other. A cell that is ‘differentiated’ has a characteristic of a more specialized cell. Differentiated and undifferentiated cells are distinguished from each other by several well-established criteria, including morphological characteristics such as relative size and shape, ratio of nuclear volume to cytoplasmic volume; and expression characteristics such as detectable presence of known (gene) markers of differentiation.

As used herein, the term “complementary” refers to a double-stranded DNA or RNA strand that consists of two complementary strands of base pairs. Complementary binding occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA.

In this example, the sequence 5′-ATCG-3′ is the reverse complement of 3′-TAGC-5′. The term “nucleic acid” refers to deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein.

As used herein, the term “snoRNA” refers to small nucleolar RNAs. snoRNA guide chemical modifications of other RNAs, such as rRNAs, tRNAs, and snRNAs; these small non-coding RNAs fall into two classes, one of about 60-90 nucleotides long (“box C/D”), and another of about 120-140 nucleotides long (“box H/ACA”) (Dupuis-Sandoval et al., 2015. Rev RNA, 6: 381-397).

### Embodiments of the Invention

The dynamic balance between tRNA supply and codon usage demand is a fundamental principle in the cellular translation economy. However, the regulation and functional consequences of this balance remain unclear. Here we use PARIS2 interactome capture, structure modeling, conservation analysis, RNA-protein interaction analysis, and modification mapping to reveal the targets of hundreds of snoRNAs, many of which were previously considered as orphans. We discovered a snoRNA-tRNA interaction network that is required for global tRNA modifications, including 2′-O-methylation and others. Loss of FBL, the snoRNA-guided 2′-O-methyltransferase, induces global upregulation of tRNA fragments, a large group of regulatory RNAs. In particular, the snoRNAs D97/D133 guide the 2′-O-methylation of multiple tRNAs, especially for the amino acid methionine (Met), a protein-intrinsic antioxidant. Loss of D97/D133 snoRNAs in human HEK293 cells reduced target tRNA levels and induced codon adaptation of the transcriptome and translatome. Both D97/D133 single and double knockouts in HEK293 cells suppress Met-enriched proliferation-related gene expression programs, including, translation, splicing and mitochondrial energy metabolism, and promotes Met-depleted programs related to development, differentiation, and morphogenesis. In a mouse embryonic stem cell model of development, knockdown and knockout of D97/D133 promote differentiation to mesoderm and endoderm fates, such as cardiomyocytes, without compromising pluripotency, consistent with the enhanced development-related gene expression programs in human cells. This work solves a decades-old mystery about orphan snoRNAs and reveals a new function of snoRNAs in controlling the codon-biased dichotomous cellular states of proliferation and development.

Accordingly, the disclosure provides for methods of inducing cell differentiation from stem cells, and in particular, differentiation into cardiomyocytes, and composition of differentiated cell produced using the methods described herein. In some embodiments, a method for promoting or inducing stem cell differentiation into a mesoderm cell lineage or an endoderm cell lineage comprises contacting a stem cell with an agent that reduces or eliminates expression and/or activity of one or more endogenous small nuclear ribonucleic acid (snoRNA) molecules, wherein the snoRNA molecules comprise SNORD97 and/or SNORD133.

In some embodiments, the agent is an aptamer, short interfering RNA (siRNA), micro-RNA (miRNA), short hairpin RNA (shRNA), DNA, an antisense polynucleotide, a CRISPR/Cas9 system, or a chemical compound.

Interfering RNA (which may be interchangeably referred to as RNAi or an interfering RNA sequence) refers to double-stranded RNA that is capable of silencing, reducing, or inhibiting expression of a target gene by any mechanism of action now known or yet to be disclosed. For example, RNAi may act by mediating the degradation of mRNAs which are complementary to the sequence of the RNAi when the RNAi is in the same cell as the target gene. As used herein, RNAi may refer to double-stranded RNA formed by two complementary RNA strands or by a single, self-complementary strand. RNAi may be substantially or completely complementary to the target mRNA or may comprise one or more mismatches upon alignment to the target mRNA. The sequence of the interfering RNA may correspond to the full-length target mRNA, or any subsequence thereof.

Generally speaking, RNAi is a multistep process. In a first step, there is cleavage of large dsRNAs into 21-23 ribonucleotides-long double-stranded effector molecules called “small interfering RNAs” or “short interfering RNAs” (siRNAs). These siRNAs duplexes then associate with an endonuclease-containing complex, known as RNA-induced silencing complex (RISC). The RISC specifically recognizes and cleaves the endogenous mRNAs/RNAs containing a sequence complementary to one of the siRNA strands. One of the strands of the double-stranded siRNA molecule (the “guide” strand) comprises a nucleotide sequence that is complementary to a nucleotide sequence of the target gene, or a portion thereof, and the second strand of the double-stranded siRNA molecule (the passenger” strand) comprises a nucleotide sequence substantially similar to the nucleotide sequence of the target gene, or a portion thereof.

In more particular embodiments, siRNAs comprise a duplex, or double-stranded region, of about 18-25 nucleotides long. Often, siRNAs contain from about two to four unpaired nucleotides at the 3′ end of each strand. At least a portion of one strand of the duplex or double-stranded region of a siRNA is substantially homologous to or substantially complementary to a target sequence within the gene product (i.e., RNA) molecule as herein defined. The strand complementary to a target RNA molecule is the “antisense guide strand”, the strand homologous to the target RNA molecule is the “sense passenger strand” (which is also complementary to the siRNA antisense guide strand). siRNAs may also be contained within structures such as miRNA and shRNA that have additional sequences such as loops, linking sequences, as well as stems and other folded structures.

As noted above, RNAi includes small-interfering RNA, which, herein, may interchangeably be referred to as siRNA. siRNA is described for example in U.S. Pat. Nos. 9,328,347; 9,328,348; 9,289,514; 9,289,505; and 9,273,312. A siRNA may be any interfering RNA with a duplex length of about 15-60, 15-50, or 15-40 nucleotides in length, more typically about 15-30, 15-25, or 18-23 nucleotides in length. Each complementary sequence of the double-stranded siRNA may be 15-60, 15-50, 15-40, 15-30, 15-25, or 18-23 nucleotides in length, but other noncomplementary sequences may be present. For example, siRNA duplexes may comprise 3′ overhangs of 1 to 4 or more nucleotides and/or 5′ phosphate termini comprising 1 to 4 or more nucleotides. A siRNA may be synthesized in any of a number of conformations. One of ordinary skill in the art would recognize the type of siRNA conformation to be used for a particular purpose. Examples of siRNA conformations include, but need not be limited to, a double-stranded polynucleotide molecule assembled from two separate stranded molecules, wherein one strand is the sense strand and the other is the complementary antisense strand; a double-stranded polynucleotide molecule assembled from a single-stranded molecule, where the sense and antisense regions are linked by a nucleic acid-based or non-nucleic acid-based linker; a double-stranded polynucleotide molecule with a hairpin secondary structure having complementary sense and antisense regions; or a circular single-stranded polynucleotide molecule with two or more loop structures and a stem having self-complementary sense and antisense regions. In the case of the circular polynucleotide, the polynucleotide may be processed either in vivo or in vitro to generate an active double-stranded siRNA molecule.

SiRNA can be chemically synthesized, may be encoded by a plasmid and transcribed, or may be vectored by a virus engineered to express the siRNA. A siRNA may be a single stranded molecule with complementary sequences that self-hybridize into duplexes with hairpin loops. siRNA can also be generated by cleavage of parent dsRNA through the use of an appropriate enzyme such as E. coli RNase III or Dicer (Yang et al., Proc. Natl. Acad. Sci. USA 99, 9942-9947 (2002); Calegari et al., Proc. Natl. Acad. Sci. USA 99, 14236-14240 (2002); Byrom et al, Ambion Tech Notes 10, 4-6 (2003); Kawasaki et al, Nucleic Acids Res 31, 981-987 (2003); and Knight et al., Science 293, 2269-2271 (2001). A parent dsRNA may be any double stranded RNA duplex from which a siRNA may be produced, such as a full or partial mRNA transcript.

A mismatch motif may be any portion of a siRNA sequence that is not 100% complementary to its target sequence. A siRNA may have zero, one, two, or three or more mismatch regions. The mismatch regions may be contiguous or may be separated by any number of complementary nucleotides. The mismatch motifs or regions may comprise a single nucleotide or may comprise two or more consecutive nucleotides.

SiRNA molecules can be provided in several forms including, e.g., as one or more isolated siRNA duplexes, as longer double-stranded RNA (dsRNA), or as siRNA or dsRNA transcribed from a transcriptional cassette in a DNA plasmid. The siRNA sequences may have overhangs (as 3′ or 5′ overhangs as described in Elbashir et al, Genes Dev 15, 188 (2001), the content of each of which is incorporated by reference herein in its entirety) or may lack overhangs (i.e., have blunt ends).

One or more DNA plasmids encoding one or more siRNA templates may be used to provide siRNA. siRNA can be transcribed as sequences that automatically fold into duplexes with hairpin loops from DNA templates in plasmids having RNA polymerase III transcriptional units, for example, based on the naturally occurring transcription units for small nuclear RNA U6 or human RNase P, RNAse H1 (Brummelkamp et al, Science 296, 550 (2002); Donze et al, Nucleic Acids Res 30, e46 (2002); Paddison et al, Genes Dev 16, 948 (2002)). Typically, a transcriptional unit or cassette will contain an RNA transcript promoter sequence, such as an H1-RNA or a U6 promoter, operably linked to a template for transcription of a desired siRNA sequence and a termination sequence, comprised of 2-3 uridine residues and a polythymidine (T5) sequence (polyadenylation signal). The selected promoter can provide for constitutive or inducible transcription. Compositions and methods for DNA-directed transcription of RNA interference molecules are described in detail in U.S. Pat. No. 6,573,099. The transcriptional unit is incorporated into a plasmid or DNA vector from which the interfering RNA is transcribed. Plasmids suitable for in vivo delivery of genetic material for therapeutic purposes are described in detail in U.S. Pat. Nos. 5,962,428 and 5,910,488. The selected plasmid can provide for transient or stable delivery of a nucleic acid to a target cell. It will be apparent to those of skill in the art that plasmids originally designed to express desired gene sequences can be modified to contain a transcriptional unit cassette for transcription of siRNA.

Methods for isolating RNA, synthesizing RNA, hybridizing nucleic acids, making and screening cDNA libraries, and performing PCR are well known in the art (see, e.g., Gubler and Hoffman, Gene 25, 263-269 (1983); Sambrook and Russell, Molecular Cloning: A Laboratory Manual, Cold Spring Harbor Laboratory Press, Cold Spring Harbor N.Y., (2001)) as are PCR methods (see, U.S. Pat. Nos. 4,683,195 and 4,683,202; PCR Protocols: A Guide to Methods and Applications, Innis et al, eds, (1990).

A siRNA molecule may be chemically synthesized. In one example of chemical synthesis, a single-stranded nucleic acid that includes the siRNA duplex sequence can be synthesized using any of a variety of techniques known in the art, such as those described in Usman et al, J Am Chem Soc, 109, 7845 (1987); Scaringe et al, Nucl Acids Res, 18, 5433 (1990); Wincott et al, Nucl Acids Res, 23, 2677-2684 (1995); and Wincott et al, Methods Mol Bio 74, 59 (1997). Synthesis of the single-stranded nucleic acid makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer (Thermo Fisher Scientific, Waltham, Mass.) using a 0.2 micromolar scale protocol with a 2.5 min coupling step for 2′-O-methylated nucleotides. Alternatively, syntheses at the 0.2 micromolar scale can be performed on a 96-well plate synthesizer from Thermo Fisher Scientific. However, larger or smaller scale synthesis are also encompassed by the invention, including any method of synthesis now known or yet to be disclosed. Suitable reagents for synthesis of siRNA single-stranded molecules, methods for RNA deprotection, and methods for RNA purification are known to those of skill in the art.

In certain embodiments, siRNA can be synthesized via a tandem synthesis technique, wherein both strands are synthesized as a single continuous fragment or strand separated by a linker that is subsequently cleaved to provide separate fragments or strands that hybridize to form a siRNA duplex. Linkers may be any linker, including a polynucleotide linker or a non-nucleotide linker. The tandem synthesis of siRNA can be readily adapted to both multiwell/multiplate synthesis platforms as well as large scale synthesis platforms employing batch reactors, synthesis columns, and the like. In some embodiments, siRNA can be assembled from two distinct single-stranded molecules, wherein one strand includes the sense strand and the other includes the antisense strand of the siRNA. For example, each strand can be synthesized separately and joined together by hybridization or ligation following synthesis and/or deprotection. Either the sense or the antisense strand may contain additional nucleotides that are not complementary to one another and do not form a double stranded siRNA. In certain instances, siRNA molecules can be synthesized as a single continuous fragment, where the self-complementary sense and antisense regions hybridize to form a siRNA duplex having hairpin secondary structure.

A siRNA molecule may comprise a duplex having two complementary strands that form a double-stranded region with least one modified nucleotide in the double-stranded region. The modified nucleotide may be on one strand or both. If the modified nucleotide is present on both strands, it may be in the same or different positions on each strand. A modified siRNA may be less immunostimulatory than a corresponding unmodified siRNA sequence but retains the capability of silencing the expression of a target sequence.

Examples of modified nucleotides suitable for use in the present invention include, but are not limited to, ribonucleotides having a 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, or 2′-C-allyl group. Modified nucleotides having a conformation such as those described in the art, for example in Sanger, Principles of Nucleic Acid Structure, Springer-Verlag Ed. (1984), incorporated by reference herein in its entirety, are also suitable for use in siRNA molecules. Other modified nucleotides include, without limitation: locked nucleic acid (LNA) nucleotides, G-clamp nucleotides, or nucleotide base analogs. LNA nucleotides include but need not be limited to 2′-O,4′-C-methylene-(D-ribofuranosyl)nucleotides), 2′-O-(2-methoxyethyl) (MOE) nucleotides, 2′-methyl-thio-ethyl nucleotides, 2′-deoxy-2′-fluoro (2′F) nucleotides, 2′-deoxy-2′-chloro (2Cl) nucleotides, and 2′-azido nucleotides. A G-clamp nucleotide refers to a modified cytosine analog wherein the modifications confer the ability to hydrogen bond both Watson-Crick and Hoogsteen faces of a complementary guanine nucleotide within a duplex (Lin et al, J Am Chem Soc, 120, 8531-8532 (1998)).Nucleotide base analogs include for example, C-phenyl, C-naphthyl, other aromatic derivatives, inosine, azole carboxamides, and nitroazole derivatives such as 3-nitropyrrole, 4-nitroindole, 5-nitroindole, and 6-nitroindole (Loakes et al., Nucl Acids Res, 29, 2437-2447 (2001)).

A siRNA molecule may comprise one or more non-nucleotides in one or both strands of the siRNA. A non-nucleotide may be any subunit, functional group, or other molecular entity capable of being incorporated into a nucleic acid chain in the place of one or more nucleotide units that is not or does not comprise a commonly recognized nucleotide base such as adenosine, guanine, cytosine, uracil, or thymine, such as a sugar or phosphate.

Chemical modification of siRNA may comprise attaching a conjugate to a siRNA molecule. The conjugate can be attached at the 5′- and/or the 3′-end of the sense and/or the antisense strand of the siRNA via a covalent attachment such as a nucleic acid or non-nucleic acid linker. The conjugate can be attached to the siRNA through a carbamate group or other linking group (see, e.g., U.S. Patent Publication Nos. 2005/0074771, 2005/0043219, and 2005/0158727). A conjugate may be added to siRNA for any of a number of purposes. For example, the conjugate may be a molecular entity that facilitates the delivery of siRNA into a cell or may be a molecule that comprises a drug or label. Examples of conjugate molecules suitable for attachment to siRNA of the present invention include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 2003/0130186, 2004/0110296, and 2004/0249178; U.S. Pat. No. 6,753,423). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 2005/0119470 and 2005/0107325. Other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules described in U.S. Patent Publication No. 2005/0153337. Additional examples of conjugate molecules include a hydrophobic group, a membrane active compound, a cell penetrating compound, a cell targeting signal, an interaction modifier, or a steric stabilizer as described in U.S. Patent Publication No. 2004/0167090. Further examples include the conjugate molecules described in U.S. Patent Publication No. 2005/0239739.

In other embodiments, strands of a double-stranded interfering RNA (e.g., siRNA) may be connected to form a hairpin or stem-loop structure (e.g., shRNA). Thus, as mentioned above the agent also may be a short hairpin RNA (shRNA).

According to other embodiments, an agent may comprise a micro-RNA (miRNA). miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA. The pre-miRNA is a substrate for a form of dicer that removes the miRNA duplex from the precursor, after which, similarly to siRNAs, the duplex can be taken into the RISC complex. Unlike, siRNAs, miRNAs bind to transcript sequences with only partial complementarity and usually repress translation without affecting steady-state RNA levels. Both miRNAs and siRNAs are processed by Dicer and associate with components of the RNA-induced silencing complex (RISC). (See, for example, Michaels et al., Nature Communications, 19:818. 2019) An agent may comprise a nucleic acid agent that may comprise at least one shRNA molecule. In more particular embodiments, such shRNA may comprise a nucleic acid sequence complementary at least in part to a target snoRNA, and in particular, SNORD97 and SNORD133. The term “shRNA”, as used herein, refers to an RNA agent having a stem-loop structure, comprising a first and second region of complementary sequence. The degree of complementarity and orientation of the regions being sufficient such that base pairing occurs between the regions. The first and second regions being joined by a loop region, the loop resulting from a lack of base pairing between nucleotides (or nucleotide analogs) within the loop region. Some of the nucleotides in the loop can be involved in base-pair interactions with other nucleotides in the loop.

In some specific embodiments, the shRNA may comprise a sequence complementary to the target snoRNA, and in particular, SNORD97 and SNORD133, having a length of between about 5 to 50 nucleotides, specifically, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 46, 37, 38, 39, 40, 41, 42, 43, 44, 45, 46, 47, 48, 49, 50 or more nucleotides. In more specific embodiments, the complementary sequence may be in a length ranging between 9 to 29 nucleotides.

In some embodiments, the agent comprises one or more of a short interfering RNA (siRNA), a micro-RNA (miRNA), and a short hairpin RNA (shRNA) that specifically binds to at least a portion of any one of SEQ ID NO: 1 to SEQ ID NO: 4. In some embodiments, the agent comprises one or more of a short interfering RNA (siRNA), a micro-RNA (miRNA), and a short hairpin RNA (shRNA) that is complementary to at least a portion of any one of SEQ ID NO: 1 to SEQ ID NO: 4. In other embodiments, the agent comprises siRNA having a nucleotide sequences at least about 85%, at least about 90%, at least about 95%, at least about 96%, at least about 97%, at least about 98%, at least about 99%, or 100% identical to any one of SEQ ID NO: 7 to SEQ ID NO: 14.

In some embodiments, target snoRNAs may be edited to reduce, suppress, or eliminate expression of said snoRNA gene using clustered regularly interspaced short palindromic repeats (CRISPR) and CRISPR-associated (Cas) proteins as described, for example, in U.S. Pat. Nos. 10,266,850, 10,227,611, 10,000,772, and 10,113,167, and U.S. Patent Pub. No. 20190134227.

In general, the term “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR system may comprise, for example, CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains). In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

In some embodiments, a Cas nuclease and guide RNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr-mate sequence (optionally operably linked to the guide sequence), and a tracr sequence embedded within one or more intron sequences (e.g., each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of the CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to the cell.

In some embodiments, a vector comprises a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9.

In some embodiments the CRISPR enzyme is Cas9 and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce non-homologous end joining.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR complex to a target sequence may be assessed by any suitable assay. For example, the components of the CRISPR system sufficient to form the CRISPR complex, including the guide sequence to be tested, may be provided to the cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of the CRISPR complex, including the guide sequence to be tested and a control guide sequence different from the test guide sequence, and comparing binding or rate of cleavage at the target sequence between the test and control guide sequence reactions.

A guide sequence may be selected to target any target sequence. In some embodiments, the target sequence is a sequence within a genome of a cell. Exemplary target sequences include those that are unique in the target genome. In some embodiments, a guide sequence is selected to reduce the degree of secondary structure within the guide sequence. Secondary structure may be determined by any suitable polynucleotide folding algorithm.

In general, a tracr-mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences.

Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In some aspects, loop forming sequences for use in hairpin structures are four nucleotides in length and have the sequence GAAA. However, longer or shorter loop sequences may be used, as well as alternative sequences. In some embodiments, the sequences include a nucleotide triplet (for example, AAA), and an additional nucleotide (for example C or G). Examples of loop forming sequences include CAAA and AAAG. In some embodiments, the transcript or transcribed polynucleotide sequence has at least two or more hairpins. In some embodiments, the transcript has two, three, four or five hairpins. In a further embodiment, the transcript has at most five hairpins. In some embodiments, the single transcript further includes a transcription termination sequence, such as a polyT sequence, for example six T nucleotides.

In some embodiments, the agent comprises a CRISPR/Cas9 system that specifically targets at least a portion of human fibrillarin gene (FBL) (GenBank Accession No. NC_000019.10) or at least a portion of human dyskerin pseudouridine synthase 1 (DKC1) (GenBank Accession No. NG_009780.1). In some embodiments, the CRISPR/Cas9 system uses a guide RNA according to any one of SEQ ID NO: 15 to SEQ ID NO: 20. In other embodiments, the agent comprises a CRISPR/Cas9 system that specifically targets at least a portion of SEQ ID NO: 1 and/or SEQ ID NO: 3.

In some embodiments, the agent is a synthetic antisense polynucleotide that specifically binds to at least a portion of any one of SEQ ID NO: 1 to SEQ ID NO: 4. In some embodiments, the antisense oligomer may include one or more modified bases such as, but not limited to, 2′-O-methoxy-ethyl bases (2′-MOE) (e.g., 2-MethoxyEthoxy Adenosine, 2-MethoxyEthoxy methylcytidine, 2-MethoxyEthoxy guanosine, 2-MethoxyEthoxy thymidine), 2′-O-methyl RNA bases, and fluorinated bases (e.g., fluorinated cytidine, fluorinated uridine, fluorinated adenosine, fluorinated guanosine). Other modified bases include, for example, puromycin, 8-oxo deoxyguanosine, N6-methyl-2′-deoxyadenosine, 5-bromo-deoxyuridine, deoxyuridine, 2,6-diaminopurine, dideoxycytidine, deoxyinosine, hydroxymethyl deoxycytidine, 5-methyl deoxycytidine, 5-nitroindole, 5-hydroxybutynl-2′-deoxyuridine, and 8-aza-7-deazaguanosine.

In some embodiments, the antisense polynucleotides comprise a central DNA or RNA segment flanked by modified RNA wings. In some embodiments, the central region of 10-12 DNA or RNA bases with 4-5 modified RNA bases on both sides of the central region. Each wing consists of 4-5 RNA bases, all or most of which are modified RNA bases, e.g., in which each modified RNA base is selected from the group consisting of 2′-O-methoxyethyl RNA and 2′-O-methyl RNA. A modified RNA base may include a substitution on a 2′ hydroxyl group of a ribose sugar. A 2′-O-Methoxyethyl (“2′-MOE”) modified sugar may be included in an RNA base.

In some embodiments, an antisense polynucleotide that specially binds to SNORD97 comprises the sequence: /52MOErC/*/i2MOErA/*/i2MOErT/*/i2MOErA/*/i2MOErT/*C*T*C*A*T*A*A*T*C*T*/i2MOErT/*/i2MOErC/*/i2MOErG/*/i2MOErC/*/32MOErT/(SEQ ID NO: 30). In some embodiments, an antisense polynucleotide that specially binds to SNORD133 comprises the sequence /52MOErT/*/i2MOErC/*/i2MOErA/*/i2MOErG/*/i2MOErA/*T*C*T*C*A*T*A*A*T*C*/i2MOErT/*/i2MOErT/*/i2MOErA/*/i2MOErC/*/3 2MOErC/(SEQ ID NO: 31), where 52MOEr is 5′ 2′-O-methoxyethyl RNA, 32MOEr is 3′ 2′-O-methoxyethyl RNA, i2MOEr is internal 2′-O-methoxyethyle RNA, and * denotes a phosphorothioate linkage.

In some embodiments, the linkages between bases may be all phosphorothioate or a mixture of phosphorothioate and phosphodiester bonds. Modified chemical bases are known in the art and are described, for example, at idtdna.com/site/catalog/modifications and in U.S. Patent Publication No. 2022/0259601 to Fink et al.

In some embodiments, the modified nucleotides may be independently selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide, and a 2′-O-(N-methylacetamide) modified nucleotide, and combinations thereof.

In some embodiments, specific portions of SNORD97 that may be targeted by an agent, for example, an antisense polynucleotide or siRNA, include atgtccagcgtcct (SEQ ID NO: 21) and tgagcgaagattatgagatatgagggcaa (SEQ ID NO: 22). In some embodiments, specific portions of SNORD133 that may be targeted by an agent, for example, an antisense polynucleotide or siRNA include gtgatga, tagaga, ataggactaactttc (SEQ ID NO: 23), attgttaagtcc (SEQ ID NO: 24), ggtaagattatgagatctga (SEQ ID NO: 25). In some embodiments, any one of SEQ ID NO: 21 to SEQ ID NO: 25 may be flanked on both the 5′ and 3′ end by about 5 to about 10 modified bases as discussed herein. In some embodiments, the modified base is selected from the group consisting of 5′ 2′-O-methoxyethyl RNA, 3′ 2′-O-methoxyethyl RNA, internal 2′-O-methoxyethyle RNA, and optionally may include one or more phosphorothioate linkages.

In some embodiments, the nitrogenous bases of the ASO may be naturally occurring nucleobases such as adenine, guanine, cytosine, thymidine, uracil, xanthine and hypoxanthine, as well as non-naturally occurring variants, such as substituted purine or substituted pyrimidine, such as nucleobases selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′-thio-thymine, inosine, diaminopurine, 6-aminopurine, 2-aminopurine, 2,6-diaminopurine and 2-chloro-6-aminopurine.

In some embodiments, the stem cells are induced into a cell of the mesoderm cell lineage such as, but not limited to, a cardiac cell. In some embodiments, the cardiac cell is selected from the group consisting of a cardiomyocyte, nodal cardiomyocyte, conducting cardiomyocyte, working cardiomyocyte, cardiomyocyte precursor, cardiomyocyte progenitor cell, cardiac stem cell, and cardiac muscle cell. Preferably, the cardiac cell comprises a cardiomyocyte.

The disclosure also provides for an isolated cardiac cell differentiated from a stem cell, wherein endogenous SNORD97 and/or SNORD133 gene activity of the isolated cardiac cell is i) eliminated or ii) reduced compared to a reference cell.

In some embodiments, the isolated cardiac cell is selected from the group consisting of a cardiomyocyte, nodal cardiomyocyte, conducting cardiomyocyte, working cardiomyocyte, cardiomyocyte precursor, cardiomyocyte progenitor cell, cardiac stem cell, and cardiac muscle cell. Methods of culturing cardiomyocytes are known in the art and described, for example, by Spater et al., Development (2014) 141 (23): 4418-4431.

The disclosure also provides for a composition comprising in vitro differentiated stem cells, wherein the in vitro differentiated stem cells are genetically modified to eliminate or have decreased expression and/or activity of one or more small nuclear ribonucleic acid (snoRNA) molecules, wherein the snoRNA comprises SNORD97 and SNORD133.

In some embodiments, the in vitro differentiated stem cells comprise cardiomyocytes. In some embodiments, the in vitro differentiated stem cells are derived from human embryonic stem cells or human induced pluripotent stem cells (iPSCs). Methods of culturing stem cell are known in the art and described, for example, by U.S. Patent Publication No. 2009/0275132 to Hattori et al., and 2003/0022367 to Xu et al.

Also provided are methods of treating a cardiac disease comprising administering to a subject in need thereof an effective amount of the composition or a cardiomyocyte produced according to the method disclosed herein. In some embodiments, the cardiac disease comprises pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.

### Results and Discussion.

Global Discovery of snoRNA Targets Using PARIS2 and dRMS.

To discover snoRNA targets, we applied PARIS2 to total RNA, chromatin associated RNA and antisense-oligo enriched snoRNAs, in human cell lines and induced pluripotent stem cell (iPS)-derived lineages. In PARIS2, psoralen crosslinking of RNA duplexes, proximity ligation and sequencing reveal transcriptome-wide RNA interactions. Specifically, cells were first crosslinked with psoralen, and then total RNA or chromatin-associated RNA were extracted and fragmented for PARIS2 library preparation. Alternatively, 46 snoRNA families, including 36 orphans, were enriched from the crosslinked total RNA by biotinylated antisense oligos for PARIS2 experiments. Together, these three approaches allowed us to discover the targets of a broader group of snoRNAs with higher sensitivity.

To validate these interactions, we employed multiple alternative approaches, including RiboMeth-seq (RMS) to map 2′-O-methylation (Erales et al., Proc Natl Acad Sci USA 114, 12934-12939 (2017); Yi et al., Nat Cell Biol 23, 341-354 (2021)), and CLIP to map protein-RNA interactions and protein-bound RNA-RNA interactions (Granneman et al., Proc Natl Acad Sci USA 106, 9613-9618 (2009)). The commonly used RMS hydrolyzes RNA at high pH, where 2′-O-methylation protection of the phosphodiester bond is detected by sequencing (Galvanin et al., Methods Mol Biol 1870, 273-295 (2019); Marchand et al., Nucleic Acids Res 44, e135 (2016); Birkedal et al., Angew Chem Int Ed Engl 54, 451-455 (2015)). However, stable RNA structures and dense modifications strongly skew the fragmentation and reverse transcription, impeding its application to many ncRNAs. We developed a denatured RMS (dRMS) method, where stronger denaturation in the presence of 95% DMSO during fragmentation increased the efficiency, uniformity of RNA fragmentation, and detection efficiency.

PARIS2 experiments revealed thousands of target sites for hundreds of snoRNAs, including rRNAs, snRNAs, nearly all nuclear-encoded tRNAs, and many other ncRNAs (n=7531 interactions after CRSSANT clustering). C/D snoRNA targets were captured more efficiently than H/ACA snoRNA targets, therefore, we focused on C/D snoRNAs for initial validation. PARIS2 captured significant fractions of PLEXY-predicted low energy snoRNA-rRNA interactions and known targets in published databases at various minimal free energy (MFE) cutoff. Known interactions are ranked among the top PARIS-derived contacts. EZH2 is not only a transcription repressor and lysine methyltransferase, but also a chaperone that facilitates the assembly of C/D box snoRNAs by interacting with FBL. Out of 98 known rRNA Nm sites in snoRNABase, the vast majority have reduced Nm levels upon disruption of snoRNPs by both FBL and EZH2 knockdown (KD), and PARIS2 captured 95 of them. For known Nm sites in rRNAs, guide snoRNAs were either unknown or only predicted in the snoAtlas database (n=6), which were all discovered by PARIS2 (n=98 for known Nm sites). De novo discovery of potential sites where Nm levels are reduced after FBL KD also revealed previously unknown sites, a subset of which are supported by PARIS2 (n=13, n=121 for de novo determined Nm sites). For example, the D′ guide of the orphan D101 is highly conserved in animals and plants. PARIS2 and structure modeling revealed a new target at 28S Gm3628. FBL is essential for cell survival, therefore only partial KD is possible, leading to modest reduction of methylation level at G3628, consistent with most other Nm sites on rRNAs. Similarly, analysis of published RMS data confirmed the reduction of G3628 methylation level after EZH2 KD.

Combining PARIS2 and dRMS, we validated known and further discovered multiple new Nm sites on several small RNAs, including spliceosomal snRNAs U1, U2 and U6, 7SL in the signal recognition particle, and snoRNAs. Together, these studies confirmed the accuracy of PARIS2 and dRMS and revealed by far the largest numbers of snoRNA targets across multiple ncRNA types.

A Global and Conserved snoRNA-tRNA Interaction Network.

The PARIS2 dataset expanded known eukaryotic snoRNA-tRNA interactions from two to more than 900, including nearly all nuclear-encoded tRNAs. For C/D snoRNA-target chimeras, fragments mapped to snoRNAs piled around the D/D′ guides, as expected. To test whether PARIS2-captured interactions are energetically favorable, we shifted tRNA-mapping fragments. The PARIS2 chimeras, but not randomly shuffled ones, produced a deep MFE valley at the target sites. Furthermore, snoRNA-tRNA chimeras often extend beyond mature tRNA transcripts, suggesting that the interactions occur on pre-tRNAs prior to its folding into stable 3D structures and processing. The precise order of snoRNA-guided modifications and processing events, such as removal of leader and trailer sequences, remains to be determined. We noticed that a subset of snoRNAs bind both tRNAs and rRNAs, suggesting co-regulation of these two components in the translation machinery. For example, the newly discovered rRNA-targeting D101 also binds multiple tRNAs, primarily Pro and Glu tRNAs, using the same conserved D′ guide. Despite the lower crosslinking efficiency, we discovered several interactions between H/ACA snoRNAs and tRNAs that may guide Ψ, including the highly conserved TPC motif. MFE-based prediction of individual snoRNA-tRNA interactions and comparison with chimeric reads from PARIS2 further confirmed the validity of a large number of them, revealing specific modification hotspots in various tRNAs. In particular, several snoRNAs encoded in the introns of Rpl13a, i.e., U32A, U33, U34 and U35A, were identified as mediators of cellular stress. Our PARIS2 analysis revealed several tRNAs, especially for Gly and Val, are major targets of these snoRNAs, in addition to rRNAs.

CLIP Confirms snoRNP Interactions with tRNAs.

To validate the global snoRNA-tRNA network, we analyzed PAR-CLIP and eCLIP data for human snoRNP proteins (Van Nostrand et al. Nat Methods 13, 508-514 (2016); Gumienny et al., Nucleic Acids Res 45, 2341-2353 (2017)). Earlier studies failed to discover snoRNP-tRNA interactions due to lack of normalization. Using proper normalization and false positive controls, we discovered that human FBL, NOP56, and NOP58 bind between 60% and 93% of cyto-tRNAs, while human DKC1 binds 25-99% cyto-tRNAs, in addition to the known rRNA and snRNA targets. CLIP occasionally produces hybrid reads from interacting RNAs (Lu et al. Nat Commun 11, 6163 (2020); Kudla et al., Proc Natl Acad Sci USA 108, 10010-10015 (2011)). Re-analysis of CLIP data using CRSSANT (Zhang et al. Genome Res 32, 968-985 (2022)) revealed a few snoRNA-tRNA chimeras, most of which are consistent with PARIS2 data (n=954 interactions supported by either PARIS2 or CLIP). Similarly, yeast CLIP of FBL/NOP1, NOP56 and NOP58 enriched between 59 and 85% of cyto-tRNAs. Together, PARIS2, structure modeling, and CLIP analysis revealed nuclear tRNAs as a major group of targets of snoRNAs.

FBL is a Master Regulator of tRNA Modification.

To determine the functions of FBL and DKC1, we performed mass spectrometry on purified tRNA and 18S/28S rRNAs. Nm levels were reduced in both 18S/28S rRNAs and tRNAs upon FBL KD, while DKC1 KD did not change tRNA Nm levels except Cm. Both FBL and DKC1 are essential for cell survival, therefore, absolute measurement of FBL and DKC1-dependent Nm and P sites are impossible in the partial knockdown cell lines. Interestingly, we observed larger reductions in Nm levels in tRNAs than in rRNAs after FBL KD, suggesting stronger dynamic regulation of tRNA Nm levels. P level was reduced in rRNAs but not tRNAs after DKC1 KD, suggesting either interactions that do not guide tRNA modifications, or only few tRNA P sites are catalyzed by DKC1. Surprisingly Ψ was reduced after FBL KD in tRNAs, even though FBL does not have Ψ synthase activity, indicating cross regulation among RNA modifications. The reduction of Cm upon DKC1 KD and reduction of Ψ upon FBL KD are unexpected and could be due to several indirect mechanisms. C/D and H/ACA snoRNAs may bind and guide modifications on each other. Therefore, loss of C/D snoRNP activity may compromise H/ACA snoRNP functions, and vice versa. Alternatively, some of the modifications on tRNAs may be necessary for other modifications (e.g., Nm on tRNAs may be required for Ψ modification). Furthermore, Nm and Ψ can also be installed by stand-alone protein enzymes independent of snoRNAs, therefore, some of the modification reductions may be due to secondary defects of other tRNA modification enzymes.

To discover Nm sites in tRNAs, we applied dRMS to control and siFBL HEK293 cells. Reduced Nm levels upon FBL KD were observed in 149 sites, among which, PARIS2 captured guide snoRNAs for 15 sites. KD of EZH2, a known oncogene required for snoRNP assembly, also reduced tRNA modifications, suggesting a connection of snoRNA-tRNA interactions to cancer. Further quantification showed that multiple other tRNA modifications, such as m¹A, m³C, m⁵C, and dihydrouridine (D), were also reduced in tRNAs upon FBL KD, but not DKC1 KD, indicating that snoRNA-guided Nm sites are needed for some of the other modifications.

To determine whether yeast tRNAs are modified by snoRNPs, we re-analyzed published RMS data in three yeast mutants. Bcd1 encodes an essential factor in snoRNP assembly. The bcd1-D72A mutation causes cells to have low steady-state levels of box C/D snoRNAs, resulting in significant loss of Nm levels. The Dbp3 RNA helicase participate in snoRNA processing and recycling. The Dbp7 RNA helicase is required for snoRNA-dependent ribosome assembly. Loss of Bcd1 resulted in greater reduction of Nm levels in rRNAs than KD of Fbl/Nop1 and the Dbp3 or Dbp7 KO yeast. Analysis of RMS data in bcd1-D72A and Dbp3 KO yeast revealed several sites in tRNAs with reduced Nm (n=26747 nucleotide positions with calculated Nm levels).

FBL is Required for Global tRNA Stability.

To determine whether snoRNPs affect tRNA stability, we knocked down FBL and DKC1 in HEK293 and A549 cells using siRNAs. FBL, but not DKC1, KD increased fragments in the 15-50 nt range, either in the absence or presence of oxidative stress (arsenite). The fragments include tRNA halves (˜34 and 40 nts) and shorter ones below 20 nts from D and T loop cleavage. Stable shRNA KD of FBL and DKC1 in HEK293, A549 and HepG2 cell lines, and exposure to various stresses, such as arsenite oxidative stress, alkaline pH 9.0, and heat shock confirmed the siRNA KD results, demonstrating general role of FBL in tRNA stability.

To determine whether the increased fragmentation was due to intrinsic defects on tRNAs, we purified tRNAs from wildtype and siFBL cells, and incubated them with the purified endonuclease angiogenin (ANG). tRNAs from FBL KD cells are more susceptible to cleavage, generating a wide range of sizes, most of which are tRNA halves. The melting temperatures of purified total tRNAs were not changed after FBL KD, indicating that tRNAs were mostly folded properly. RNA-seq of fragments in the 15-50 nt range revealed increased global levels of cytosolic tRFs in siFBL, compared to siCtrl and siDKC1 cells. Together, these studies showed that FBL and the C/D snoRNPs act as a master regulator of global tRNA modification and stability, consistent with their early binding to pre-tRNAs.

D97/D133 snoRNAs Target an Extensive Set of tRNAs

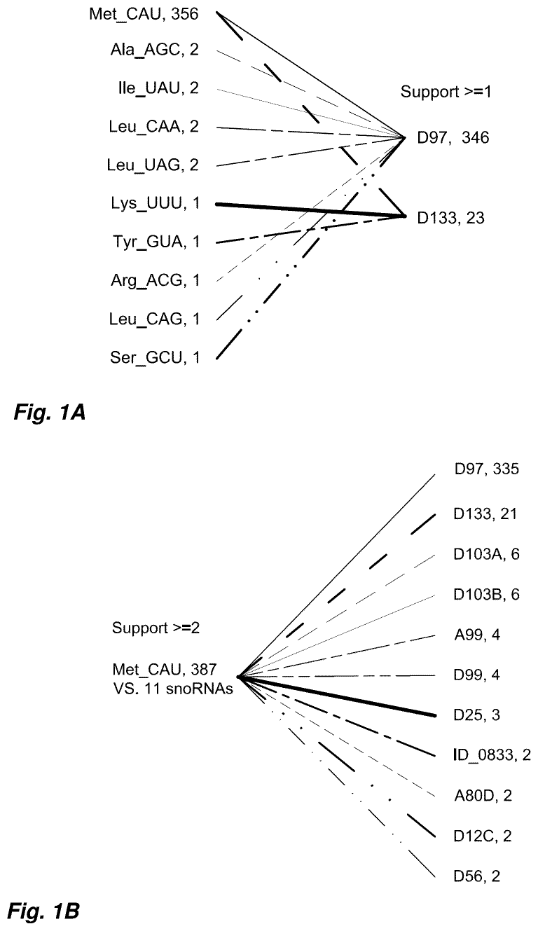

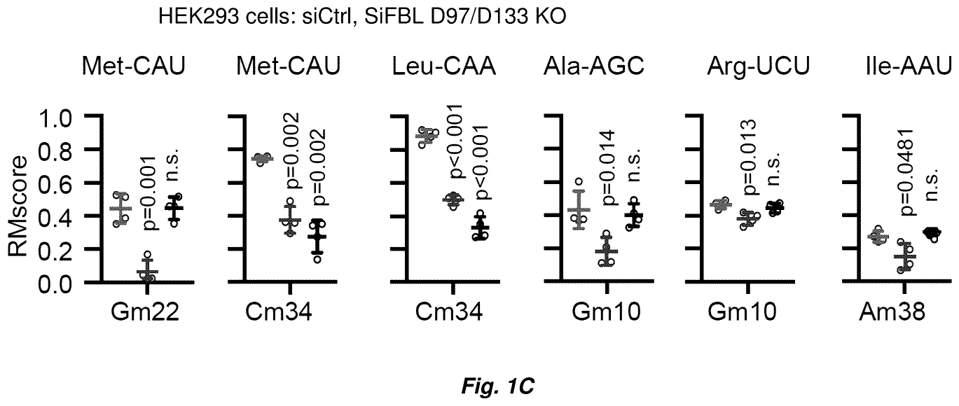

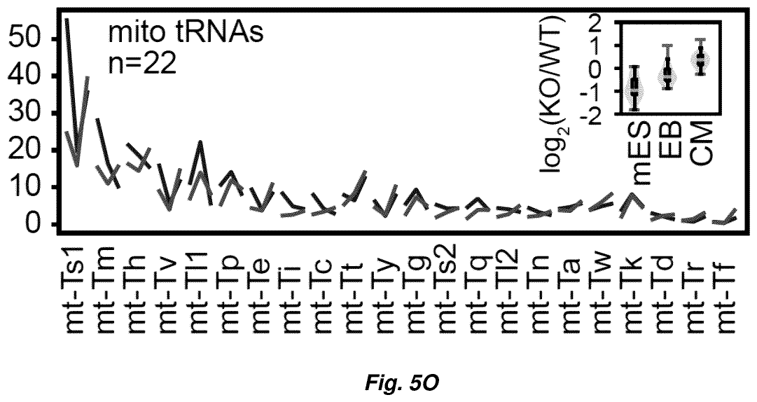

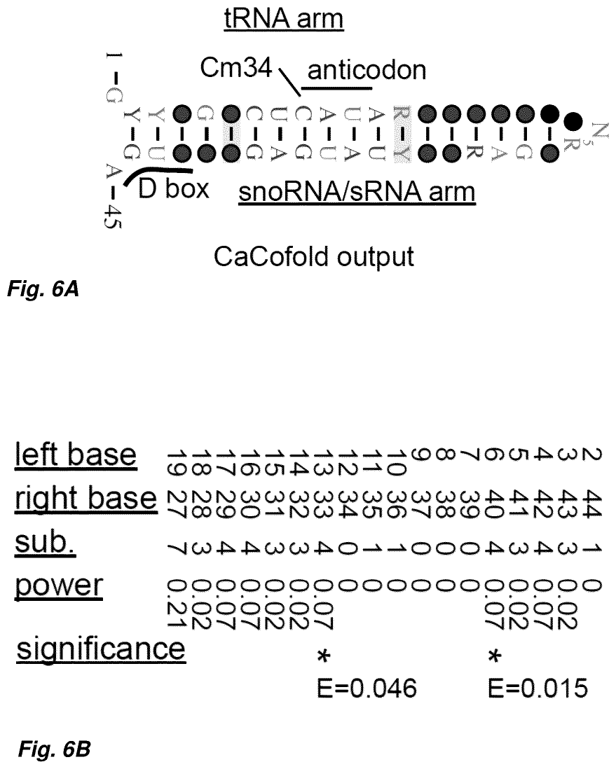

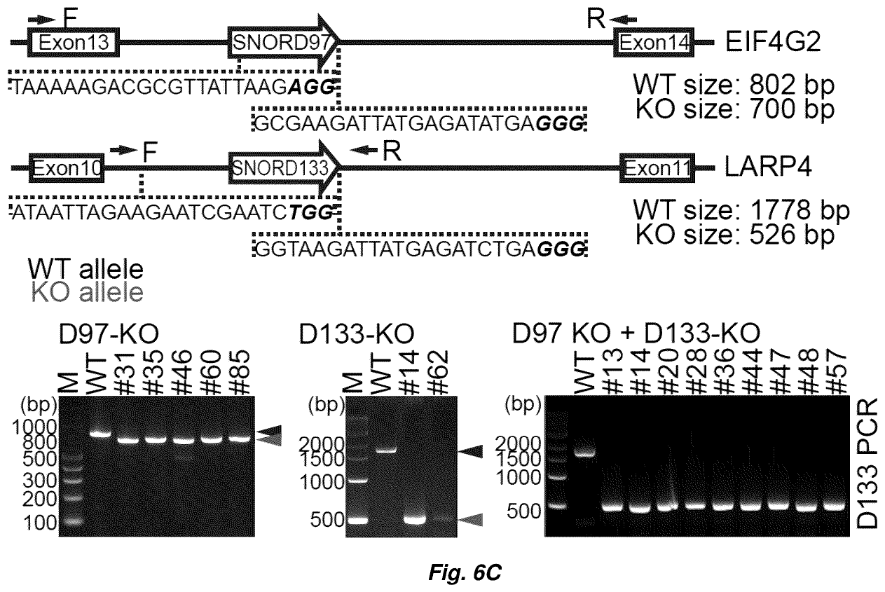

The conserved snoRNAs D97/D133, eMet-CAU tRNA, and their partners, form the strongest snoRNA-tRNA sub-network (FIG. 1A, FIG. 6A-B). Clustering resolved two duplex groups (DGs) connecting the D′/D guides to two distinct regions on eMet-CAU. These DGs form strong duplexes, predicting Nm sites at Gm22 and Cm34. PARIS2 also revealed Leu-CAA-5-1 as a new target for D97/D133, likely due to its close homology to eMet-CAU (FIG. 6C).

Exhaustive search revealed D97 homologs in archaea, some of which were previously predicted to guide archaeal Met tRNA modification at C34. Alignments of tRNAs and guides in human, plant A. thaliana, and 4 archaeal species revealed a conserved duplex of at least 11 base pairs (FIG. 6D). Interestingly, the target eMet-CAU tRNAs in plants and archaea have introns in the anticodon loop and participate in the extended duplex, further supporting that pre-tRNAs are snoRNA targets, and splicing likely occurs after the snoRNA-guided modifications. R-scape and CaCofold revealed two significantly covaried base pairs, in addition to 4 invariable base pairs, confirming deep functional homology among archaeal and eukaryotic guide RNAs for eMet Cm34 (FIGS. 6E-F). This analysis also revealed a conserved function for the poorly studied tRNA introns in guiding tRNA modifications.

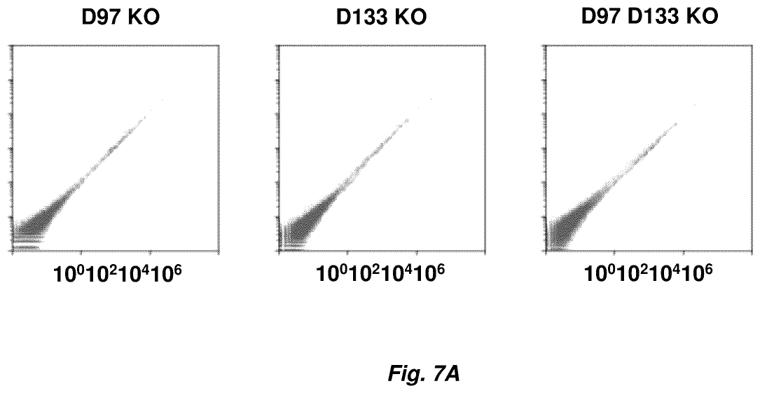

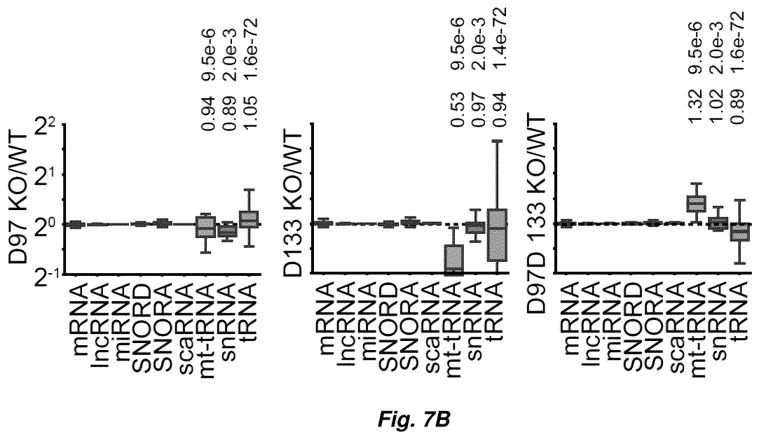

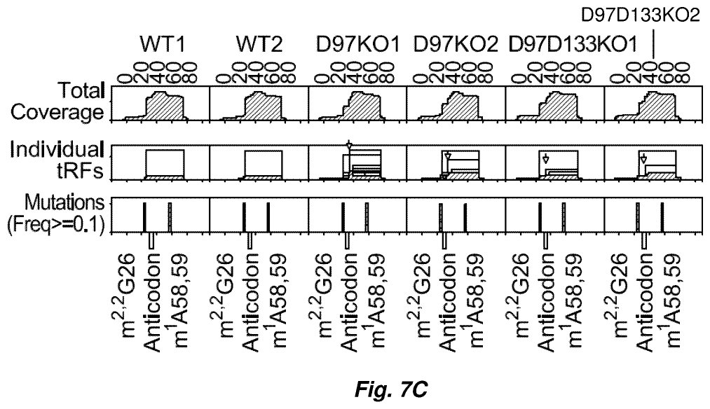

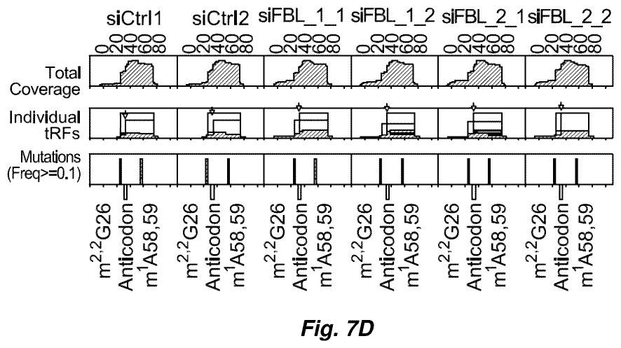

Other D97-tRNA interactions are also supported by strong duplexes despite the lower numbers of chimeric reads. The Nm levels at 6 sites in 5 tRNA targets are reduced either in FBL KD or D97/D133 double KO, or both, confirming the extended interaction network (FIG. 1B, FIG. 6G). The differential effects of FBL KD and D97/D133 snoRNA KO on Nm levels suggest additional snoRNA guides for these sites (FIG. 1B, n=44540 for all predicted snoRNA-tRNA interactions). The sub-stoichiometric and variable Nm levels suggest heterogeneity in the tRNA population, and potential alternative functions in the differentially modified tRNA molecules. We further used computational prediction to validate the subnetwork captured by PARIS2. Predicted D97 targets on all 432 human cyto-tRNA genes were aligned to a standard tRNA, revealing many potential sites, and three hotspots (10, 22 and 34), a subset of which were captured by PARIS2 and/or validated by dRMS, such as Gm22 and Cm34 in Leu and eMet tRNAs, Arg-UCU Gm10, and Ile-AAU Am38. Prediction of D133 targets revealed primarily interactions with the D guide due to divergence of sequence of the D′ guide (FIG. 6H). RNA-seq of RNA fragments in the 15-50 nt range confirmed the elevated levels of tRNA halves after D97/D133 KO (FIG. 7A-D). Together, the integration of PARIS2 and optimized dRMS revealed an extensive and deeply conserved D97/D133-tRNA sub-network.

D97/D133 snoRNAs Balance the tRNA Pool for Efficient Translation.

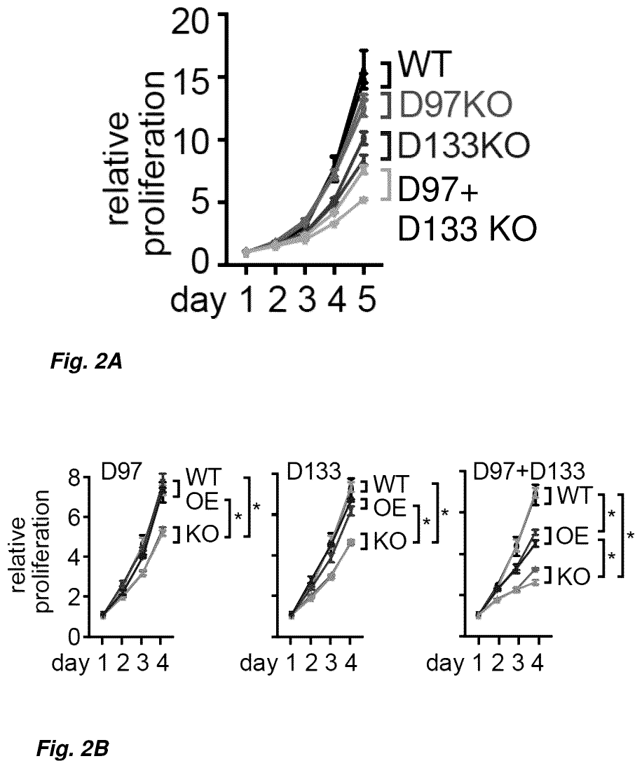

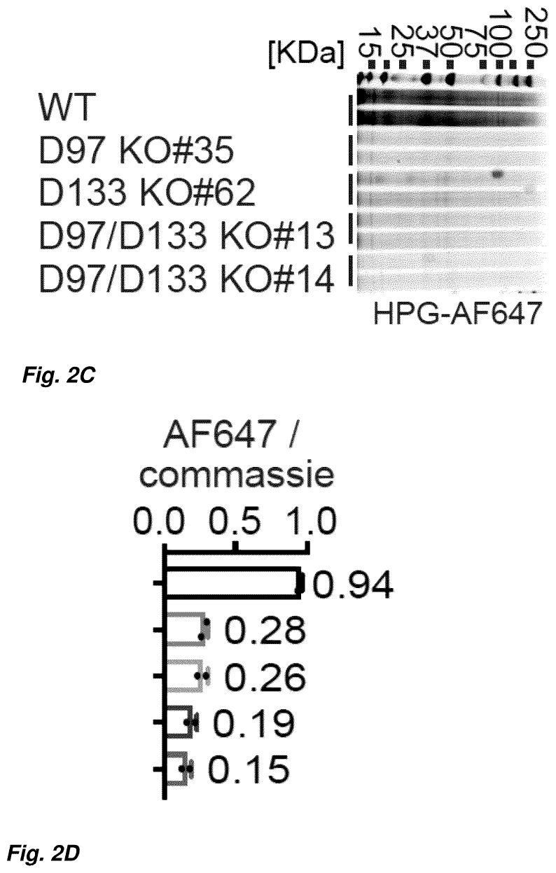

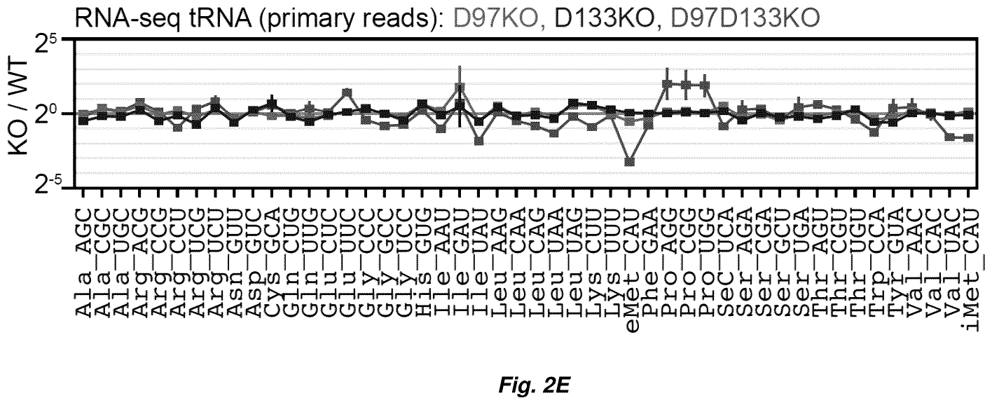

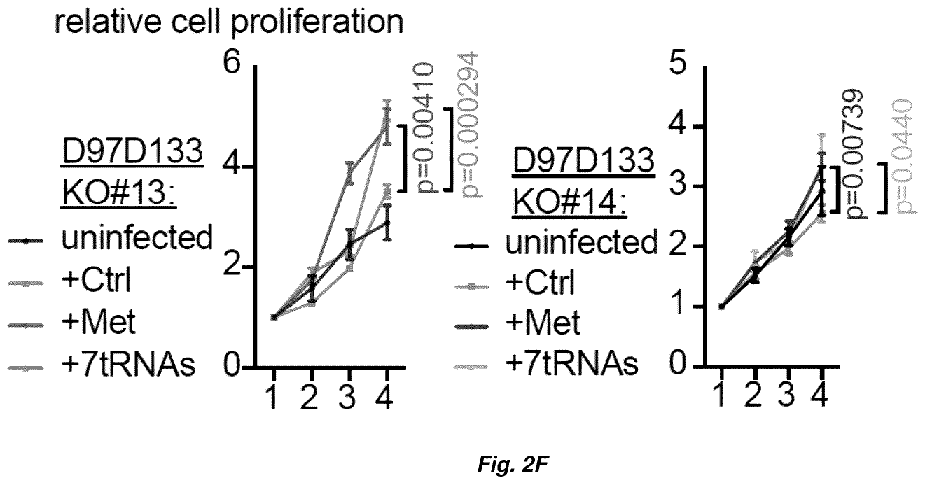

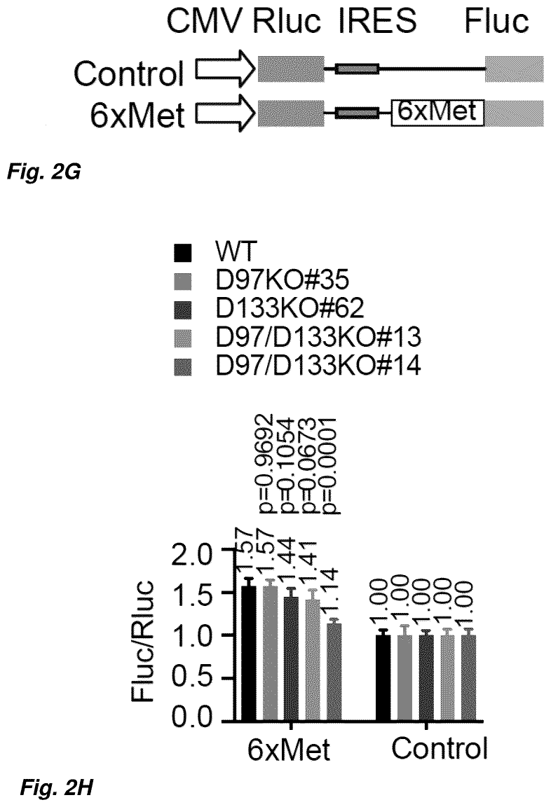

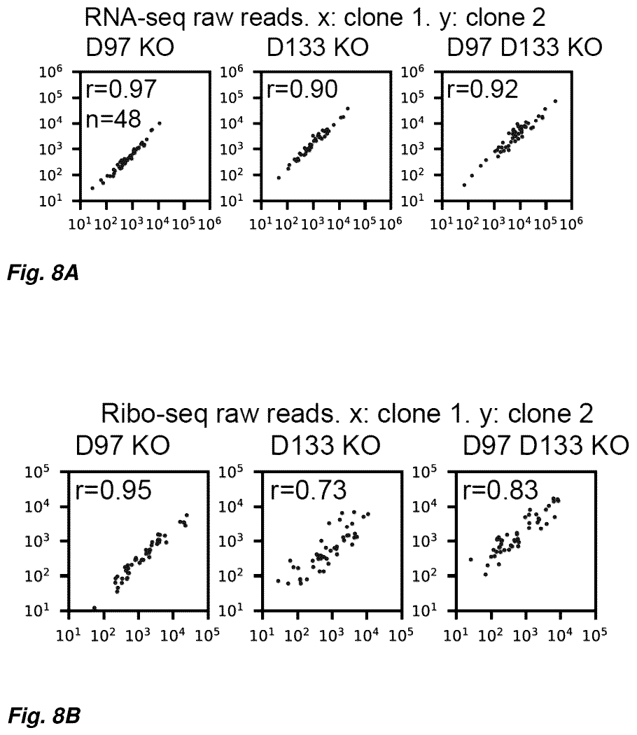

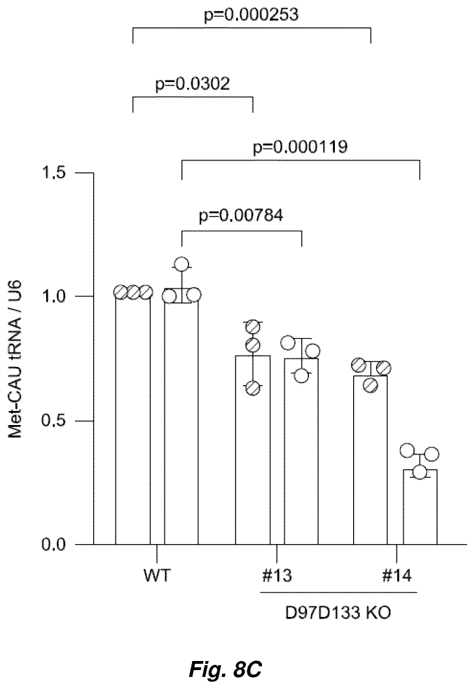

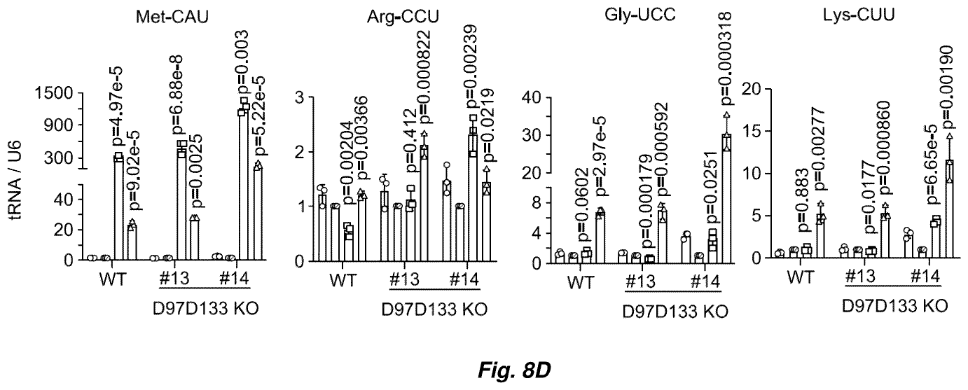

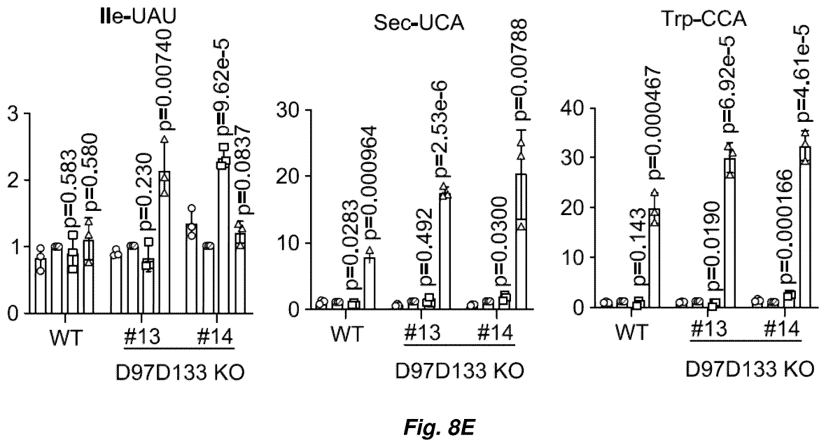

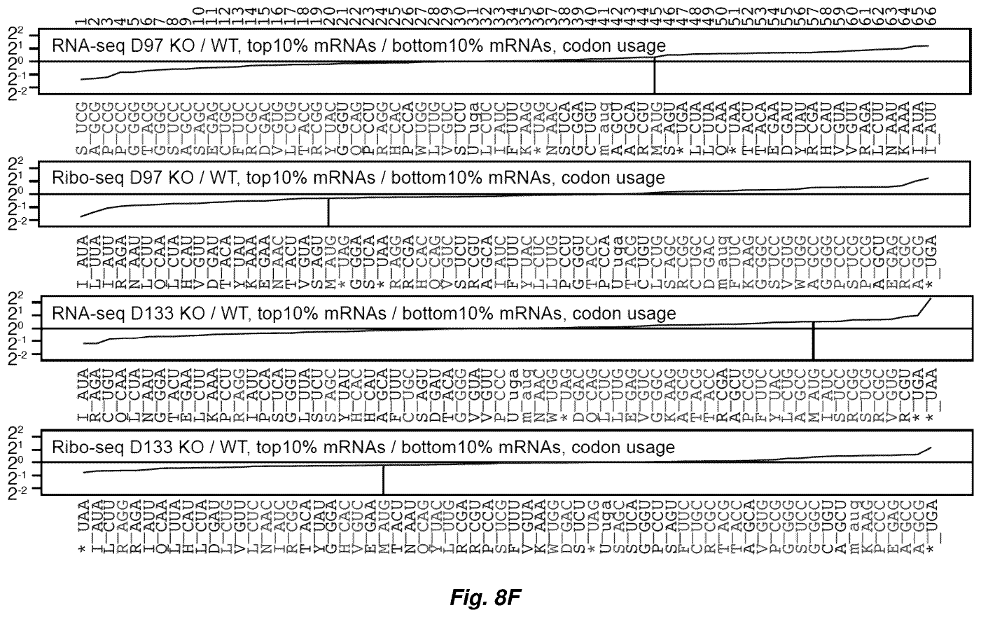

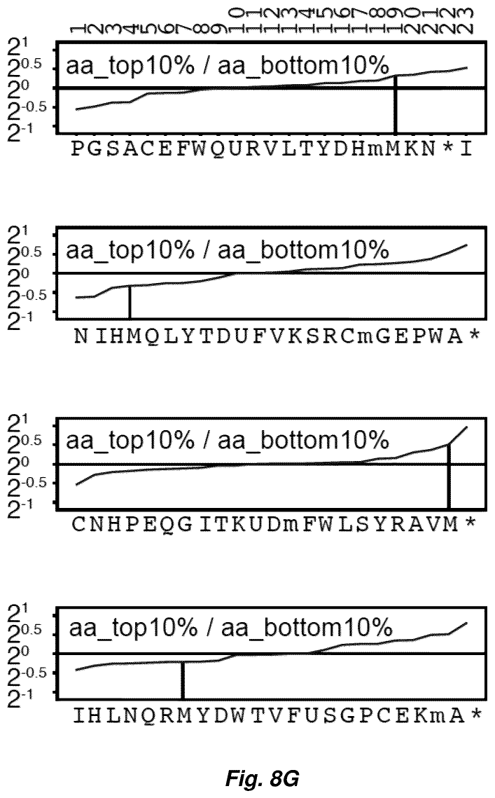

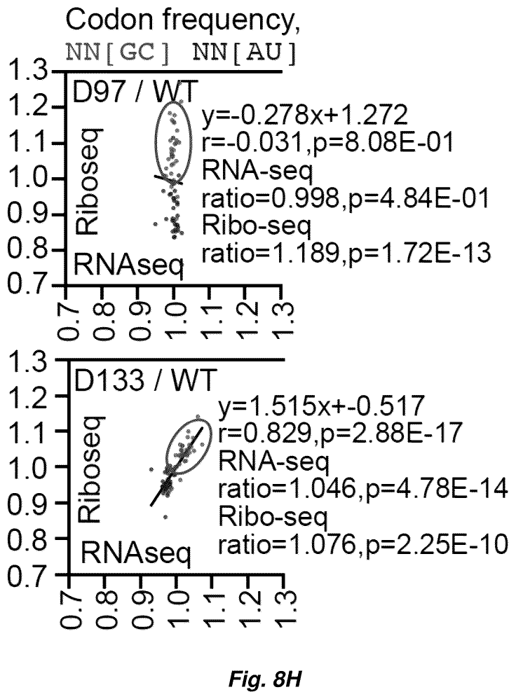

The extensive set of D97/D133 targets and the deeply conserved interactions with Met/Leu tRNAs across archaea and eukaryotes suggest this network is essential, however, little is known about their functions. CRISPR KO of either or both D97/D133 in HEK293 cells significantly reduced cell growth (FIG. 2A). snoRNA overexpression rescued the defects, confirming that the snoRNAs are essential (FIG. 2B). Labeling of nascent peptides by the Met analog HPG revealed dramatically reduced global translation in all KO strains (FIG. 2C-D). To determine the cause of the defects, we performed RNA-seq and ribosome profiling (ribo-seq) Ingolia et al., Science 324, 218-223 (2009). (FIG. 8A-B). Single KO strains did not change tRNA levels, even though each snoRNA paralog is necessary for tRNA modifications, however, the double KO reduced expression of several tRNAs, including eMet, Leu, Ile, etc. (FIG. 2E), many of which are targets of D97/D133 (FIGS. 1A-C). On the other hand, Pro tRNAs, which are not D97/D133 targets, were significantly upregulated, suggesting secondary effects of the KOs on the tRNA pool.

To confirm that reduced tRNA activity and levels are responsible for the translation defects, we infected HEK293 cells with lentiviruses expressing no insert (control) or tRNA eMet-CAT, or a mixture of 7 lentiviruses expressing eMet-CAT, Arg-CCT, Gly-TCC, Lys-CTT, Ile-TAT, Sec-TCA, and Trp-CCA (7tRNAs), which are reduced in the D97/D133 double KO. After puromycin selection, these tRNAs are expressed at high levels (FIG. 8C-D). Overexpression of both eMet-CAT and the 7-tRNA mixture partially rescued growth defects in KO lines (FIG. 2F). To test whether the Met-AUG codon bias is responsible for the reduced translation in KO cell lines, we further constructed reporters (FIG. 2G). The insertion of 6×Met-ATG decreased protein synthesis in KO cell lines relative to WT (FIG. 2H), confirming that the D97/133 KO caused eMet-CAU tRNA defects. Together, these studies demonstrate, for the first time, an essential role of snoRNA-guided modifications in maintaining a balanced tRNA pool for efficient translation.

### D97/D133 Govern Met-Biased Gene Expression Programs.

Met is one of the few reversibly oxidizable amino acids in proteins. Dedicated enzymes in all three domains of life, such as methionine sulfoxide reductases, repair oxidized Met to protect proteins with long half-lives or close to ROS sources. In particular, spliceosomal proteins, nucleic acid binding proteins, and mitochondrial proteins encoded by both the nuclear and mitochondrial genomes have some of the highest ratios of Met residues in the proteome in higher animals. On the other hand, the short-lived proteins involved in development, differentiation, and morphogenesis, are relatively depleted of Met. Therefore, the D97/D133 targeting of tRNAs, particularly Met, is likely a new mechanism to regulate Met codon and amino acid usage, and the corresponding gene expression programs.

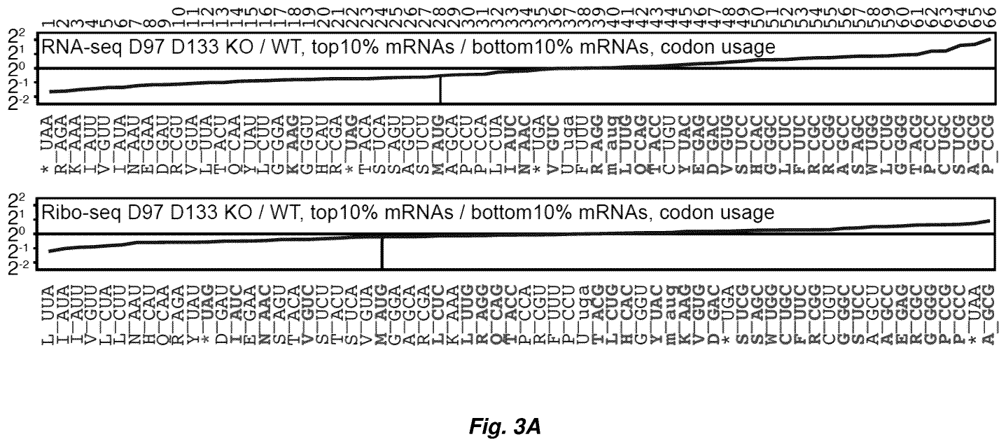

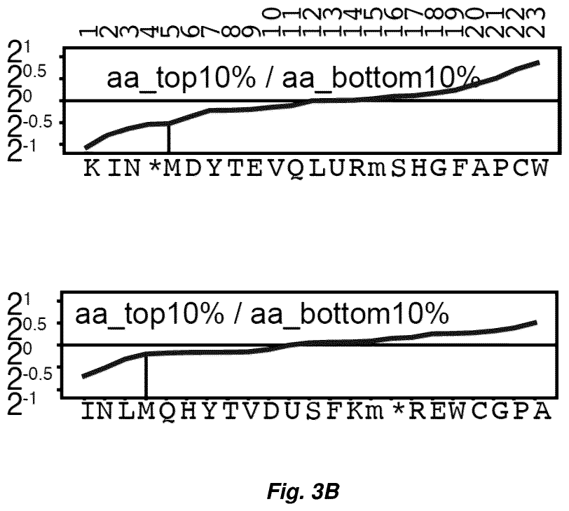

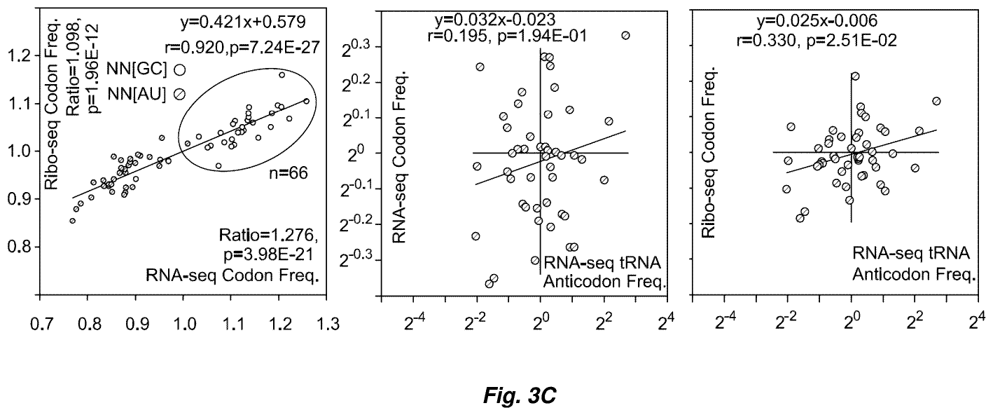

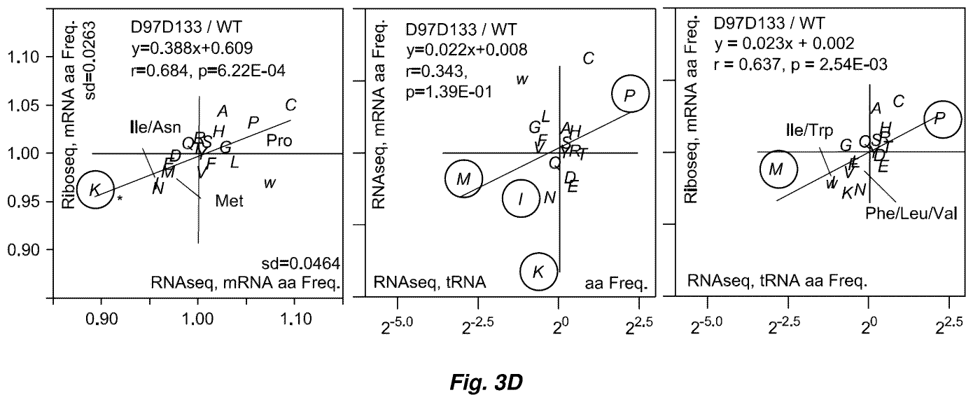

To test this hypothesis, we measured codon usage in both the transcriptome and translatome (ribosome-associated mRNAs). The usage of many codons, including Met-AUG, changed significantly in the D97/D133 double KO, and less so in the single KO strains (FIG. 3A). There is a clear enrichment of GC-ending codons and depletion of AU-ending ones, which correlate with stem cell self-renewal, differentiation, and multicellular functions. Usage of the Met amino acid is the fourth most reduced in the double KO in both the transcriptome and translatome (FIG. 3B). Strong positive correlations were observed between the transcriptome and translatome in the codon and amino acid usage changes in double KO vs. WT (FIG. 3C-D). The D97 and D133 single KO did not show the same trend of codon and amino acid frequency changes on the transcriptome, but changed codon and amino acid frequency on the translatome level, suggesting dose-dependent tRNA defects in the single vs. double KO cells (FIG. 8F-H).

Gene ontology (GO) analysis (Subramanian et al., Proc Natl Acad Sci USA 102, 15545-15550 (2005); Gu et al., Genomics Proteomics Bioinformatics (2022) doi.org/10.1016/j.gpb.2022.04.008) of KO cell lines revealed significant downregulation of genes with basic cellular functions, such as ribosome, spliceosome, mRNA metabolism, and oxidative phosphorylation in both the double and single KO cell lines (FIG. 8A), consistent with the reduced growth and translation (FIG. 2). Upregulated GO terms on both the transcriptome and translatome levels include differentiation, development, and morphogenesis, among others.

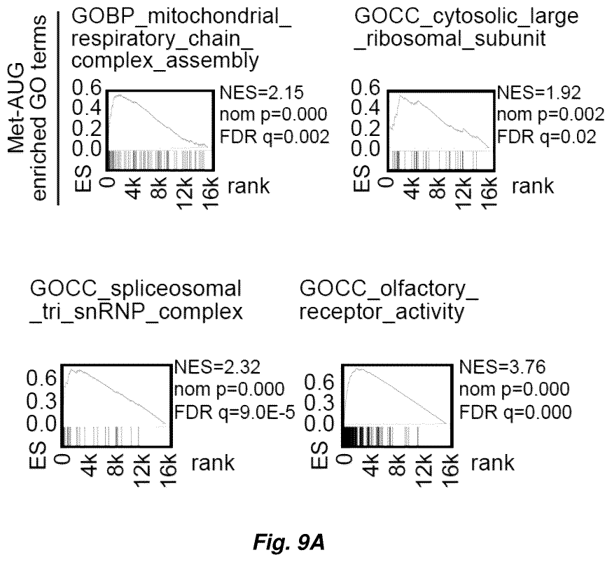

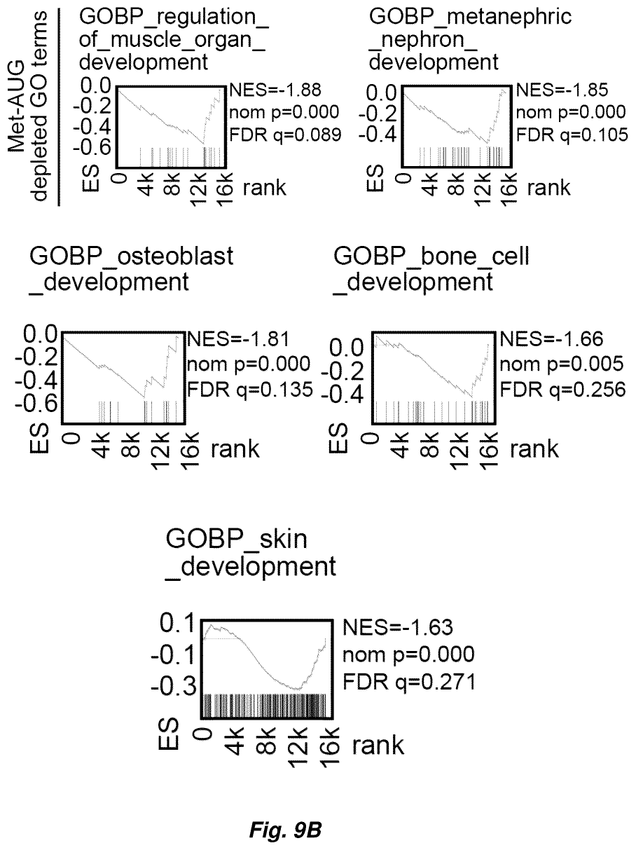

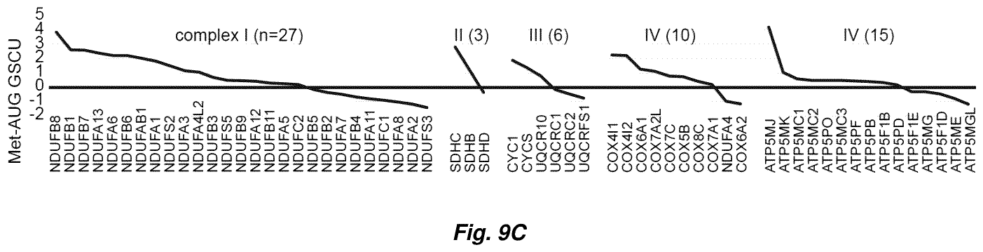

Interestingly, Met is depleted in the up-regulated GO terms on the transcriptome and translatome levels, and vice versa (FIG. 9). GO analysis of translation efficiency did not reveal similar terms, suggesting that the gene expression alteration for these GO terms are primarily on the transcriptome levels, likely due to changes in transcription and/or stability.

Both nuclear and mitochondrially encoded mitochondrial proteins are enriched in Met, suggesting convergent evolution to cope with oxidative stress. However, only the nuclear-encoded tRNAs are targeted by snoRNAs. The reduction of mitochondrial translation as measured by ribo-seq (FIG. 9A-C) is likely a secondary effect of cellular adaptation to coordinate translation in the two subcellular compartments. Nascent translation of mitochondrial genome encoded peptides, measured after cycloheximide inhibition of cytoplasmic translation, was all reduced in both the single and double KO cells, confirming the RNA-seq and ribo-seq measurements. Together, these studies revealed a critical role of Met codon usage in controlling the dichotomous gene expression programs.

Transcriptome and Translatome Codon Usage Depends on snoRNA Dose.

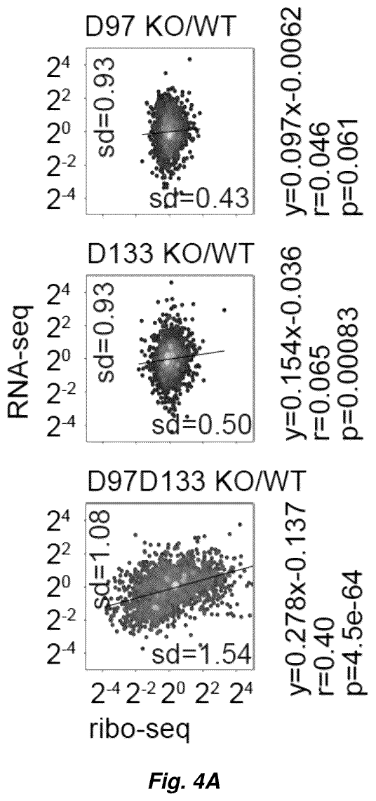

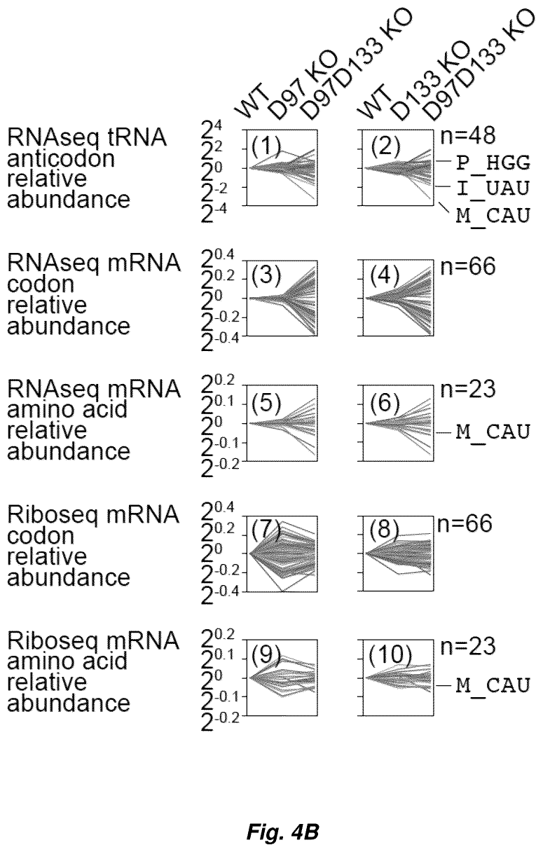

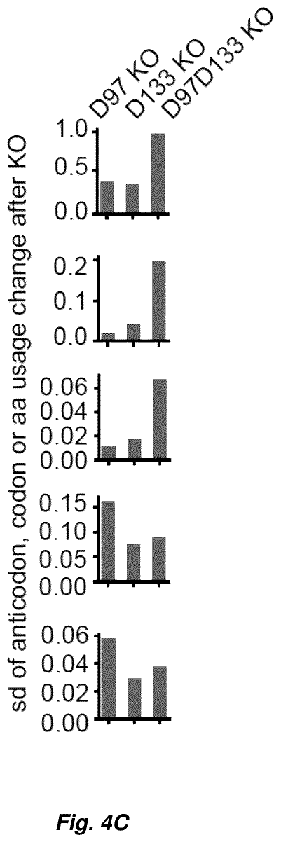

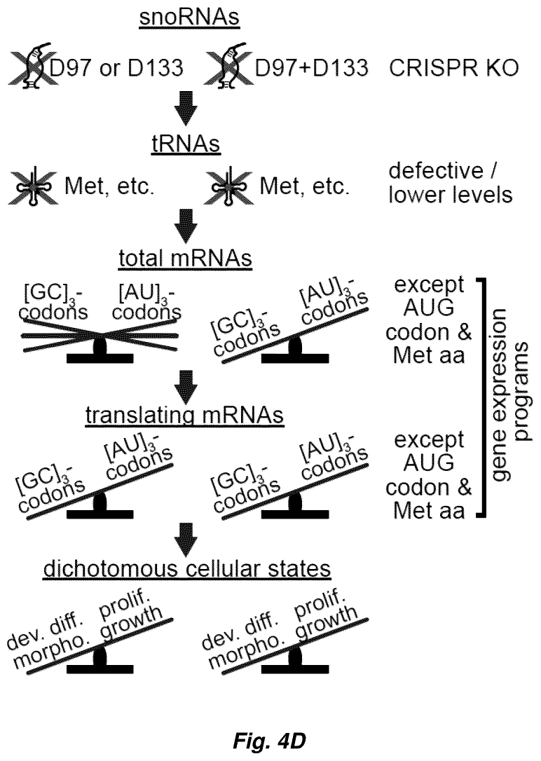

As shown above, the D97/D133 single and double KO cell lines reprogram the transcriptome and translatome to different extents (FIG. 3A-B vs. FIG. 8D-F). To further quantify the gene expression reprogramming, we measured standard deviations of expression fold changes in KO vs. WT. Single KO primarily affected translation (FIG. 4A, top and middle, higher variation on the translatome level), while double KO affected both the transcriptome and translatome (FIG. 4B, bottom, higher variation on the transcriptome level). Double KO induced bigger changes in relative tRNA levels (FIG. 4B). On the transcriptome level, double KO induced larger differences in codon and amino acid usage, than single KOs. On the translatome level, double KO induced similar changes in codon and amino acid usage as single KOs (FIG. 4B).

Comparing variations between RNA-seq and Ribo-seq, the single KO exerted more effects on the translational level, whereas the double KO already showed large differences on the transcriptome level, which persisted in the translatome level (FIG. 4B, panels 3 vs. 7, 4 vs. 8, 5 vs. 9, and 6 vs. 10, and FIG. 4C).

Together, the RNA-seq and ribo-seq in D97/D133 single and double KO HEK293 cells suggest a global reprogramming of the transcriptome and translatome (FIG. 4D). Loss of the snoRNAs resulted in defective target tRNAs, especially eMet-CAU, accompanied by reduced usage of Met and other codons in the transcriptome and translatome levels, leading to an imbalance in two competing gene expression programs: proliferation vs. differentiation/development/morphogenesis. The tipped balance between GC- and AU-ending codons, which has been observed in other biological contexts of cellular proliferation vs. development³⁻⁴, are likely induced by the defects in D97/D133 target tRNAs here in HEK293 cells. The double KO induced larger differences on the transcriptome level, suggesting adaptation of the cells to dramatically reduced levels of eMet-CAU and several other tRNAs (FIG. 4D, total mRNAs). Together this quantification revealed dose-dependent effects of D97/D133 on the transcriptome and translatome programs.

### Mouse D97/D133 Regulate Codon-Biased Stem Cell Differentiation.

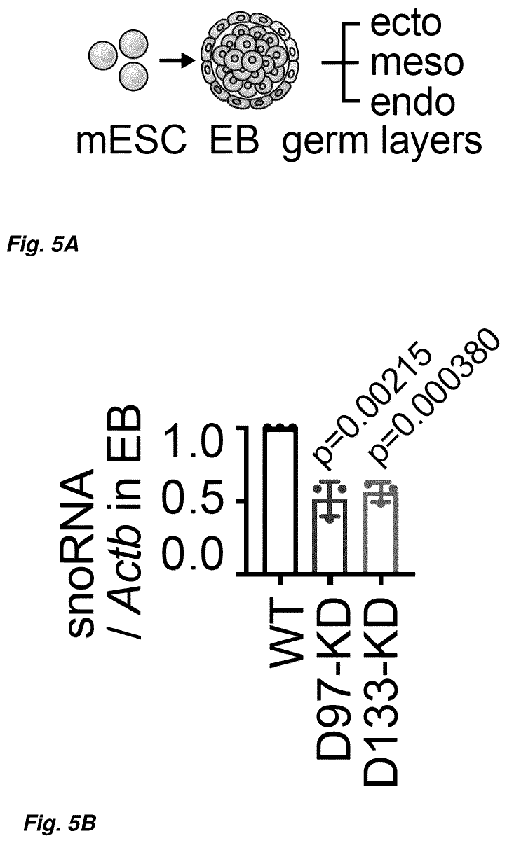

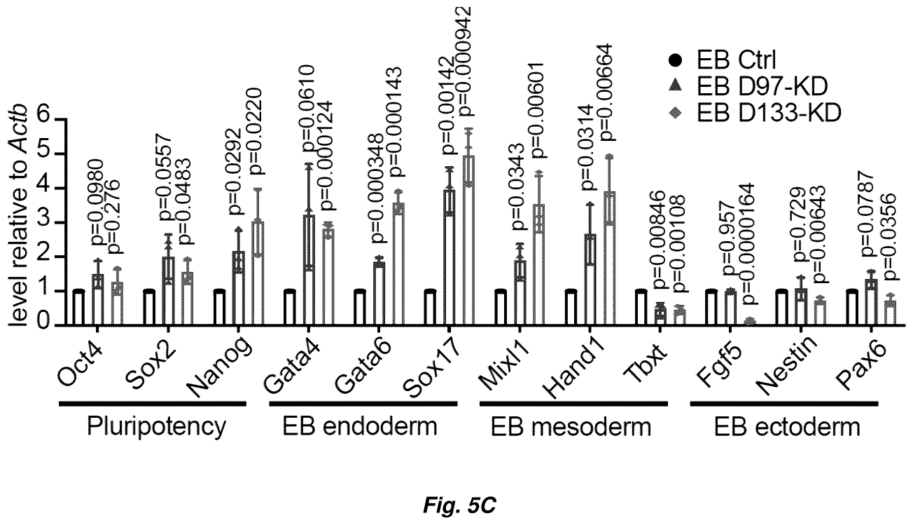

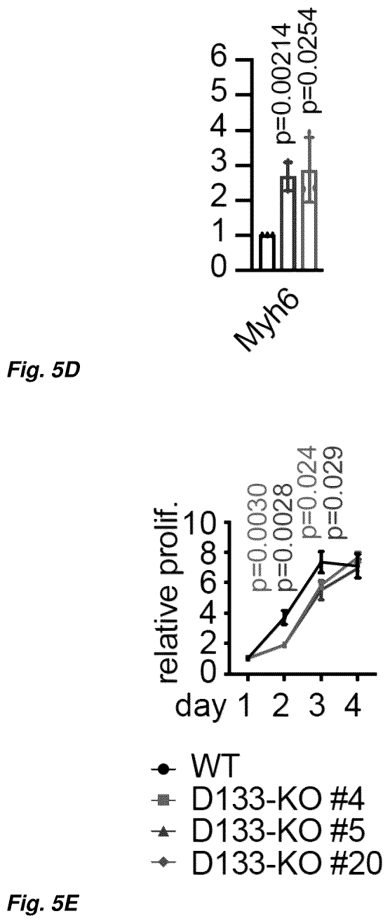

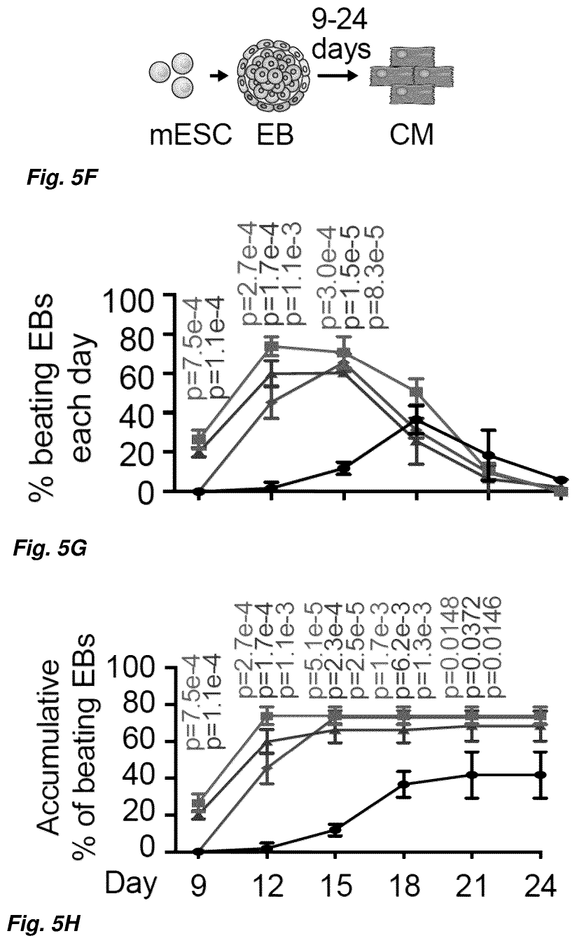

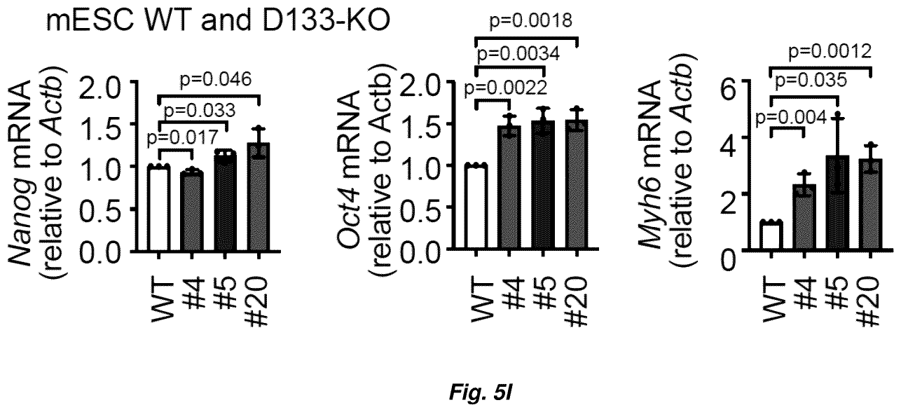

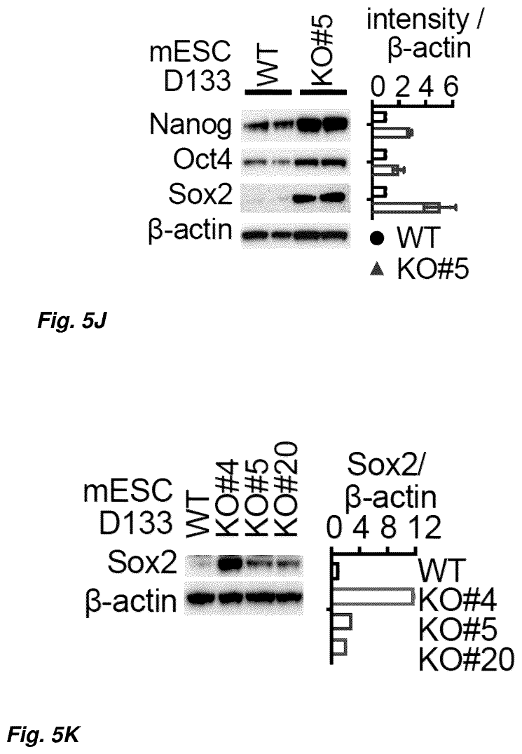

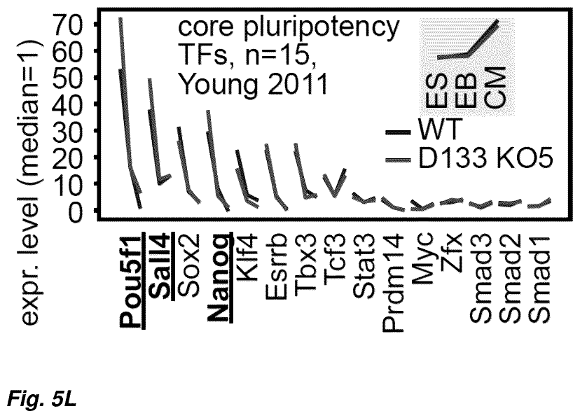

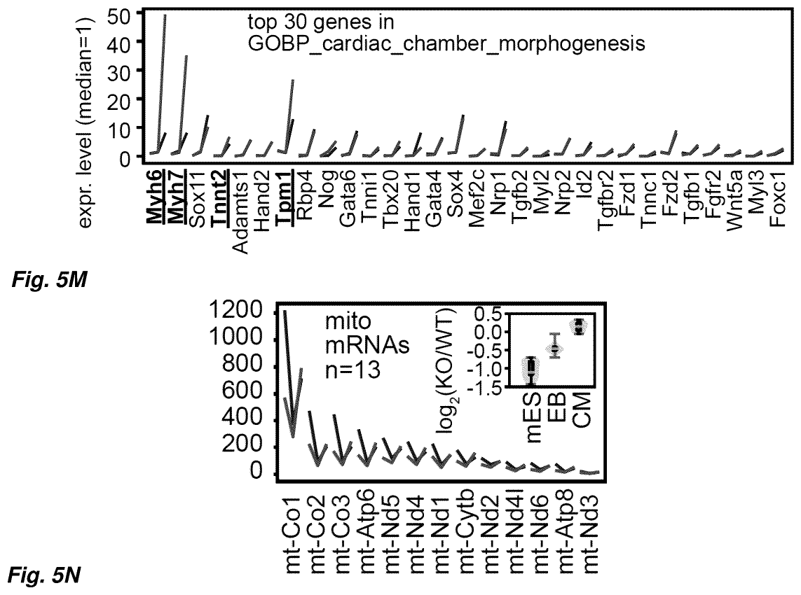

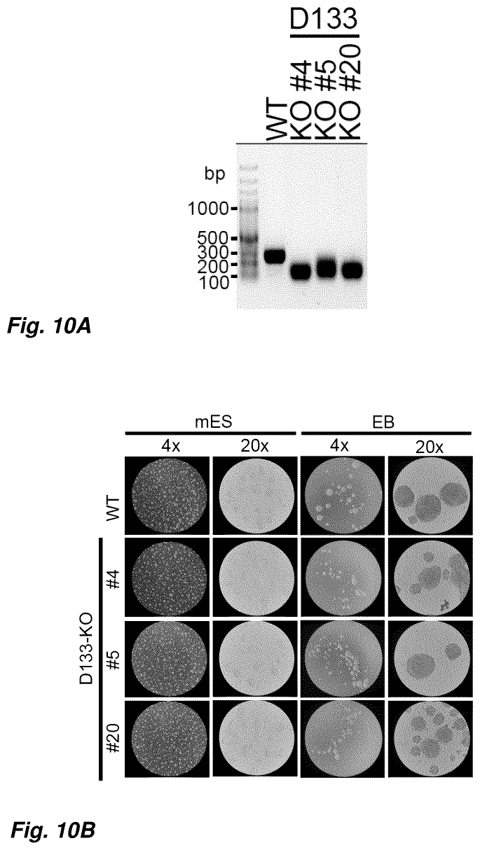

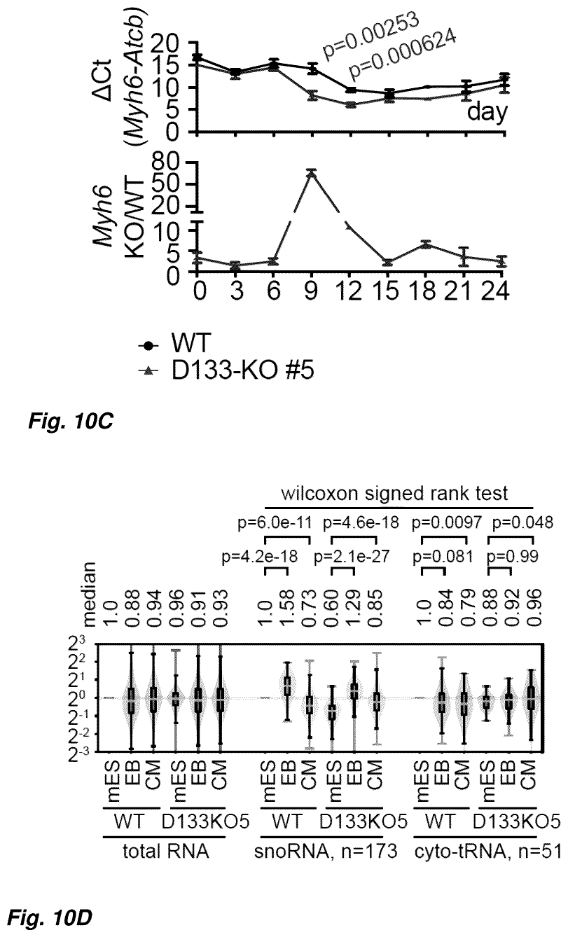

Given the codon-biased induction of development-related gene expression programs in human HEK293 cells after D97/D133 KO, we tested whether these snoRNAs regulate mES self-renewal and differentiation into embryoid bodies after ASO KD of mouse Snord97 and Snord133 (D97/D133) (FIG. 5A-B). Pluripotency-related mRNAs increased while markers for the three germ layers were skewed, favoring the mesoderm and endoderm (FIG. 5C). In particular, the cardiomyocyte (CM) Myh6 increased (FIG. 5D). These results are consistent with the upregulation of genes involved in differentiation, development, and morphogenesis in HEK293 cells (FIG. 3). To further analyze the roles of D133 snoRNA in CM differentiation, we knocked it out using CRISPR (FIG. 10A). Cell growth slowed down significantly, similar to HEK293 cells (FIG. 5E). mES gross morphology and self-renewal remained the same (FIG. 10B), yet all KO clones significantly increased the speed and efficiency of CM formation, from ˜40% to ˜75% (FIG. 5F-H). The Myh6 mRNA was induced earlier and higher in the D133 KO throughout differentiation (FIG. 10C). At the same time, mRNA and protein levels of pluripotency markers increased, such as Nanog, Sox2 and Oct4, similar to the ASO KD (FIG. 5I-K). Together these phenotypes confirmed the codon-biased gene expression programs in D97/D133 KO HEK293 cells.

To determine the mechanisms driving the faster and skewed differentiation, we performed RNA-seq in mES, EB and CM. The mES D133 KO did not change relative tRNA levels between KO and WT (FIG. 10D-E), consistent with the HEK293 D97/D133 single KO lines. At each stage, the KO induced distinct differences in the transcriptome. Pluripotency TFs dropped from ES to EB and CM stages while cardiac genes were induced, confirming successful differentiation. Pluripotency factors Pou5f1 (Oct4), Sall4 and Nanog increased after KO in the ES stage (FIG. 5L), while cardiac development factors increased in D133 KO vs. WT (FIG. 5M), consistent with the qRT-PCR and western blots (FIG. 5I-K). Mitochondrial transcripts significantly reduced in the ES stage (FIG. 5N-O). The return to normal of mitochondrial gene expression in D133 KO CMs is surprising but consistent with the more efficient differentiation to CM.

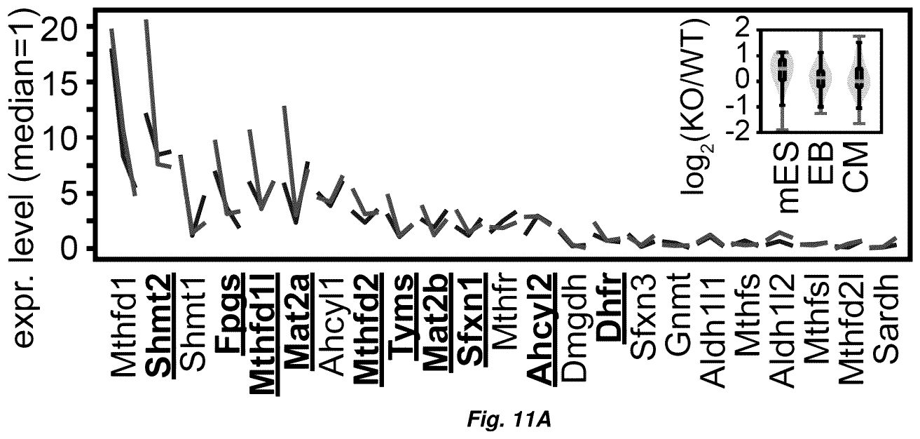

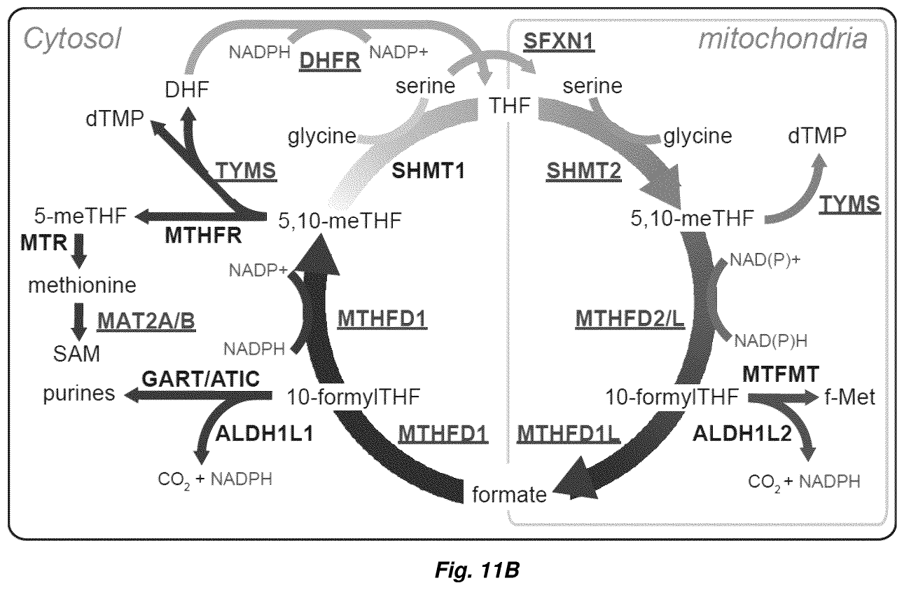

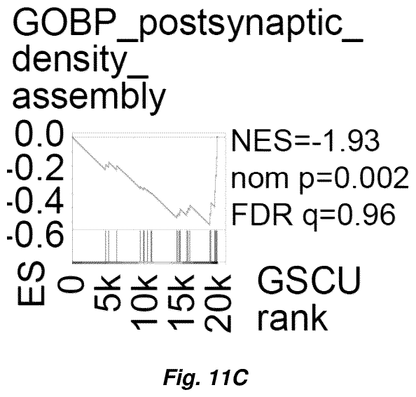

Interestingly, several other mitochondrial metabolic processes, such as the one-carbon cycle, are upregulated, suggesting dysregulation of metabolites with potential roles in altering epigenetic status of D133 KO stem cells (FIG. 11A-B). Gene set enrichment analysis revealed consistent upregulation of development-related terms, especially cardiac development (FIG. 11C). In contrast, mitochondrial electron transport, neurodevelopment related genes were down-regulated in KO vs. WT, again consistent with the D133 KD studies. This analysis revealed enhanced pluripotency and CM gene expression programs in mouse D133 KO, consistent with the skewed and more efficient CM differentiation phenotype. Together, the KD and KO studies in human and mouse cells demonstrated a critical role of the D97/D133 target tRNAs in controlling the dichotomous cellular states of proliferation vs. development (FIG. 4D).

This comprehensive study presents important discoveries and conceptual advances. We discover an extensive snoRNA targetome that include multiple classes of ncRNAs, solving a long-standing mystery of orphan snoRNAs. Integrated PARIS2 interactome capture, structure modeling, conservation analysis, normalized analysis of CLIP, and optimized dRMS modification mapping demonstrated a conserved global snoRNA-tRNA interaction network. The 2′-O-methylation of pre-tRNAs by FBL controls global tRNA modifications beyond 2′-O-methylation, and tRNA stability. Specifically, we discover a subnetwork of D97/D133-tRNA interactions that are required for a balanced tRNA pool, cellular proliferation, and translation (FIGS. 1-2). Loss of D97/D133 tipped the balance between the dichotomous programs of proliferation vs. development, as a result of the need for increased usage of the antioxidant Met in proliferation-related proteins (FIGS. 3-4). Consistently, in mouse ES cells, codon-biased gene expression promoted and skewed stem cell differentiation without compromising pluripotency (FIG. 5). Together, this study revealed a new class of regulators for codon biased gene expression programs and cellular states.

Despite extensive efforts in the past 3 decades to discover snoRNA targets, the vast majority of snoRNAs remain orphans. Technological advances in recent work (PARIS2) (Zhang et al. Nature Communications 12, 2344 (2021) and presented here (dRMS) are beginning to reveal complex modification networks with nucleotide resolution, and provide new mechanistic insights. While earlier studies suggested these interactions typically range between 10 and 21 bps, the interactions discovered here span a bigger range. The frequently detected bipartite duplexes for D/D′ guides likely strengthened the stability of some of the otherwise weaker interactions (e.g., FIG. 1A-C). However, we cannot exclude the possibility that a subset of them represent target scanning intermediates, or may function beyond guiding modifications (e.g., folding chaperone, guided processing, and RNA quality control), like the well-studied U3, U8 and U13. Several tRNA Nm sites are sub-stoichiometric, suggesting heterogeneous tRNAs where modification variants may have different functions (FIG. 1I).

The D97/D133 single and double KO reprogram the transcriptome and translatome via distinct mechanisms. In single KO strains, the reduced translation of Met-enriched proteins suggests that eMet tRNAs are defective, even though tRNA levels remain constant. The double KO significantly reduced several D97/D133 target tRNAs, especially eMet-CAU, reprogramming both the transcriptome and translatome to adapt to the skewed tRNA pool. Beyond the Met-AUG codon, the transcriptome of double KO cells exhibited a remarkable dichotomy of decreasing A/U ending codons, and increasing G/C ending ones, nominating the D97/D133-tRNA sub-network as a new regulator of the dichotomous programs rooted in wobble position bias of A/U and G/C content. Together, this work revealed a snoRNA-controlled cellular translation economy: specific snoRNAs regulate target tRNA activity and levels—the “supply”, which influences the corresponding codon usage in mRNAs—the “demand” (FIG. 4D).

It is not a coincidence that D97/D133 regulate the dichotomous programs of proliferation vs. development. The D97/D133 snoRNA-tRNA network is conserved in many species across eukaryotes and archaea (FIG. 6). The antioxidant role of protein-intrinsic Met is conserved across all three domains of life and its usage is highly enriched in proteins that have longer half-lives and/or need higher ROS-resistance, and relatively depleted from genes involved in development, differentiation, and morphogenesis (FIGS. 2-3). Therefore, the regulation of proliferation vs. development by D97/D133 represents an “evolutionary inevitability”. However, the precise mechanisms of the observed ES differentiation phenotypes remain poorly understood. In addition to the codon usage bias that directly alter levels of pluripotency and differentiation TFs, skewed mitochondrial metabolism is another possibility. The reduced respiration chain components and concurrent increases in other metabolic branches, such as one-carbon metabolism (FIG. 11), may alter key metabolites that participate in DNA, RNA and histone modifications to control stem cell epigenetics.

Since biased usage is common across all codons and amino acids, and underlies specific gene ontologies, the extensive snoRNA-tRNA network covering nearly all nuclear-encoded tRNAs suggests much broader impacts of snoRNAs in codon-biased gene expression programs in various biological and disease contexts. A large number of snoRNA-guided tRNA modifications may form a combinatorial regulatory code, like the histone modification code.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Material and Methods

PARIS2 library preparation. Briefly, cells were crosslinked with psoralen and 365 nm UV and collected to extract total RNA or chromatin-associated RNA (Zhang et al., Nature Communications 12, 2344 (2021)). snoRNAs and their crosslinked targets were enriched from the total RNAs using biotinylated antisense oligos. All RNA samples were fragmented and crosslinked fragments were enriched using the DD2D gel method. After proximity ligation, and reversal of crosslinks by 254 nm UV, the RNA samples were ligated to barcoded adapters, circularized, and amplified by PCR. The cDNA libraries were sequenced by NovaSeq 6000 (SE 100 bp).

CRISPR/Cas9-mediated snoRNA knockout. All snoRNA guide RNAs were designed by using Broad's CRISPick algorithm (Kim. et al. Nat Biotechnol 36, 239-241 (2018); DeWeirdt. et al. Nat Biotechnol 39, 94-104 (2021)). Guide RNAs used in CRISPR/Cas9 were cloned into lentiCRISPRv2 vector (Addgene, 52961). HEK293T cells were transfected with equal amounts of lentiCRISPRv2 vectors carrying two-guide RNAs flanking the snoRNA to be deleted, followed by clonal selection under puromycin and clone expansion.

Genotypic characterization was performed using PCR amplification. The PCR products of positive clones with homozygous deletion were validated by agarose gel and Sanger sequencing.

Lentiviral overexpression of snoRNAs and tRNAs. The sequence of human snoRNAs and tRNAs were respectively constructed into pLV-EF1a-IRES-Puro (Addgene, #85132) using BamHI and EcoRI cloning sites. Lentivirus was packaged by co-transfecting the above constructs with pVSVG and psPAX2 plasmids into HEK293T cells. The medium was changed 24 hours post-transfection. The lentivirus supernatants were collected at 48 hours and 72 hours and clarified with a 0.45 M filter. Lentivirus was either used directly for experiments or stored at −80° C.

Mouse embryonic stem cell (mES) differentiation. The CM differentiation procedure is based on a previous study with adjustment for the particular purpose of time course observation of the CM beating activity (Batista et al., Cell Stem Cell 15, 707-719 (2014).). Briefly, mES cells were plated at a density of 2×10⁵ cells/mL in ultra-low attachment plates in cardiomyocyte differentiation medium (CMD) (DMEM-High Glucose (CORNING)) supplemented with 15% Fetal Bovine Serum (Gibco), 1% Pen Strep (Gibco), 1× GlutaMax (Gibco), and 1 mM Ascorbic Acid (Sigma-Aldrich, A4544)) to induce embryoid body (EB) formation. On day 3, the medium was replaced with fresh CMD medium; on day 6, EBs were re-suspended in fresh CMD medium and replated on 0.2% gelatin-coated plates. During day 9 to day 24, the number of beating patches of cells was quantified in triplicate for each cell line and the CMD medium was changed every 3 days.

Data and code availability. The raw and processed total RNA seq, small RNA seq, optimized denatured RiboMeth-seq (dRMS), ribo-seq and PARIS2 data was deposited to Gene Expression Omnibus (GEO) with accession number GSE234689 (access code: kfgxyyyazpevhgh). Code is available at github.com/zhipenglu/snoRNA and github.com/minjiezhang-usc/snoRNAs_discovery.

Cell lines. HEK293 (ATCC, CRL-3216), A549 (ATCC, CCL-185) and SH-SY5Y (ATCC, CRL-2266) cells were purchased from ATCC. HEK293 and A549 cells were maintained in Dulbecco's modified Eagle's medium (DMEM, Gibco, 11965118)+10% fetal bovine serum (FBS, Gibco, 10082147)+Penicillin-Streptomycin (Gibco, 15140163) in 37° C. incubator with 5% CO2. SH-SY5Y was maintained in 1:1 mixture of Eagle's Minimum Essential Medium and F12 Medium (ATCC, 30-2003)+10% FBS+Penicillin-Streptomycin.

TC1 mES cells (CVCL_M350 were grown under feeder-free ES cell culture conditions. Cells were maintained on 0.2% gelatin (Sigma, G1393-100ML) coated tissue culture plates in mES 2i+LIF medium (DMEM-High Glucose (CORNING, 10-013-CV) supplemented with 15% Fetal Bovine Serum (Gibco, 16140089), 1% Pen Strep (Gibco, 15140-122), 10 mM HEPES (Gibco, 15630-080), 1 mM sodium-pyruvate (Sigma, S8636), 55 M 2-Mercaptoethanol (Gibco, 21985023), 1× GlutaMax (Gibco, 35050061), 1× non-essential amino acids (Gibco, 11140050), 1000 U/mL leukemia inhibitory factor (Sigma-Aldrich, ESG1107), 3 μM CHIR99021 (Stem Cell Technologies, 72054) and 2 μM PD0325901 (Stem Cell Technologies, 72184).

Design of biotinylated antisense oligos. Provided is the strategy for manually designing biotinylated antisense oligos to enrich both human and mouse snoRNAs. The snoRNAs with known target sites serve as positive controls and used to test their potential interactions with other RNA targets. These snoRNAs include SNORD18, SNORD27, SNORD99, SNORA21 and SNORA70 (associated with spliceosome) (Falaleeva et al., PNAS USA 113, E1625-1634), U32a, U33, and U35a (involved in lipid metabolism) (Michel et al., Cell Metab 14, 33-44 (2011)), SNORD60 (Brandis et al., The Journal of biological chemistry 288, 35703-35713, (2013)), SNORD88C (precursor to miRNAs) (Scott et al., Nucleic acids research 40, 3676-3688, (2012)), and U17 (ribosomal RNA processing and cholesterol trafficking) (Jinn et al., Cell Metab 21, 855-867. (2015)). HGNC approved gene symbols are used wherever possible to avoid confusions. Two to three antisense oligos were designed for each family of snoRNAs (3 oligos for each of SNORA53, SNORA73 and SNORD50). We designed the oligos using ChIRP-designer (biosearchtech.com/chirp-designer), and manually adjusted the sequences based on human-mouse sequence alignments. To make oligos that bind both human and mouse snoRNA homologs, we use G against C/U, and T against A/G nucleotides. Many mouse snoRNA genes were not annotated correctly, and were extended manually to match the human homologs. In total the 96 oligos target 46 families of snoRNAs, including 10 with known targets and 36 without known targets. The oligos were synthesized at a scale of 9.99+/−0.22 nmole each.

snoRNAs enrichment and PARIS2 library construction. HEK293 and SH-SY5Y cells cultured to 80% confluency in 10 cm dish. Cells were washed twice with PBS, and then treated with 2.0 mg/ml amotosalen in PBS for 15 minutes in 37° C. incubator. The cells in crosslinking solution were placed on ice trays in Stratalinker 2400 UV crosslinker and irradiated for 30 minutes under 365 nm UV bulbs. The crosslinked RNAs were extracted using TNA method 13. Target snoRNAs were enriched according to the Chromatin Isolation by RNA Purification (ChIRP) Protocol 14. Briefly, 100 g of total RNA was prepared in 500 μL Hybridization buffer (500 mM NaCl, 0.7% SDS, 33 mM Tris-HCl pH 7.0, 0.7 mM EDTA, 10% formamide and 1U SUPERaseIn). 20 μL of 10 μM biotin labeled probe mixtures (2.0 picomole/each probe×116 probes=232 picomoles) were added for hybridization at 37° C. for 6 hours. After hybridization, Dynabeads™ MyOne™ Streptavidin C1 (Invitrogen, 65001) and DynaMag-2 magnet strip (Invitrogen, 12321D) were used to pull down the targeted snoRNAs. After stringency washes, enriched snoRNAs were released by adding 10 μL of DNase I (Invitrogen, AM2222) and purified by ethanol precipitation methods. The PARIS2 sequencing libraries were constructed as previously described in detail Zhang et al., Nat Commun 12, 2344, (2021).

Chromosome-associated RNA (caRNA) extraction. We prepared human iPS cells and iPS-derived cell lines, including Neuro progenitor cells (NPC), endothelial cells (EC), Astrocyte and Neuron cells 16. After UV365 nm crosslinking with 2.0 mg/ml amotosalen, 5×106 to 1×107 cells were collected and washed with 1 mL cold PBS (with 1 mM EDTA buffer), then centrifuged at RT with 500 g to collect the cell pellet. 200 μL of ice-cold lysis buffer (10 mM Tris-HCl, pH=7.5, 0.05% NP40, 150 mM NaCl) was added to the pellet and gently pipetted up and down 3-5 times to resuspend the cells. The lysate was incubated on ice for 5 minutes. Gently pipetted up the cell lysate over 500 μL (2.5 volumes) of chilled sucrose cushion (24% RNase-free sucrose in lysis buffer) and then centrifuged at 4° C. with 3,500 g for 10 minutes. 200 μL of ice-cold PBS (with 1 mM EDTA) was gently added to the nuclei pellet without dislodging the pellet, and the PBS/EDTA was aspirated. The nuclei pellet was resuspended in 200 μL prechilled glycerol buffer (20 mM Tris-HCl, pH=7.9, 75 mM NaCl, 0.5 mM EDTA, 0.85 mM DTT, 0.125 mM PMSF, 50% glycerol) with gentle flicking of the tube. An equal volume of cold nuclei lysis buffer (10 mM HEPES, pH=7.6, 1 mM DTT, 7.5 mM MgCl2, 0.2 mM EDTA, 0.3 M NaCl, 1 M UREA, 1% NP-40) was added and vortexed vigorously for 2-5 seconds. The nuclei pellet mixtures were incubated for 2 minutes on ice, then centrifuged at 4° C. with 15,000 g for 2 minutes. The supernatant was collected as a soluble nuclear fraction (nucleoplasm). The pellet was gently rinsed with cold PBS/1 mM EDTA without dislodging and then collected as a chromosome-associated fraction. For crosslinked samples: a 21-gauge needle and syringe were used to fully solubilize the pellet, standard TNA method was used to purify the chromosome-associated RNA 15. All the libraries in this study were sequenced using NovaSeq 6000 (SE100 bp) by Medgenome Inc.

Optimized denatured RiboMeth-seq (dRMS) for small RNAs. RiboMeth-seq (RMS) uses the resistance of 2′-O-methylated ribose to alkaline hydrolysis to determine methylation level. While this method works well for some RNAs, e.g., 18S and 28S rRNAs, as has been demonstrated in numerous studies, tRNAs and many other small ncRNAs, e.g., snRNAs, pose unique challenges, due to their small size, high modification density and high structure stability. Standard alkaline hydrolysis condition, e.g., 100 mM bicarbonate, pH 9, and 95° C., cannot completely denature ncRNAs, leading to longer fragments and biased cleavage of single stranded regions, e.g., internal loops 17-19. For example, tRNAs have highly stable tertiary structures that persist at temperatures above 95° C. in aqueous buffer 20. tRNAs have a high density of modifications, ˜13 per tRNA, or ˜1 per 6 nts, many of which block reverse transcription and create cDNA ends that are not due to 2′-O-methylation. The longer fragments block detection of Nm sites in short ncRNAs. For example, modification sites near the 5′ or 3′ ends can only be detected using the corresponding ends of the ends, reducing sensitivity. The 5′ and 3′ ends often produce different patterns, making it even harder to accurately quantify the methylation levels. Uneven fragmentation leads to highly variable coverage, and some positions are extremely low. In addition, some of the RT enzymes, e.g., Superscript SSIV, produce untemplated 3′ end additions, e.g., one or two Ts, making the 3′ end detection unreliable.

Total RNA was isolated using TRIzol following the manufacturer's instructions. After running 10% (wt/vol) urea polyacrylamide gel, small RNA (70-300 nt) was excised and purified using ZR small-RNA PAGE Recovery Kit (Zymo Research, R1070). 100 ng of small RNA (70-300 nt) was denatured in 95% DMSO solutions for 3 minutes at 98° C. Then, 10 mM sodium bicarbonate buffer was added to each reaction to perform hydrolysis at different pH (9, 10, and 11) for different times (5, 10, and 15 minutes). Fragmented RNAs were purified using ethanol precipitation and resuspended with 20 μL of RNase-free water. Purified fragmented RNAs were treated with AlkB as described below. AlkB treatment of RNA was performed in incubated in 50 μL reaction mixture containing 50 mM HEPES (pH 8.0), 75 M ferrous ammonium sulfate (pH 5.0), 1 mM α-ketoglutaric acid, 2 mM sodium ascorbate, 50 mg/L bovine serum albumin, 4 g/mL AlkB, 2,000 U/mL RNase inhibitor and 200 ng RNA at 37° C. for 60 minutes. AlkB reaction buffer was prepared fresh prior to each use. The RNA was recovered by ethanol precipitation.

The fragmented RNAs were incubated in 50 μL reaction mixture containing 5 μL 10×PNK buffer (New England Biolabs, B0201S), 1 mM ATP (New England Biolabs, P0756S), 10 U T4PNK (New England Biolabs, M0201L) and 200 ng RNA at 37° C. for 30 minutes. Reactions were stopped by incubation at 75° C. for 5 minutes, followed by ethanol precipitation, and resuspension of the washed pellet in water. An adapter was ligated to end-repaired RNA fragments using high concentration T4 RNA ligase 1 (New England Biolabs, M0437) in 20 μL reaction mixture containing 1×T4 RNA ligase buffer, 5 mM DTT, 12.5% v/v PEG8000, 10% DMSO, and 1U SuperaseIn, the reaction was incubated at room temperature for 3 hours. After adapter ligation, 60 units of RecJf (New England Biolabs, M0264), and 50 units of 5′ deadenylase (New England Biolabs, M0331) were added to remove free adapters. RNA was purified with Zymo RNA clean and Concentrator-5 Kit (Zymo Research, R1016). An RT-step and PCR amplification were coupled with barcoding. The resulting library is sequenced in single-end mode (SE101) using the NovaSeq 6000 sequencer.

RNA knockdown studies. DsiRNAs were designed and purchased from Integrated DNA Technologies (IDT) to target human FBL and DKC1 genes. ASOs were manually designed to target the sequence of conserved regions between human and mouse snoRNAs (SNORD97 and SNORD133). ASOs were synthesized by IDT with phosphorothioate backbones and 2′-O-methoxyethyl modifications (2′-MOE) to increase hybridization affinity and stability. HEK293, A549 and TC1 mES were respectively cultured in a 6-well plate at 500,000 cells/well for 24 hours prior to the experiment. On the day of transfection, cells were transfected with 20 nM of DsiRNAs (targeting FBL and DKC1 in HEK293 and A549 cells) and 50 mM of ASOs (targeting Snord97 and Snord133 in mES) using Lipofectamine RNAiMAX Reagent (Invitrogen™, 13778150) according to the manufacturer's protocol. After 3 days, HEK293 and A549 cells were transfected again with 10 nM of DsiRNAs, and then were collected two more days later. For mES, cells were collected on day 2 and day 5 after transfection to detect the knockdown efficiency by RT-qPCR. Total RNA was extracted using TRIzol reagent (Invitrogen™, 15596018). The sequences of DsiRNAs and ASOs are listed in Example 2.

Total RNAs were isolated using TRIzol reagent after KD. For EB formation assay, mES were replated into ultra-low attachment plates within CMD medium at day 2 after knocking down of specific snoRNAs by ASO. After another 3 days of incubation, EBs were collected for total RNA extraction and RT-qPCR. 1 g of total RNAs were first transcribed into complementary DNA (cDNA) using SuperScript™ III reverse transcriptase (Invitrogen, 18080093) with random hexamers. The cDNA was then used as the template for the real-time PCR reaction (qPCR).

LC-MS (ESI) detection of RNA modifications. tRNAs and 18S/28S rRNAs were enriched from total RNAs by gel extraction. 150 ng of RNA samples was resuspended in 20 L of 25 mM NH₄OAc (pH 7.5). RNA samples were heated for 3 minutes at 100° C. to denature the nucleic acid, and immediately chilled on ice, followed by adding 50 ng 13C Adenosine to each sample. 10 U of Benzonase (Millipore, 70664), 2 U of Alkaline Phosphatase (Sigma, 1071302300), 0.6 U of nuclease P1 (Sigma, N8630), 0.2 U of Phosphodiesterase I (Sigma, P3243), 200 ng of Pentostatin (Sigma, SML0508), and 500 ng of tetrahydrouridine (Sigma, 584222) were added to digest tRNA into nucleosides by overnight incubation at 37° C. Separate stock solutions of cytidine, uridine, and their 2′-O-methylated counterparts were prepared at 10 mg/mL in water, adenosine and 2′-O-methyl-adenosine were prepared at 2 mg/mL in water, and guanosine and 2′-O-methyl-guanosine was prepared at 0.5 mg/mL in water. Calibration working standards were prepared from these stock solutions at 5000, 2000, 1000, 500, 200, 100, 50, and 10 ng/mL concentrations in 25 mM ammonium acetate. In separate LC-MS vials, 100 ng of internal standard (¹³C adenosine) was mixed with 60 μL of each standard to match the concentration of the internal standard in the samples.

Samples and standards were analyzed using a Synergi Fusion (4 m particle size, 80 Å pore size, 250×2 mm; Phenomenex) on an Agilent 1290 Infinity II HPLC connected to a Sciex 6500+QTRAP mass spectrometer equipped with an electrospray ion source (ESI). The elution scheme used, at a flow rate of 0.35 mL/min, began with 100% solvent A (5 mM ammonium acetate) with a gradient to 8% solvent B (LC-Ms grade acetonitrile, J.T. Baker) at 10 minutes, 40% Solvent B at 20 minutes and then back to 100% solvent A for 10 minutes. Samples and standards were analyzed in positive ion mode with ESI parameters of 350° C. gas temperature, 3000 V capillary voltage and gas flow of 20 L/min. The MRM parameters are in Table 1.

TABLE 1
MRM parameters.

| | | | Dwell | Declustering | Collision |
|---|---|---|---|---|---|
| | Q1 | Q2 | time | Potential | Energy |
| Compound | (Da) | (Da) | (msec) | (V) | (V) |
| Cytidine | 244.2 | 112.2 | 50.0 | 68.0 | 17.0 |
| Uridine | 245.3 | 113.3 | 50.0 | 69.0 | 16.0 |
| Adenosine | 268.2 | 136.1 | 50.0 | 101.0 | 23.0 |
| Guanosine | 283.7 | 152.1 | 50.0 | 58.0 | 17.0 |
| 2′-O-methyl-cytidine | 258.0 | 111.9 | 50.0 | 88.0 | 12.0 |
| 2′-O-methyl-uridine | 259.0 | 113.2 | 50.0 | 64.0 | 15.0 |
| 2′-O-methyl-adenosine | 281.9 | 135.6 | 50.0 | 30.0 | 21.0 |
| 2′-O-methyl-guanosine | 298.2 | 152.1 | 50.0 | 117.0 | 19.0 |
| ¹³C adenosine | 273.0 | 136.0 | 50.0 | 50.0 | 22.0 |

Oxidative stress treatment. Sodium (meta) arsenite (Sigma, S7400) was dissolved in distilled water to prepare a stock solution. The wild type and KD cells of HEK293, A549 and mES were cultured in a 10-cm dish for 24 hours prior to the treatment. Each cell line was treated with 250 μM sodium arsenite for 4 hours in a 37° C. incubator with 5% CO2. Cells were collected after treatment and total RNA was extracted using TRIzol reagent. 2 μg total RNAs were analyzed by 12% Urea-PAGE gel.

tRNA in vitro angiogenin (ANG) cleavage assay. tRNAs of HEK293/A549 cells were purified from total RNAs by gel extraction. 20 ng of purified tRNAs were used for ANG cleavage reaction with 1 g recombinant human ANG protein (PROSPEC, pro-1903) in buffer 30 mM HEPES PH 7.4, 30 mM NaCl, 5 mM MgCl₂, 0.01% BSA. The in vitro tRNA cleavage was performed at 37° C. for 0/10/20/40 mins. Cleavage products were purified using phenol and alcohol precipitation and were electrophoresed through a denaturing polyacrylamide gel (12%) and stained with SYBR Gold (Invitrogen, S11494). The results were visualized by iBright FL1000 Imaging Systems (ThermoFisher). The electrophoresis gel quantification of cleavage tRNA was analyzed by iBright Analysis Software (Version 5.1.0). Background was corrected using rolling ball radius (53) subtraction.

Melting curve analysis. For each annealing reaction, 50 ng of purified tRNAs were combined with 2× hybridization buffer (10 mM NaCl, 20 mM Tris-HCl pH 7.5, and 2 mM EDTA), 1 μL of 20×SYBR Green (Invitrogen™, S7563) and made up to a final volume of 20 μL with H₂O. FBL knockdown samples and control were loaded to a 96-well plate in two biological replicates and five technical replicates. Samples were heated at 95° C. for 15 s and then rapidly cooled to 4° C. held for 1 min to facilitate annealing of tRNAs. A single fluorometric data point was collected for every 1° C. increment as the temperature was raised back to 90° C. The normalized reporter (Rn) and the derivative reporter (−Rn′) were exported from Applied Biosystems StepOnePlus Real-Time PCR machine. Rn is calculated as the fluorescence signal from the reporter dye normalized to the fluorescence signal of the passive reference. −Rn′ is calculated as the negative first derivative of the normalized fluorescence (Rn) generated by the reporter during PCR amplification, which was plotted to determine the Tm value.

Cell proliferation assay. CellTiter-Blue® Cell Viability Assay kit (Promega, G8080) was used to measure cell viability for human and mouse cell lines. For each cell line, 2000 cells were added to each of the triplicate wells of a 96-well plate. Cell metabolism rate was measured every day according to the kit manufacturer's instructions. The fluorescence signal was read by Synergy HTX Microplate Multimode Reader with excitation at 560 nm and emission at 590 nm.

Bulk RNA-seq library preparation. Total RNA (1-2 ug) was used as starting materials for ribosomal RNA depletion by using rRNA Depletion Kit v2 (Human/Mouse/Rat) (NEB, E7405L). After beads purification, fragmentation was performed using NEBNext® Magnesium RNA Fragmentation Module (NEB, E6150S) by incubation with fragmentation buffer (10×) at 94° C. for 5 min. The fragmented RNAs were repaired by PNK treatment, and bulk RNA-seq libraries were prepared as previously described 15.

Ribosome profiling (ribo-seq) library preparation. Ribosome profiling was performed as previously described in Ingolia et al., Science 324, 218-223 (2009), and Ingolia et al., Nature protocols 7, 1534-1550 (2012) with minor modifications to the RNase digestion step. Two experimental replicates were harvested for each cell line, with three individual 10-cm dish of cells collected for each replicate. Cells were cultured to 80% confluency and washed with ice-cold PBS. After removing PBS thoroughly, cells were flash frozen in liquid nitrogen. Then 400 μL of ice-cold lysis buffer was added to lyse the cells on wet ice for 10 minutes. Cells were then scraped off into a 1.5 mL tube and NP40 was added to the final concentration of 1%. Lysates were homogenized by pipetting and triturated ten times through a 25-gauge needle if the cell lysates were not clear. The lysate was cleared by centrifugation at 2,000 g 4° C. for 10 minutes, and then 20,000 g 4° C. for 10 minutes. The supernatant was transferred to a fresh microfuge tube. Lysate RNA concentration was measured with a Qubit HS RNA kit and an aliquot amount of lysates was used for digestion with 600 ng RNase A and 75 U RNase T1/g RNA at 25° C. for 30 minutes. Reaction was stopped by chilling on ice and adding 50 U SUPERase In RNase inhibitor (2.5 μL/sample) and 0.5 M EDTA (100×). Following nuclease digestion, monosomes were purified using ultracentrifugation in an SW55i rotor at 55,000 rpm 4° C. for 5 hours. Then RNAs were extracted using the TRIzol method and 15% polyacrylamide TBE-Urea gel was used to isolate ribosome-protected fragments (RPFs) (26-34 nt). Recovered RPFs were quantified and followed by ribosomal RNA depletion using Illumina Ribo-Zero Plus rRNA Depletion Kit (Illumina, 20040526). After PNK treatment, and Ribosome profiling libraries were prepared. The second round of rRNA depletion was performed after the circularization step using pre-synthesized biotinylated probes.

HPG labeling for monitoring global protein synthesis. A click chemistry-based approach was employed to label newly synthesized proteins. In brief, one 10-cm plate of cells was transferred to a methionine-free medium (Thermo Fisher, 21013024) and additional L-Glutamine (2 mM) was added. Cytosolic and mitochondrial ribosomes are allowed to incorporate the alkyne-containing methionine homolog (HPG, Thermo Fisher, C10186) into newly generated proteins after 30 minutes incubation. Then the cells were washed with PBS containing cycloheximide (Chx, 50 g/mL, RPI) and homogenized with buffer containing 50 mM Tris (pH 8.8), 1 mM PMSF and 1% SDS. The total proteins incorporating HPG were clicked to an azide conjugated fluorophore through copper-catalyzed Huisgen cycloaddition (click), The click reaction was conducted on ice for 30 minutes using Click-iT Cell Reaction Buffer Kit (Thermo Fisher, C10269) with 80 M Alexa Fluor 647-azide (Thermo Fisher, A10277). The proteins were then purified from the mixture using a MeOH/chloroform approach after the completion of the click reaction. The resulting pellet was completely dried by heating it to 50° C. and then dissolved in the buffer containing 50 mM Tris (pH 8.8), 1 mM PMSF and 1% SDS at 37° C. Then the samples were loaded onto a 4-12% Bis-Tris gel (Thermo Fisher, NP0322BOX), 180 V for 60 minutes. The fluorescent signals were visualized by iBright FL1000 Imaging Systems. Coomassie blue staining was also performed to visualize the loading amount for normalization. The lane intensities were quantified by iBright Analysis Software (Version 5.1.0).

Detection of mitochondrial-encoded nascent peptides. A click chemistry-based approach was employed to label newly synthesized mitochondrial-encoded peptides which was described in Yousefi et al., EMBO Rep 22, e51635 (2021) with minor modification. In brief, a 10-cm plate of cells was transferred to a methionine-free medium (Thermo Fisher) and additional L-Glutamine (2 mM) was added. To halt cytosolic translation, cells were treated with cycloheximide (Chx, 50 g/mL, RPI) for 20 min. For control experiments aimed at blocking mitochondrial translation, chloramphenicol (Ch1, 150 g/mL) was added 50 min prior to the initiation of labeling. Subsequently, 500 M of L-Homopropargylglycine (HPG, Thermo Fisher) was introduced and allowed to incubate for 4 h. After harvesting the cells, wash the cells with PBS twice. Then cells were dissolved in an isolation buffer (5×, 1.25 mM sucrose, 50 mM KCl, 50 mM HEPES pH 7.4, 10 mg/ml BSA, and 10 mM PMSF). Subsequently, the cells (500 uL in 2 mL tube) were homogenized on ice using a tissue homogenizer with level 1 for 1 s on 1 s off, 5 cycles. Then mitochondria were isolated through differential centrifugation. An initial centrifugation step is at 400×g for 10 min at 4° C., followed by a second centrifugation at 800×g for 7 min at 4° C. to remove the cell debris. Then the mitochondria were collected by centrifugation at 10,000×g for 10 min at 4° C. The freshly isolated mitochondria can be observed at the bottom of the tube, then washed in an isolation buffer without BSA (5×, 1.25 mM sucrose, 50 mM KCl, 50 mM HEPES pH 7.4, and 10 mM PMSF. Pelleted mitochondrial proteins (better to determine the protein concentration) were dissolved in 25 L of 50 mM Tris (pH 8.8) containing 1 mM PMSF and 1% SDS overnight to ensure complete dissolution. Before click reaction, samples were shortly spun down to remove insoluble matter. A click reaction was conducted on RT/ice using a commercial kit (Click-iT Cell Reaction Buffer Kit, Thermo Fisher) with 80 M Alexa Fluor 647-azide (Thermo Fisher) for 30 min. Then the proteins were purified from the mixture using a MeOH/chloroform approach after the completion of the click reaction. The resulting pellet was completely dried by heating it to 50° C. and then dissolved in a loading buffer comprised of 7.5 M Urea, 100 mM Dithiothreitol (DTT), and 1% benzonase at 37° C. for 20 minutes. Then the samples were loaded onto a 10-20% Tris-Tricine gradient gel, 125 V for 120 minutes. Then the results were visualized by iBright FL1000 Imaging Systems (ThermoFisher). The fluorescent signals within the gels were analyzed by iBright Analysis Software (Version 5.1.0).

Luciferase Reporter Assay. pcDNA3 RLUC POLIRES FLUC is a bicistronic reporter construct encoding Cap-dependent Renilla and Poliovirus-IRES-dependent firefly luciferase (Addgene, 45642; RRID: Addgene_45642)) (Poulin et al., J Biol Chem 273, 14002-14007, (1998)). The vector was used to construct the reporter plasmid, which contained both a firefly luciferase (R-luc), and a Renilla luciferase (F-luc). F-luc-6×Met (ATG) reporter plasmid was obtained by inserting the 6×Met before the F-luc coding region. The inserted sequences were added by designing amplification PCR primers and F-luc mutant reporters were cloned by inserting sequences into pcDNA3 RLUC POLIRESFLUC vector backbone replacing the original sequences between BamHI site and BsiWI site. 10⁵ cells including wild type and knockout cells in 24-well culture plates were transiently transfected with 1 μg of each plasmid DNA using Lipofectamine 3000 (Invitrogen, L3000001) according to the manufacturer's instructions. After 24 h, the cells were washed with PBS and resuspended in 75 μL of DMEM, then 75 μL Dual-Glo Luciferase Assay Reagent was added into the plate to allow for cell lysis to occur after at least 10 min incubation. Firefly luminescence was measured using a luminometer. Next, 75 μL Dual-Glo Stop & Glo Reagent was added into the plate. After at least 10 minutes, Renilla luminescence was measured. The renilla luciferase expression in transfected cells allowed us to correct for variation in transfection efficiency. The luciferase activities were measured using the Dual-Luciferase Reporter Assay System (Promega, E1910), according to the manufacturer's instructions by a FLUOstar Omega multi-mode microplate reader. All the luciferase assays were performed at least in triplicates.

Western blot. For the detection of expression levels of selected marker genes in mES cells, WT and snoRNA-KO cell lines were collected and lysed in RIPA buffer (150 mM NaCl, 50 mM Tris, 2 mM MgCl2, 0.1% SDS, 0.4% DOC, 0.04% Triton X-100, 2 mM DTT, and complete protease inhibitors). The following antibodies were used for blotting overnight at 4° C. in TBST+5% dried milk: Sox2 antibody (Cell Signaling, 2748), Oct4 antibody (Abcam, ab19857), Nanog Polyclonal Antibody (Thermo Fisher, A300-397A), beta-Actin antibody (Cell Signaling, 4970).

snoRNA conservation analysis. For the detection of expression levels of selected marker genes in mES cells, WT and snoRNA-KO cell lines were collected and lysed in RIPA buffer (150 mM NaCl, 50 mM Tris, 2 mM MgCl2, 0.1% SDS, 0.4% DOC, 0.04% Triton X-100, 2 mM DTT, and complete protease inhibitors). The following antibodies were used for blotting overnight at 4° C. in TBST+5% dried milk: Sox2 antibody (Cell Signaling, 2748), Oct4 antibody (Abcam, ab19857), Nanog Polyclonal Antibody (Thermo Fisher, A300-397A), beta-Actin antibody (Cell Signaling, 4970). We analyzed the conservation of snoRNAs to assess their potential in base pairing with other RNAs as follows.
1. For each snoRNA, download all the sequences from Rfam (rfam.xfam.org/), for example SNORA18, and select the SNORA18 family (rfam.xfam.org/family/RF00425), then download the sequences here: rfam.xfam.org/family/RF00425 #tabview=tab1 (Download unaligned sequences (fasta)).
2. To analyze the conservation of the snoRNAs, first align the sequences using Clustal Omega and download results in clustal format (ebi.ac.uk/Tools/msa/clustalo/), and then convert the clustal alignments back to fasta format (sequenceconversion.bugaco.com/converter/biology/sequences/clustal_to_fasta.php).
3. To visualize the alignments and examine sequence conservation, edit the alignments in Jalview to remove nucleotide positions that are only present in less than 30% sequences. Take note of the alignment lengths.
4. Manually edited alignments are visualized using the Weblogo server after replacing T with U. Generate the weblogo figure using this web server: weblogo.threeplusone.com/create.cgi. Here are the parameters: Output format: PDF, Sequence type: RNA, Stacks per line: length of the alignment from Jalview (for example, 139 for RF00425). No error bars. No version fineprint. Download the pdf as follows: RFxxxxx_all_manualT2U_weblogo_3+1_139.pdf (as an example).

snoRNA target prediction. PLEXY was used to predict target RNAs for C/D box snoRNAs (Kehr et al., Bioinformatics 27, 279-280, (2011)). RNAsnoop was used to predict target RNAs for H/ACA box snoRNAs with default parameter Tafer et al., Bioinformatics 26, 610-616, (2010)). Predicted targets on tRNAs were aligned to the standard tRNA model for visualization, therefore the positions of some sites are lifted from their original loci and different from them.

PARIS2 data analysis. Non-continuous reads from PARIS2 and PAR-CLIP were extracted using the published CRSSANT pipeline (Zhang et al., Genome Res 32, 968-985, (2022)). Briefly, after mapping reads using the optimized STAR parameters, duplex groups (DGs) were assembled, color-coded, and visualized in IGV (Thorvaldsdottir et al., Briefings in bioinformatics 14, 178-192, (2013)). Manually curated genomes with snoRNAs and targets assembled on the same “chromosome” were used to enable visualization of intermolecular interactions on the same track. For each DG, the coverage fraction was calculated as described before, covfrac=c/sqrt(a*b), where c is the number of chimeric reads, a and b are the numbers of coverage at the two sides of the interactions.

RiboMethSeq data analysis. RMS and dRMS sequencing data were preprocessed to remove adapters from the 3′end using Trimmomatic (v0.36) (Bolger et al., Bioinformatics 30, 2114-2120, (2024)). PCR duplicates were removed using the readCollapse script from the icSHAPE pipeline. After primary preprocessing, reads were mapped to the hg38 genome using the STAR program (2.7.9a) (Dobin et al., Bioinformatics 29, 15-21, (2013)). The parameters used are as follows: STAR --runMode alignReads --runThreadN 8 --genomeDir staridx_2.7.9 --genomeLoad NoSharedMemory --readFilesln Sample.fastq --outFileNamePrefix Outprefix --outStd Log --outReadsUnmapped Fastx --outSAMtype BAM Unsorted SortedByCoordinate --outSAMmode Full --outSAMattributes All --outFilterType BySJout --outFilterMultimapNmax 80 --outFilterMultimapScoreRange 1 --outFilterScoreMin 1 --outFilterScoreMinOverLread 0.1 --outFilterMatchNmin 15 --outFilterMatchNminOverLread 0.1 --outFilterMismatchNmax 5 --outFilterMismatchNoverLmax 0.1 --outFilterMismatchNoverReadLmax 0.1 --alignIntronMin 20 --alignIntronMax 1000000. The number of 5′ and 3′ reads were calculated with bedtools genomecov (v2.29.2). tRNA molecules contain abundant modifications that will stop reverse transcription and generate heavy RT-stops. To avoid the false positive results caused by other modifications, the ribosome methylation score (RiboMethScore, or RMscore for short) was computed with four neighboring nucleotides (±2 nucleotide window) from 5′-end and 3′-end reads at nucleotide resolution. The empirical rule with 1.5 standard deviation (μ±1.5*σ, the probability of 1.5 standard deviations is about 86.6%) was also used to filter the discrete values. If there are observations that fall outside the 1.5 standard deviation, only two neighboring nucleotides (±1 nucleotide window) from 5′-end and 3′-end reads were used to calculate RiboMethScore. Statistical significance was determined by Student's t test (P<0.05).

RNA structure and conservation analysis. LocARNA was used to perform structured alignments of homologous sequences using default parameters: global and standard mode (Will et al., PLoS Comput Biol 3, e65, (2007)). R-scape and CaCoFold were used to identify and test covariation among homologous sequences, using the “Predict new structure” function Rivas et al., PLoS Comput Biol 16, e1008387, (2020)). In the CaCoFold algorithm, spurious covariations due to phylogeny are removed. For the analysis of consensus from alignments, positions covered by Human PAR-CLIP and eCLIP data analysis Human PAR-CLIP sequencing data and eCLIP sequencing data were used to analyze the binding profiles of box C/D snoRNPs and box H/ACA snoRNPs on tRNAs. The Gene Expression Omnibus (GEO) accession number of PAR-CLIP were: NOP56 (GSM1067863), NOP58 rep1 (GSM1067861), NOP58 rep2 (GSM1067862), FBL rep1 (GSM1067864), FBL rep2 (GSM2042733), FBL MNase (GSM1067865), DKC1 (GSM1067866), AGO2 (GSM1067869) and sRNA (GSM1067868). The GEO accession number of eCLIP data were: DKC1 rep1 (GSM2423283), DKC1 rep2 (GSM2423284) and Control (GSM2423145). The PAR-CLIP and eCLIP sequencing reads were mapped to hg38 genome using following parameters: STAR --runThreadN 8 --runMode alignReads --genomeDir/project/zhipengl_72/minjiez/database/hg38genmaskaddAllSnord1156/staridx --readFilesln FASTQ --genomeLoad NoSharedMemory --outFilterMultimapNmax 50 --outFilterMismatchNmax 5 --outFilterMismatchNoverLmax 0.1 --outFilterMismatchNoverReadLmax 0.1 --outFilterMultimapScoreRange 1 --alignIntronMin 20 --alignIntronMax 1000000 --outReadsUnmapped Fastx --outSAMattributes All --outSAMtype BAM Unsorted SortedByCoordinate --outFilterType BySJout --outFilterScoreMin 1 --outStd Log --alignEndsType Local --scoreGap -8 --outSAMmode Full.

The enrichment level of each RNA was normalized to the median level of 21 mt-tRNAs (mt-tRNAs), which are not the targets of box C/D snoRNPs and box H/ACA snoRNPs. After normalization, the enrichment ratio of each RNA was calculated to Input sample individually. Only RNA loci with more than 10 reads in each sample were analyzed. Top 200 miRNAs in PAR-CLIP Input sample and Top50 miRNAs in eCLIP Input samples were also served as negative control. An empirical false positive (FP) cutoff of 5% miRNAs means that 5% miRNAs were considered enriched, even though none of them have been shown to bind snoRNP proteins. Any RNAs with enriched ratio higher than the ratios at this cutoff were considered significant. mRNAs and lncRNAs are excluded from the PAR-CLIP analysis because the PAR-CLIP normalization was performed against a small RNA-seq dataset (20-200nts). The following differences in experimental conditions are noted: MNase was used for 1 FBL PAR-CLIP, RNase T1 for all other PAR-CLIP, and RNase I for eCLIP. Libraries using MNase and RNase I have higher overall enrichment than the RNase T1 samples. The differences between FBL RNase T1 (reps 1-2) and MNase data were due to known RNase biases in library preparation (Kishore et al., Genome Biol 14, R45. (2013).

Yeast CLIP data analysis. Yeast snoRNPs CLIP sequencing data 38 was mapped to the yeast genome (S. cerevisiae strain BY4741, available from yeastgenome.org) using STAR programming. After mapping, the read number per million of each RNA was calculated. The reads ratio mapped to snoRNAs in NOP1 (FBL in human), NOP56, and NOP58 CLIP samples were 81.87%, 76.20%, and 68.58%, respectively. To study the snoRNPs binding profiles on other RNAs, all remaining reads after subtracting snoRNAs alignments were used to calculate RPM of target RNAs. Enrichment ratio was determined by the fold change of RPM to control sample. Only RNAs with more than 1 RPM in control sample were analyzed. RNAs with enrichment level higher than 2 (compared to control sample) and were called significant.

Bulk RNA-seq data analysis. The adapter sequences of the sequencing data were preprocessed as described above descriptions in the Ribosome profiling data analysis section. Processed reads were mapped to the hg38 or mm10 genome using STAR program (v2.7.9) (Dobin et al., Bioinformatics (Oxford, England) 29, 15-21, (1013)). RPM (reads per million mapped reads) was used as a normalized gene expression unit to compare the same gene expression between different samples. The primary mapped location on a tRNA gene was counted for all multi-mapped reads.

Ribo-seq data analysis. Sequencing data were preprocessed to remove adapters from the 5′ end and 3′ end using Trimmomatic software (v0.36). PCR duplicates are removed using readCollapse script from the icSHAPE pipeline (Spitale et al., Nature 519, 486-490, (2015)). Reads derived from RPF were aligned to known human rRNA sequences with bowtie2 (v2.2.9). Mapped reads were discarded. Cleaned reads were mapped to the hg38 genome using STAR program (v2.7.9). The parameters used are as follows: STAR --runMode alignReads --runThreadN 8 --genomeDir staridx_2.7.9 --genomeLoad NoSharedMemory --readFilesln Sample.fastq --outFileNamePrefix Outprefix --outStd Log --outReadsUnmapped Fastx --outSAMtype BAM Unsorted SortedByCoordinate --outSAMmode Full --outSAMattributes All --outFilterType BySJout --outFilterMultimapNmax 10 --outFilterMultimapScoreRange 1 --outFilterScoreMin 1 --outFilterScoreMinOverLread 0.1 --outFilterMatchNmin 15 --outFilterMatchNminOverLread 0.1 --outFilterMismatchNmax 5 --outFilterMismatchNoverLmax 0.1 --outFilterMismatchNoverReadLmax 0.1 --alignIntronMin 20 --alignIntronMax 1000000. P-site offsets of ribosome profiling data were determined using the psite script from plastid package (Dunn et al., Bmc Genomics 17. (2016) ARTN 95810.1186/s12864-016-3278-x.). The parameters used are as follows: psite merlin_orfs_rois.txt outputprefix --min_length 25 --max_length 35 --require_upstream --count_files input.bam --min_counts 100 --figformat pdf --dpi 300. Code usage analysis was performed by homemade scripts that are available on GitHub partly at github.com/minjiezhang-usc/snoRNAs_discovery. For nuclear-encoded mRNAs in RNA-seq and ribo-seq from WT and D97 and D133 single KO HEK293 cells, ratios of expression levels were calculated for the top 10% (most up-regulated) and bottom 10% (most down-regulated) mRNAs and weighted by expression levels.

Codon usage analysis. Gene-specific codon usage (GSCU) score was calculated as previously described using the following annotations (Begley et al., Mol Cell 28, 860-870, (2007)). Given that mammalian protein coding genes produce multiple alternative transcripts, the principal transcripts were selected from the APPRIS database for hg38, which contains 36144 records, 20345 genes, 18459 MANE_Select, 23281 PRINCIPAL:1. In total, 15423 principal mRNAs were analyzed for GSCU. The CDS from Ensembl, Homo_sapiens.GRCh38.cds.all.fa, was used as the reference (n=120712 records). Codon usage was performed on the 64 standard codons plus the initiator methionine AUG (m) and recoded UGA for selenocysteine (U or Sec). Amino acid usage was performed on 20 standard amino acids and initiator methionine (m), stop codon (*) and selenocysteine (U or Sec).

Gene set enrichment analysis. GSEA (version 4.3.2) was performed on the gene expression and codon usage differences, using the default parameters for GSEAPreranked: 1000 permutations and No-Collapse (Subramanian et al., Proc Natl Acad Sci USA 102, 15545-15550 (2005)).

The following MSigDB collections were used for human and mouse data: c5.all.v2023.1.Hs.symbols.gmt, m5.all.v2023.1.Mm.symbols.gmt. To summarize the long list of enriched gene ontology (GO) terms, those with positive and negative enrichment nominal p<0.05 are collected and their descriptions clustered based on term similarity, using simplifyEnrichment (Gu et al., Genomics Proteomics Bioinformatics. (2022) 10.1016/j.gpb.2022.04.008.). The following parameters are used: set.seed(0), mat=GO_similarity(goids, ont=“BP”). The restriction of GO terms to biological process (BP) was set to avoid redundancy among BP, molecular function (MF) and cellular component (CC) categories.

### Example 2. Sequences

SNORD97
NC_000012.12:50456571-50456786 Homo sapiens
chromosome 12, GRCh38.p14 Primary
Assembly:
(SEQ ID NO: 1)
ttgcccgatgattataaaaagacgcgttattaagaggactttatg
ctggagttcttgacgtttttctctcttttctatacttctttttct
ttctttgaatgtccagcgtcctgtgagcgaagattatgagatatg
agggcaa.
SNORD97
mRNA transcript (URS00003B57B1_9606)
(SEQ ID NO: 2)
acuucuuuuuuuucuuugaauguccagcuccugugagcgaagauu
augagauaugagggcaa
SNORD133
NCBI Reference Sequence: NC_000011.10
NC_000011.10:c10801608-10801467 Homo sapiens
chromosome 11, GRCh38.p14 Primary
Assembly:
(SEQ ID NO: 3)
cagcccagtgatgatcactattcctacttagagagaataggacta
actttcagaaatccaggcatttttctacctttcatactatctttc
tttcactttacttctcttttctgtcttttatcacttctttctttc
ttcattctttctctcttttgcctggatcgagattgttaagtccct
ctcagtgaagggtaagattatgagatctgagggctg.
SNORD133
mRNA transcript (URS00008E3A5F_9606)
(SEQ ID NO: 4)
cagcccagugaugaucacuauuccuacuuagagagaauaggacua
acuuucagaaauccaggcauuuuucuaccuuucauggaucgagau
uguuaagucccucucagugaaggguaagauuaugagaucugaggg
cug

| DsiRNA name | Duplex sequence |
|---|---|
| hs.DsiRNA Ctrl | 5′-GGUCAAAACUCCCGUGCUGAUCAGU-3′ (SEQ ID NO: 5) |
| | \|\|\|\|\|\|\|\|\|\|\|\|\|\|\|\|\|\|\|\|\|\|\|\|\| |
| | 3′-GUCCAGUUUUGAGGGCACGACUAGUCA-5′ (SEQ ID NO: 6) |
| hs.DsiRNA FBL(1) | 5′-AUGACAAAAUUGAGUACCGAGCCUG-3′ (SEQ ID NO: 7) |
| | \|\|\|\|\|\|\|\|\|\|\|\|\|\|\|\|\|\|\|\|\|\|\|\|\| |
| | 3′-UCUACUGUUUUAACUCAUGGCUCGGAC-5′ (SEQ ID NO: 8) |
| hs.DsiRNA FBL(2) | 5′-AACAUCAUUCCUGUGAUCGAGGAUG-3′ (SEQ ID NO: 9) |
| | \|\|\|\|\|\|\|\|\|\|\|\|\|\|\|\|\|\|\|\|\|\|\|\|\| |
| | 3′-GGUUGUAGUAAGGACACUAGCUCCUAC-5′ (SEQ ID NO: 10) |
| hs.DsiRNA DKC1(1) | 5′-GAAUUUCUUAUCAAACCUGAAUCCA-3′ (SEQ ID NO: 11) |
| | \|\|\|\|\|\|\|\|\|\|\|\|\|\|\|\|\|\|\|\|\|\|\|\|\| |
| | 3′-UUCUUAAAGAAUAGUUUGGACUUAGGU-5′ (SEQ ID NO: 12) |
| hs.DsiRNA DKC1(2) | 5′-ACUAAAGCCUUAUUGAGAAAACAUG-3′ (SEQ ID NO: 13) |
| | \|\|\|\|\|\|\|\|\|\|\|\|\|\|\|\|\|\|\|\|\|\|\|\|\| |
| | 3′-UUUGAUUUCGGAAUAACUCUUUUGUAC-5′ (SEQ ID NO: 14) |

CRISPR-Cas9 sgRNAs
hsSNORD97-sgRNA1
(SEQ ID NO: 15)
taaaaagacgcgttattaagagg
hsSNORD97-sgRNA2
(SEQ ID NO: 16)
gcgaagattatgagatatgaggg
hsSNORD133-sgRNA1
(SEQ ID NO: 17)
ataattagaagaatcgaatctgg
hsSNORD133-sgRNA2
(SEQ ID NO: 18)
ggtaagattatgagatctgaggg
mmSnord133-sgRNA1
(SEQ ID NO: 19)
gttagtcctatcttctctagtgg
mmSnord133-sgRNA2
(SEQ ID NO: 20)
gtggggttctaaccatcaccagg

All publications, patents, and patent documents cited herein are incorporated by reference as though individually incorporated by reference, and in particular, Zhang et al., Proc Natd Acad Sci USA. 2023 Oct. 10; 120(41):e2312126120. doi: 10.1073/pnas.2312126120. Epub 2023 Oct. 4, including all Supplemental data such as datasets 1-6 in .xlsx and .txt format. No limitations inconsistent with this disclosure are to be understood therefrom. The invention has been described with reference to various specific and preferred embodiments and techniques. However, many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

What is claimed is:

1. A method for promoting stem cell differentiation into a mesoderm cell lineage or endoderm cell lineage comprising contacting a stem cell with an agent that reduces or eliminates expression and/or activity of one or more endogenous small nucleolar ribonucleic acid (snoRNA) molecules, wherein the snoRNA molecules comprise SNORD97 and/or SNORD133.

2. The method of claim 1, wherein the SNORD97 comprises a nucleotide sequence according to SEQ ID NO: 1 or SEQ ID NO: 2; and
the SNORD133 comprises a nucleotide sequence according to SEQ ID NO: 3 or SEQ ID NO: 4.

3. The method of claim 1, wherein the agent is one or more of an aptamer, a short interfering RNA (siRNA), a micro-RNA (miRNA), a short hairpin RNA (shRNA), a CRISPR/Cas9 system, an antisense polynucleotide, and a chemical compound.

4. The method of claim 3, wherein the agent comprises the antisense polynucleotide, wherein the antisense polynucleotide specifically binds to at least a portion of any one of SEQ ID NO: 1 to SEQ ID NO: 4.

5. The method of claim 3, wherein the agent comprises one or more of a short interfering RNA (siRNA), a micro-RNA (miRNA), and a short hairpin RNA (shRNA) that specifically binds to at least a portion of any one of SEQ ID NO: 1 to SEQ ID NO: 4.

6. The method of claim 3, wherein the agent comprises a CRISPR/Cas9 system that specifically targets at least a portion of SEQ ID NO: 1 and/or SEQ ID NO: 2.

7. The method of claim 1, wherein the mesoderm cell lineage comprises a cardiac cell selected from the group consisting of a cardiomyocyte, nodal cardiomyocyte, conducting cardiomyocyte, working cardiomyocyte, cardiomyocyte precursor, cardiomyocyte progenitor cell, cardiac stem cell, and cardiac muscle cell.

8. The method of claim 7, wherein the cardiac cell comprises a cardiomyocyte.

9. The method of claim 1, wherein the stem cell comprises a pluripotent embryonic stem cell or an induced pluripotent stem cell.

10. An isolated cardiac cell differentiated from a stem cell, wherein endogenous SNORD97 and/or SNORD133 gene activity of the isolated cardiac cell is i) eliminated or ii) reduced compared to a reference cell.

11. The isolated cardiac cell of claim 8, wherein the isolated cardiac cell is selected from the group consisting of a cardiomyocyte, nodal cardiomyocyte, conducting cardiomyocyte, working cardiomyocyte, cardiomyocyte precursor, cardiomyocyte progenitor cell, cardiac stem cell, and cardiac muscle cell.

12. A composition comprising in vitro differentiated stem cells, wherein the in vitro differentiated stem cells are genetically modified to eliminate or have decreased expression and/or activity of one or more small nucleolar ribonucleic acid (snoRNA) molecules, wherein the snoRNA comprises SNORD97 and SNORD133.

13. The composition of claim 12, wherein the in vitro differentiated stem cells comprise cardiomyocytes.

14. The composition of claim 13, wherein the in vitro differentiated stem cells are derived from human embryonic stem cells or human induced pluripotent stem cells (iPSCs).

15. A method of treating a cardiac disease comprising administering to a subject in need thereof an effective amount of the composition of claim 12, thereby treating the cardiac disease.

16. The method of claim 15, wherein the cardiac disease comprises pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.


SEQUENCE LISTING (31 sequences)

SEQ ID NO: 1 - DNA, 142 bp, Homo sapiens
  source 1..142: mol_type = other DNA
ttgcccgatgattataaaaagacgcgttattaagaggactttatgctggagttcttgacgtttttctctcttttctatacttctttttctttctttgaatgtccagcgtcctgtgagcgaagattatgagatatgagggcaa

SEQ ID NO: 2 - RNA, 142 nt, Homo sapiens
  source 1..142: mol_type = transcribed RNA
  modified_base 1: mod_base = OTHER; note = Uracil
  modified_base 2: mod_base = OTHER; note = Uracil
  modified_base 9: mod_base = OTHER; note = Uracil
  modified_base 12: mod_base = OTHER; note = Uracil
  modified_base 13: mod_base = OTHER; note = Uracil
  modified_base 15: mod_base = OTHER; note = Uracil
  modified_base 27: mod_base = OTHER; note = Uracil
  modified_base 28: mod_base = OTHER; note = Uracil
  modified_base 30: mod_base = OTHER; note = Uracil
  modified_base 31: mod_base = OTHER; note = Uracil
  modified_base 40..42: mod_base = OTHER; note = Uracil
  modified_base 44: mod_base = OTHER; note = Uracil
  modified_base 47: mod_base = OTHER; note = Uracil
  modified_base 52..53: mod_base = OTHER; note = Uracil
  modified_base 55..56: mod_base = OTHER; note = Uracil
  modified_base 61..65: mod_base = OTHER; note = Uracil
  modified_base 67: mod_base = OTHER; note = Uracil
  modified_base 69: mod_base = OTHER; note = Uracil
  modified_base 71..74: mod_base = OTHER; note = Uracil
  modified_base 76: mod_base = OTHER; note = Uracil
  modified_base 78: mod_base = OTHER; note = Uracil
  modified_base 81..82: mod_base = OTHER; note = Uracil
  modified_base 84..88: mod_base = OTHER; note = Uracil
  modified_base 90..92: mod_base = OTHER; note = Uracil
  modified_base 94..96: mod_base = OTHER; note = Uracil
  modified_base 100: mod_base = OTHER; note = Uracil
  modified_base 102: mod_base = OTHER; note = Uracil
  modified_base 109: mod_base = OTHER; note = Uracil
  modified_base 112: mod_base = OTHER; note = Uracil
  modified_base 114: mod_base = OTHER; note = Uracil
  modified_base 124..125: mod_base = OTHER; note = Uracil
  modified_base 127: mod_base = OTHER; note = Uracil
  modified_base 132: mod_base = OTHER; note = Uracil
  modified_base 134: mod_base = OTHER; note = Uracil
ttgcccgatgattataaaaagacgcgttattaagaggactttatgctggagttcttgacgtttttctctcttttctatacttctttttctttctttgaatgtccagcgtcctgtgagcgaagattatgagatatgagggcaa

SEQ ID NO: 3 - DNA, 216 bp, Homo sapiens
  source 1..216: mol_type = other DNA
cagcccagtgatgatcactattcctacttagagagaataggactaactttcagaaatccaggcatttttctacctttcatactatctttctttcactttacttctcttttctgtcttttatcacttctttctttcttcattctttctctcttttgcctggatcgagattgttaagtccctctcagtgaagggtaagattatgagatctgagggctg

SEQ ID NO: 4 - RNA, 216 nt, Homo sapiens
  source 1..216: mol_type = transcribed RNA
  modified_base 9: mod_base = OTHER; note = Uracil
  modified_base 12: mod_base = OTHER; note = Uracil
  modified_base 15: mod_base = OTHER; note = Uracil
  modified_base 19: mod_base = OTHER; note = Uracil
  modified_base 21..22: mod_base = OTHER; note = Uracil
  modified_base 25: mod_base = OTHER; note = Uracil
  modified_base 28..29: mod_base = OTHER; note = Uracil
  modified_base 38: mod_base = OTHER; note = Uracil
  modified_base 44: mod_base = OTHER; note = Uracil
  modified_base 48..50: mod_base = OTHER; note = Uracil
  modified_base 57: mod_base = OTHER; note = Uracil
  modified_base 65..69: mod_base = OTHER; note = Uracil
  modified_base 71: mod_base = OTHER; note = Uracil
  modified_base 75..77: mod_base = OTHER; note = Uracil
  modified_base 80: mod_base = OTHER; note = Uracil
  modified_base 83: mod_base = OTHER; note = Uracil
  modified_base 85: mod_base = OTHER; note = Uracil
  modified_base 87..89: mod_base = OTHER; note = Uracil
  modified_base 91..93: mod_base = OTHER; note = Uracil
  modified_base 97..99: mod_base = OTHER; note = Uracil
  modified_base 102..103: mod_base = OTHER; note = Uracil
  modified_base 105: mod_base = OTHER; note = Uracil
  modified_base 107..110: mod_base = OTHER; note = Uracil
  modified_base 112: mod_base = OTHER; note = Uracil
  modified_base 114: mod_base = OTHER; note = Uracil
  modified_base 116..119: mod_base = OTHER; note = Uracil
  modified_base 121: mod_base = OTHER; note = Uracil
  modified_base 125..126: mod_base = OTHER; note = Uracil
  modified_base 128..130: mod_base = OTHER; note = Uracil
  modified_base 132..134: mod_base = OTHER; note = Uracil
  modified_base 136..137: mod_base = OTHER; note = Uracil
  modified_base 140..141: mod_base = OTHER; note = Uracil
  modified_base 143..145: mod_base = OTHER; note = Uracil
  modified_base 147: mod_base = OTHER; note = Uracil
  modified_base 149: mod_base = OTHER; note = Uracil
  modified_base 151..154: mod_base = OTHER; note = Uracil
  modified_base 158: mod_base = OTHER; note = Uracil
  modified_base 162: mod_base = OTHER; note = Uracil
  modified_base 168..169: mod_base = OTHER; note = Uracil
  modified_base 171..172: mod_base = OTHER; note = Uracil
  modified_base 176: mod_base = OTHER; note = Uracil
  modified_base 180: mod_base = OTHER; note = Uracil
  modified_base 181: mod_base = OTHER; note = Uracil
  modified_base 186: mod_base = OTHER; note = Uracil
  modified_base 193: mod_base = OTHER; note = Uracil
  modified_base 198..199: mod_base = OTHER; note = Uracil
  modified_base 201: mod_base = OTHER; note = Uracil
  modified_base 206: mod_base = OTHER; note = Uracil
  modified_base 208: mod_base = OTHER; note = Uracil
  modified_base 215: mod_base = OTHER; note = Uracil
cagcccagtgatgatcactattcctacttagagagaataggactaactttcagaaatccaggcatttttctacctttcatactatctttctttcactttacttctcttttctgtcttttatcacttctttctttcttcattctttctctcttttgcctggatcgagattgttaagtccctctcagtgaagggtaagattatgagatctgagggctg

SEQ ID NO: 5 - RNA, 25 nt, Synthetic construct
  source 1..25: mol_type = other RNA
  modified_base 3: mod_base = OTHER; note = Uracil
  modified_base 10: mod_base = OTHER; note = Uracil
  modified_base 15: mod_base = OTHER; note = Uracil
  modified_base 18: mod_base = OTHER; note = Uracil
  modified_base 21: mod_base = OTHER; note = Uracil
  modified_base 25: mod_base = OTHER; note = Uracil
ggtcaaaactcccgtgctgatcagt

SEQ ID NO: 6 - RNA, 27 nt, Synthetic construct
  source 1..27: mol_type = other RNA
  modified_base 3: mod_base = OTHER; note = Uracil
  modified_base 6: mod_base = OTHER; note = Uracil
  modified_base 18..21: mod_base = OTHER; note = Uracil
  modified_base 26: mod_base = OTHER; note = Uracil
actgatcagcacgggagttttgacctg

SEQ ID NO: 7 - RNA, 24 nt, Synthetic construct
  source 1..24: mol_type = other RNA
  modified_base 2: mod_base = OTHER; note = Uracil
  modified_base 10: mod_base = OTHER; note = Uracil
  modified_base 14: mod_base = OTHER; note = Uracil
  modified_base 23: mod_base = OTHER; note = Uracil
atgacaaaatgagtaccgagcctg

SEQ ID NO: 8 - RNA, 27 nt, Synthetic construct
  source 1..27: mol_type = other RNA
  modified_base 6: mod_base = OTHER; note = Uracil
  modified_base 10: mod_base = OTHER; note = Uracil
  modified_base 13: mod_base = OTHER; note = Uracil
  modified_base 17..20: mod_base = OTHER; note = Uracil
  modified_base 22: mod_base = OTHER; note = Uracil
  modified_base 25: mod_base = OTHER; note = Uracil
  modified_base 27: mod_base = OTHER; note = Uracil
caggctcggtactcaattttgtcatct

SEQ ID NO: 9 - RNA, 25 nt, Synthetic construct
  source 1..25: mol_type = other RNA
  modified_base 5: mod_base = OTHER; note = Uracil
  modified_base 8..9: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 24: mod_base = OTHER; note = Uracil
aacatcattcctgtgatcgaggatg

SEQ ID NO: 10 - RNA, 26 nt, Synthetic construct
  source 1..26: mol_type = other RNA
  modified_base 3: mod_base = OTHER; note = Uracil
  modified_base 6: mod_base = OTHER; note = Uracil
  modified_base 10: mod_base = OTHER; note = Uracil
  modified_base 19: mod_base = OTHER; note = Uracil
  modified_base 23..24: mod_base = OTHER; note = Uracil
catcctcgatcacaggaatgagttgg

SEQ ID NO: 11 - RNA, 25 nt, Synthetic construct
  source 1..25: mol_type = other RNA
  modified_base 4..6: mod_base = OTHER; note = Uracil
  modified_base 8..9: mod_base = OTHER; note = Uracil
  modified_base 11: mod_base = OTHER; note = Uracil
  modified_base 18: mod_base = OTHER; note = Uracil
  modified_base 22: mod_base = OTHER; note = Uracil
gaatttcttatcaaacctgaatcca

SEQ ID NO: 12 - RNA, 27 nt, Synthetic construct
  source 1..27: mol_type = other RNA
  modified_base 1: mod_base = OTHER; note = Uracil
  modified_base 5..6: mod_base = OTHER; note = Uracil
  modified_base 11..13: mod_base = OTHER; note = Uracil
  modified_base 16: mod_base = OTHER; note = Uracil
  modified_base 23..24: mod_base = OTHER; note = Uracil
  modified_base 26..27: mod_base = OTHER; note = Uracil
tggattcaggtttgataagaaattctt

SEQ ID NO: 13 - RNA, 25 nt, Synthetic construct
  source 1..25: mol_type = other RNA
  modified_base 3: mod_base = OTHER; note = Uracil
  modified_base 10..11: mod_base = OTHER; note = Uracil
  modified_base 13..14: mod_base = OTHER; note = Uracil
  modified_base 24: mod_base = OTHER; note = Uracil
actaaagccttattgagaaaacatg

SEQ ID NO: 14 - RNA, 27 nt, Synthetic construct
  source 1..27: mol_type = other RNA
  modified_base 3: mod_base = OTHER; note = Uracil
  modified_base 5..8: mod_base = OTHER; note = Uracil
  modified_base 10: mod_base = OTHER; note = Uracil
  modified_base 14: mod_base = OTHER; note = Uracil
  modified_base 20..22: mod_base = OTHER; note = Uracil
  modified_base 25..27: mod_base = OTHER; note = Uracil
catgttttctcaataaggctttagttt

SEQ ID NO: 15 - DNA, 23 bp, Synthetic construct
  source 1..23: mol_type = other DNA
taaaaagacgcgttattaagagg

SEQ ID NO: 16 - DNA, 23 bp, Synthetic construct
  source 1..23: mol_type = other DNA
gcgaagattatgagatatgaggg

SEQ ID NO: 17 - DNA, 23 bp, Synthetic construct
  source 1..23: mol_type = other DNA
ataattagaagaatcgaatctgg

SEQ ID NO: 18 - DNA, 23 bp, Synthetic construct
  source 1..23: mol_type = other DNA
ggtaagattatgagatctgaggg

SEQ ID NO: 19 - DNA, 23 bp, Synthetic construct
  source 1..23: mol_type = other DNA
gttagtcctatcttctctagtgg

SEQ ID NO: 20 - DNA, 23 bp, Synthetic construct
  source 1..23: mol_type = other DNA
gtggggttctaaccatcaccagg

SEQ ID NO: 21 - DNA, 14 bp, Synthetic construct
  source 1..14: mol_type = other DNA
atgtccagcgtcct

SEQ ID NO: 22 - DNA, 29 bp, Synthetic construct
  source 1..29: mol_type = other DNA
tgagcgaagattatgagatatgagggcaa

SEQ ID NO: 23 - DNA, 15 bp, Synthetic construct
  source 1..15: mol_type = other DNA
ataggactaactttc

SEQ ID NO: 24 - DNA, 12 bp, Synthetic construct
  source 1..12: mol_type = other DNA
attgttaagtcc

SEQ ID NO: 25 - DNA, 20 bp, Synthetic construct
  source 1..20: mol_type = other DNA
ggtaagattatgagatctga

SEQ ID NO: 26 - DNA, 23 bp, Synthetic construct
  source 1..23: mol_type = other DNA
taaaaagacgcgttattaagagg

SEQ ID NO: 27 - DNA, 23 bp, Synthetic construct
  source 1..23: mol_type = other DNA
gggagtatagagtattagaagcg

SEQ ID NO: 28 - DNA, 23 bp, Synthetic construct
  source 1..23: mol_type = other DNA
ataattagaagaatcgaatctgg

SEQ ID NO: 29 - DNA, 23 bp, Synthetic construct
  source 1..23: mol_type = other DNA
gggagtctagagtattagaatgg

SEQ ID NO: 30 - DNA, 20 bp, Synthetic construct
  source 1..20: mol_type = other DNA
catatctcataatcttcgct

SEQ ID NO: 31 - DNA, 20 bp, Synthetic construct
  source 1..20: mol_type = other DNA
tcagatctcataatcttacc